SEQUENCE LISTING (9 sequences)

SEQ ID NO: 1 - DNA, 31 bp, Artificial Sequence
  oligonucleotide primers
gggggatccatgtcggtggttgggctagacg

SEQ ID NO: 2 - DNA, 24 bp, Artificial Sequence
  oligonucleotide primers
gcaagcagttcaagcccacaatct

SEQ ID NO: 3 - DNA, 30 bp, Artificial Sequence
  oligonucleotide primers
acgcgtcgacgatccttcttaaaaatctac

SEQ ID NO: 4 - DNA, 28 bp, Artificial Sequence
  oligonucleotide primers
gtaaagcttggctggcccggtccgcctc

SEQ ID NO: 5 - DNA, 25 bp, Artificial Sequence
  oligonucleotide primers
gggggaattcctaggatgggcaaag

SEQ ID NO: 6 - DNA, 26 bp, Artificial Sequence
  oligonucleotide primers
ggcggaattcgatgccctttcaagaa

SEQ ID NO: 7 - DNA, 20 bp, Artificial Sequence
  oligonucleotide primers
acataaggctgagcgattgg

SEQ ID NO: 8 - DNA, 23 bp, Artificial Sequence
  oligonucleotide primers
atgtagcagctctgtgagcctac

SEQ ID NO: 9 - DNA, 19 bp, Artificial Sequence
  oligonucleotide primers
caggaagatcgcactccag

(19) United States
(12) Patent Application Publication
(10) Pub. No.: US 2011/0047631 A1
(43) Pub. Date: Feb. 24, 2011

(54) HEAT SHOCK PROTEIN DEFICIENCIES AS MODEL SYSTEMS FOR BRAIN PATHOLOGY AND CANCER

(75) Inventors: Nahid F. Mivechi, Martinez, GA (US); Binnur Eroglu, Evans, GA (US); Dimitrios Moskofidis, Martinez, GA (US)

Correspondence Address: MUETING, RAASCH & GEBHARDT, P.A., P.O. BOX 581336, MINNEAPOLIS, MN, 55458-1336, US

(73) Assignee: Medical College of Georgia Research Institute Inc.

(21) Appl. No.: 12/744,866

(22) PCT Filed: Jan. 8, 2010
(86) PCT No.: PCT/US10/20446

§ 371 Date: May 26, 2010

Related U.S. Application Data
(60) Provisional application No. 61/204,668, filed on Jan. 10, 2009.
(60) Provisional application No. 61/255,665, filed on Oct. 28, 2009.

(51) Int. Cl. G01N 33/48; A01K 67/033; C12N 5/10; C12Q 1/02; A61K 38/00; A61P 25/28; A61K 38/02; A61P 25/16; A61P 25/00; A61P 35/00; A61K 31/715; A61K 31/7084; A61K 39/395; A61K 35/76

(52) U.S. Cl. 800/3; 800/9; 800/12; 800/10; 800/13; 435/325; 435/29; 514/17.7; 435/6; 514/19.3; 514/54; 514/44 R; 514/44 A; 424/130.1; 424/93.6

(57) ABSTRACT

The invention provides non-human transgenic animals as models of neurodegenerative brain pathology, including, but not limited to, Alzheimer's disease (AD), and cancer. The non-human transgenic animals of the present invention include an exogenous DNA that reduces or eliminates the expression and/or function of a molecular chaperone, including, but not limited to heat shock protein 110 (Hsp1 10) or heat shock protein 70 (Hsp70). These non-human transgenic animals may be used in methods of screening and identifying compounds useful for the prevention and/or treatment of neurodegenerative brain pathology and/or cancer.

### CONTINUING DATA APPLICATION

This application claims the benefit of U.S. Provisional Application Ser. No. 61/204,668, filed Jan. 10, 2009, and U.S. Provisional Application Ser. No. 61/255,665, filed Oct. 28, 2009, each of which is incorporated by reference herein in their entirety.

### GOVERNMENT FUNDING

The present invention was made with government support under Grant Nos. CA062130, CA121951, and CA132640, awarded by the National Institutes for Health, National Cancer Institute. The Government has certain rights in this invention.

### BACKGROUND

The increased prevalence of certain age-associated neurodegenerative diseases is largely attributable to the increase in average life span among individuals who live in industrialized nations. From a patient perspective, diseases such as Alzheimer's, Parkinson's, or amyotrophic lateral sclerosis are feared because of their slow and progressive nature and because there are no effective treatments or cures for these diseases. The economic and social burden of neurodegenerative diseases is huge and growing rapidly. Compared to our understanding of other human diseases, such as cancer and cardiovascular disorders, our knowledge on the mechanisms of neurodegeneration is still in its infancy. Indeed, for most neurodegenerative diseases, we can only guess as to why neurons ultimately die. Alzheimer's disease (AD) is the world's leading cause of dementia and the most prevalent neurodegenerative disease, accounting for an estimated 50-75% of dementia cases. Parkinson's disease (PD) is the world's second commonest neurodegenerative disease. With the world's population living longer, the numbers of sufferers of neurodegenerative diseases are set to rise over the next several decades and the World Health Organization (WHO) calculates that neurodegenerative disease will become the world's second leading cause of death by the year 2040, overtaking cancer.

To alleviate the burden of neurodegenerative diseases on individuals, families, society, and healthcare systems, there is a need for improved therapeutic interventions for these devastating diseases. To accomplish this, there is a need for an improved understanding of the molecular and cellular aspects of the disease mechanisms and of the physiological and pathophysiological functions of cellular proteins that contribute to neurodegeneration. And, there is a need for the identification of potential therapeutic targets and for improved means for evaluating potential therapeutic interventions in model systems.

Further, cancer is a widespread and deadly disease. Although a variety of therapeutic strategies are currently used for treatment of cancer, for many cancers these treatments do not offer a permanent cure for the disease. Significant improvements in the treatment of cancer have proven difficult to develop. Currently, the standard to measure the success of a new anti-cancer drug is often an increase in the survival of cancer patients in terms of months, not in years. There is a need for improved agents for the treatment of cancer.

### SUMMARY OF THE INVENTION

The present invention includes a genetically engineered non-human animal including an exogenous DNA, wherein the exogenous DNA reduces or eliminates function of a molecular chaperone, and wherein the animal is predisposed to brain pathology. In some embodiments, the brain pathology includes a neurodegenerative disease, cognitive disorder, or traumatic brain injury. In some embodiments, the neurodegenerative disease includes amyotrophic lateral sclerosis, Alzheimer's disease, Parkinson's disease, and polyglutamine diseases. In some embodiments, the brain pathology includes aggregation of Aβ and/or hyperphosphorylation p-tau. In some embodiments, the molecular chaperone is a heat shock protein. In some embodiments, heat shock protein is heat shock protein 70 (Hsp70) or heat shock protein 110 (Hsp110). In some embodiments, the animal is a mouse.

The present invention includes a genetically engineered non-human animal including an exogenous DNA, wherein the exogenous DNA reduces or eliminates function of a molecular chaperone, and wherein the animal is predisposed to reduced angiogenesis and/or tumorgenesis. In some embodiments, angiogenesis includes tumor angiogenesis. In some embodiments, tumorgenesis includes chemically induced tumorgenesis. In some embodiments, tumorgenesis includes tumor initiation and/or tumor growth. In some embodiments, the molecular chaperone is a heat shock protein. In some embodiments, heat shock protein is heat shock protein 70 (Hsp70) or heat shock protein 110 (Hsp110). In some embodiments, the animal is a mouse.

The present invention includes a genetically engineered non-human animal including an exogenous DNA, wherein the animal demonstrates reduced or eliminated function of the heat shock protein 110 (Hsp110) and/or reduced or eliminated function of the heat shock protein 70 (Hsp70).

The present invention includes a cell isolated from a genetically engineered non-human animal including an exogenous DNA described herein. In some embodiments, the cell demonstrates a pathology of a neurodegenerative disease, cognitive disorder, or traumatic brain injury. In some embodiments, the neurodegenerative disease is amyotrophic lateral sclerosis, Alzheimer's disease, Parkinson's disease, or polyglutamine diseases. In some embodiments, the isolated cell demonstrates aggregation of Aβ and/or hyperphosphorylation p-tau. In some embodiments, the cell demonstrates reduced angiogenesis and/or reduced tumorgenesis. In some embodiments, tumorgenesis includes tumor initiation and/or tumor growth. In some embodiments, the cell is a neuron, liver, endothelial, or epithelial cell.

The present invention includes a genetically engineered cell including an exogenous DNA wherein the exogenous DNA reduces or eliminates the function of heat shock protein 110 (Hsp110). In some embodiments, the cell demonstrates a pathology of a neurodegenerative disease, cognitive disorder, or traumatic brain injury. In some embodiments, the neurodegenerative disease is amyotrophic lateral sclerosis, Alzheimer's disease, Parkinson's disease, or polyglutamine diseases. In some embodiments, the isolated cell demonstrates aggregation of Aβ and/or hyperphosphorylation p-tau. In some embodiments, the cell demonstrates reduced angiogenesis and/or reduced tumorgenesis. In some embodiments, tumorgenesis includes tumor initiation and/or tumor growth. In some embodiments, the cell is a neuron, liver, endothelial, or epithelial cell.

The present invention includes a genetically engineered cell including an exogenous DNA wherein the exogenous DNA reduces or eliminates the function of heat shock protein 70 (Hsp70). In some embodiments, the cell demonstrates a pathology of a neurodegenerative disease, cognitive disorder, or traumatic brain injury. In some embodiments, the neurodegenerative disease is amyotrophic lateral sclerosis, Alzheimer's disease, Parkinson's disease, or polyglutamine diseases. In some embodiments, the isolated cell demonstrates aggregation of Aβ and/or hyperphosphorylation p-tau. In some embodiments, the cell demonstrates reduced angiogenesis and/or reduced tumorgenesis. In some embodiments, tumorgenesis includes tumor initiation and/or tumor growth. In some embodiments, the cell is a neuron, liver, endothelial, or epithelial cell.

The present invention includes a method for identifying a compound useful for treatment of brain pathology, the method including administering a candidate compound to a genetically engineered non-human animal described herein and evaluating brain pathology developed by the genetically engineered non-human animal; wherein reduced brain pathology in the genetically engineered non-human animal indicates the candidate compound is a compound useful for the treatment of a brain pathology. In some embodiments, the brain pathology of the genetically engineered non-human animal to which the compound is administered is compared to the brain pathology of a second genetically engineered non-human animal to which no compound has been administered; and wherein reduced brain pathology in the treated genetically engineered non-human animal compared to brain pathology in the non-treated genetically engineered non-human animal indicates the candidate compound is a compound useful for the treatment of a brain pathology. In some embodiments, the first animal and second animals are litter mates. In some embodiments, the brain pathology is neurodegenerative disease, cognitive disorder, or traumatic brain injury. In some embodiments, the neurodegenerative disease is selected from the group consisting amyotrophic lateral sclerosis, Alzheimer's disease, Parkinson's disease, and polyglutamine diseases. In some embodiments, brain pathology is evaluated by aggregation of Aβ, p-tau, or a combination thereof. In some embodiments, brain pathology is evaluated by phosphorylation of tau. In some embodiments, brain pathology is evaluated by Hsp70 or Hsp110 expression. In some embodiments, brain pathology is evaluated behaviorally. In some embodiments, the genetically engineered non-human animal is a mouse.

The present invention includes a method of evaluating the efficacy of a compound for use in treating brain pathology, the method including administering a candidate compound to a genetically engineered non-human animal described herein and evaluating brain pathology developed by the genetically engineered non-human animal; wherein reduced brain pathology following administration of the compound indicates that the compound is effective in treating brain pathology. In some embodiments, the brain pathology of the genetically engineered non-human animal to which the compound is administered is compared to the brain pathology of a second genetically engineered non-human animal to which no compound has been administered; and wherein reduced brain pathology in the treated genetically engineered non-human animal compared to brain pathology in the non-treated genetically engineered non-human animal indicates the candidate compound is a compound useful for the treatment of a brain pathology. In some embodiments, the first animal and second animals are litter mates. In some embodiments, the brain pathology is neurodegenerative disease, cognitive disorder, or traumatic brain injury. In some embodiments, the neurodegenerative disease is selected from the group consisting amyotrophic lateral sclerosis, Alzheimer's disease, Parkinson's disease, and polyglutamine diseases. In some embodiments, brain pathology is evaluated by aggregation of Aβ, p-tau, or a combination thereof. In some embodiments, brain pathology is evaluated by phosphorylation of tau. In some embodiments, brain pathology is evaluated by Hsp70 or Hsp110 expression. In some embodiments, brain pathology is evaluated behaviorally. In some embodiments, the genetically engineered non-human animal is a mouse.

The present invention includes a method for identifying a compound useful for treatment of brain pathology, the method including contacting a cell described herein with a candidate compound and evaluating brain pathology developed by the cell; wherein reduced brain pathology in the cell indicates the candidate compound is a compound useful for the treatment of a brain pathology. In some embodiments, the brain pathology of the treated cell is compared to the brain pathology of a cell to which has not been contacted with the candidate compound; and wherein reduced brain pathology in the treated cell compared to brain pathology in the untreated cell indicates the candidate compound is a compound useful for the treatment of a brain pathology. In some embodiments, the brain pathology is neurodegenerative disease, cognitive disorder, or traumatic brain injury. In some embodiments, the neurodegenerative disease is selected from the group consisting amyotrophic lateral sclerosis, Alzheimer's disease, Parkinson's disease, and polyglutamine diseases. In some embodiments, brain pathology is evaluated by aggregation of Aβ, p-tau, or a combination thereof. In some embodiments, brain pathology is evaluated by phosphorylation of tau. In some embodiments, brain pathology is evaluated by Hsp70 or Hsp110 expression.

The present invention includes a method of evaluating the efficacy of a compound for use in treating brain pathology, the method including contacting a cell described herein with a candidate compound and evaluating brain pathology developed by the cell; wherein reduced brain pathology following administration of the compound indicates that the compound is effective in treating brain pathology. In some embodiments, the brain pathology of the treated cell is compared to the brain pathology of a cell to which has not been contacted with the candidate compound; and wherein reduced brain pathology in the treated cell compared to brain pathology in the untreated cell indicates the candidate compound is a compound useful for the treatment of a brain pathology. In some embodiments, the brain pathology is neurodegenerative disease, cognitive disorder, or traumatic brain injury. In some embodiments, the neurodegenerative disease is selected from the group consisting amyotrophic lateral sclerosis, Alzheimer's disease, Parkinson's disease, and polyglutamine diseases. In some embodiments, brain pathology is evaluated by aggregation of Aβ, p-tau, or a combination thereof. In some embodiments, brain pathology is evaluated by phosphorylation of tau. In some embodiments, brain pathology is evaluated by Hsp70 or Hsp110 expression.

The present invention includes a compound identified by a method described herein. In some embodiments, the compound reduces aggregation of Aβ and/or p-tau, inhibits phosphorylation of tau, reduces of eliminates p-tau, induces dephosphorylation of p-tau, and/or induces degradation of p-tau. The present invention includes a method of treating brain pathology in a subject, the method including administering such a compound to the subject.

The present invention includes a method of evaluating brain pathology in a biological sample from a subject, the method including detecting the co-localization of Hsp70 and Aβ and/or the co-localization of Hsp110 and Aβ; wherein the co-localization of Hsp70 and Aβ and/or the co-localization of Hsp110 and Aβ is indicative of brain pathology.

The present invention includes a method of treating brain pathology in a subject, the method including administering to the subject a compound that increases the expression and/or function of Hsp70 or Hsp110. In some embodiments, the compound reduces the aggregation of Aβ and/or p-tau, inhibits phosphorylation of tau, reduces or eliminates p-tau, induces dephosphorylation of p-tau, and/or induces degradation of p-tau.

The present invention includes a method of detecting a neurodegenerative disease in a subject, the method includes detecting a polymorphic variant or a mutation in one or more hsp110 alleles in a nucleotide sample obtained from the patient. In some embodiments, the neurodegenerative disease is amyotrophic lateral sclerosis, Alzheimer's disease, Parkinson's disease, or polyglutamine diseases. In some embodiments, the neurodegenerative disease is presymptomatic.

The present invention includes a method of identifying a compound for altering the expression and/or function of heat shock protein 70 (hsp70), the method including administering a candidate compound to a non-human animal or an isolated cell; and evaluating the expression and/or function of hsp70 in the animal or cell; wherein altered expression and/or function of hsp70 in the animal or cell following administration of the compound indicates that the compound is effective for altering the expression and/or function of heat shock protein 70 (hsp70). In some embodiments, altering the expression and/or function of heat shock protein 70 (hsp70) is reducing the expression and/or function of heat shock protein 70 (hsp70). In some embodiments, altering the expression and/or function of heat shock protein 70 (hsp70) is increasing the expression and/or function of heat shock protein 70 (hsp70). In some embodiments, the compound affects angiogenesis and/or tumorgenesis.

The present invention includes a method for identifying a compound with anti-angiogenic and/or anti-tumorgenic effect, the method including identifying a compound that reduces or eliminates the expression or function of the heat shock protein 70 (Hsp70) in a cell or animal.

The present invention includes a method for evaluating carcinogenicity of a candidate compound, the method including administering the candidate compound to an animal described herein; evaluating a tumor burden attained by the animal; and comparing the tumor burden attained by the animal to a tumor burden attained by a control animal, wherein a higher tumor burden present in the genetically engineered animal compared to the control animal indicates that the candidate compound is a carcinogen.

The present invention includes a method for evaluating carcinogenicity of a candidate compound, the method including administering the candidate compound to a cell described herein; evaluating the cell for transformation; and comparing the transformation of the cell to transformation of a control cell, wherein transformation in the cell compared to the control cell indicates the compound is a carcinogen.

The present invention includes a method for identifying a candidate anti-carcinogenic compound, the method including administering a carcinogenic compound to an animal described herein; further administering the candidate anti-carcinogenic compound to the animal; evaluating a tumor burden attained by the animal; and comparing the tumor burden attained by the treated animal to a tumor burden attained by a control animal, wherein a lower tumor burden present in the treated animal compared to the control animal indicates the compound is an anti-carcinogenic compound. In some embodiments, the compound affects angiogenesis and/or tumorgenesis.

The present invention includes a method for identifying a candidate anti-carcinogenic compound, the method including administering a carcinogen to a cell described herein; administering the candidate anti-carcinogenic compound to the cell; evaluating the cell for transformation; and comparing the transformation of the cell to transformation of a control cell, wherein transformation in the cell compared to the control cell indicates the compound is a carcinogen. In some embodiments, the compound affects angiogenesis and/or tumorgenesis.

The present invention includes a method of affecting angiogenesis and/or tumorgenesis in a subject, the method including administering a compound described herein to the subject.

The present invention includes a method of treating cancer in a subject, the method including administering to the subject a compound that reduces the expression and/or function of Hsp70.

The terms “comprises” and variations thereof do not have a limiting meaning where these terms appear in the description and claims.

Unless otherwise specified, “a,” “an,” “the,” and “at least one” are used interchangeably and mean one or more than one.

### BRIEF DESCRIPTION OF THE FIGURES

FIG. 1 shows targeted disruption of hsp110 gene in mice. FIG. 1A shows a schematic diagram of wild-type hsp110 locus, targeting vector, and the predicted targeted allele following homologous recombination. Exons are indicated by black boxes (E1-E7). The probe used for Southern blotting and the location of the PCR primers 1, 2, and 3 used for genotyping the mice are indicated. LacZ represents β-galactosidase (β-gal) cDNA. Neo represents neomycin cDNA. FIG. 1B shows Southern blotting. BamHI-digested tail DNA (10 μg) from wild-type (+/+) or hsp110 heterozygote (+/−) was hybridized with an external probe to yield the predicted fragments, 15.6 kb for +/+, and 13 kb for mutant hsp110^{+/-}. FIG. 1C shows PCR from tail DNA prepared from +/+, hsp110^{+/-}, or hsp110^{-/-}. Genotyping amplifies fragments of 405 by for the targeted hsp110 allele, and 242 by for the wild-type allele. FIG. 1D shows 30 μg of protein prepared from extracts of +/+ or hsp110^{-/-} brain analyzed by immunoblotting using antibody specific to Hsp110 or β-gal. β-actin is control for loading. FIGS. 1E and 1F show brain tissue sections from hippocampus (E) or cortex (F) of 4 months old male mice (n=3) stained with X-gal to detect hsp110-lacZ expression. Arrows indicate cells expressing β-gal (blue). Sections were counterstained with Eosin. Panel E, Bar=200 μm. Panel F, Bar=500 μm. FIG. 1G shows cultured neurons fixed and stained with X-gal. Arrow indicates hsp110-LacZ expression in a representative hsp110^{-/-} (−/−) neuron (blue). Bat-10 μm. FIG. 1H shows wild-type indicated brain tissue sections of one year old female mice immunostained with no primary antibody (a), or with Hsp110 specific antibody (b and c). Sections were probed with HRP-conjugated secondary antibody (arrows). Tissue sections were counterstained with hematoxylin. Bar=10 μm.

FIG. 2 shows tau is hyperphosphorylated in hsp110^{-/-} brain. FIG. 2A shows fixed and processed sections of hippocampus region of 24 weeks-old wild-type (WT), or hsp110^{-/-} mice (mix gender, n=3-5) immunostained with antibodies to total tau, p-tau, and Cy3 fluorescent-conjugated secondary antibody. Nuclei were stained with DAPI. Bar=20 μm. FIG. 2B shows tissue sections of hippocampal region of wild-type, or hsp110^{-/-} mice (mix gender, n=3-5) at the indicated age groups immunostained for the presence of total tau or p-tau, as in FIG. 2A, and number of positively stained cells per section were quantitated. +/+ and −/− represent wild-type, and hsp110^{-/-} mice, respectively. *p<0.02; **p<0.01. FIG. 2C shows same as in FIG. 2A, with the bar=5 μm.

FIG. 3 shows Hsp110^{-/-} brain tissue exhibits positive immunoreactivity to Alzheimer's-associated tau phosphorylation specific antibody. FIG. 3A shows sections of hippocampal region of 24-weeks-old wild-type (WT) or hsp110^{-/-} mice (mix gender, n=3-5) immunostained with the indicated antibody to p-tau, and Cy3 fluorescent-conjugated secondary antibody. Bar=20 μm. Lower panels show quantitation of the data presented in panel A at the indicated age groups (mix gender, n=3-5 mice). +/+ and −/− represent wild-type and hsp110^{-/-} mice, respectively. *p<0.01. FIG. 3B shows forebrain tissue sections of 24-weeks-old wild-type (WT), or hsp110^{-/-} mice (mix gender, n=3) immunostained with MC1 antibody and Cy3 fluorescent-conjugated secondary antibody. Scale bar=20 μm. Lower panels show quantitation of the data from the indicated age groups (mix gender, n=4 mice). *p<0.04. FIG. 3C shows same as in FIGS. 3A and 3B. Bar=5 μm. In all panels, nuclei were stained with DAPI.

FIG. 4 shows accumulation of soluble and insoluble p-tau in aging hsp110^{-/-} brain. Brain tissue extracts from 4-6, or 24-30 weeks old wild-type (+/+) or hsp110^{-/-} (mix gender, n=3) mice were. FIG. 4A shows homogenized and soluble (S1), Sarkosyl soluble (S2) and Sarkosyl-insoluble (P3) fractions. 30 μg of S1 and 10 μg of S2 fractions were loaded on the gel. For immunoblotting, PHF1 or CP13 antibodies that recognize p-tau were used. β-actin is loading control. FIG. 4B shows S1 fraction of wild-type or hsp110^{-/-} brain tissue (as presented in panel (A)) untreated (-Cip), or treated with CIP (+CIP) for 15 minutes at 30° C. Lysates were then analyzed by immunoblotting using PHF1 antibody to detect p-tau. β-actin is loading control. FIG. 4C shows brain tissue sections from 24-30 weeks-old wild-type (+/+) or hsp110^{-/-} male mice (n=3-5) stained with Bielschowsky to detect NFTs (arrows). Panel (a) represents cortical region of wild-type brain. Panels b-d represent cortical (b, d) and hippocampal brain region \textcircled{c} of hsp110 mice. Bar=10 μm

FIG. 5 shows increased apoptotic cells in hsp110^{-/-} aging brain tissue. FIG. 5A shows brain (hippocampus) tissue sections of 4-6, 10-14, or 24-32 weeks-old from wild-type (+/+) or hsp110^{-/-} mice stained with TUNEL (arrow). Bat-5 μm. Lower panels show quantitation of the data presented in panel A (mix gender, n=4). Arrow indicates an apoptotic cell. *p<0.03; **p<0.009. FIG. 5B shows brain tissue sections (cortex) of 8-months-old wild-type and hsp110^{-/-} mice immunostained with neuron (NeuN)- or astrocytes (GFAP)-specific antibody. Bars=500 μm or 50 μm for upper and lower panels, respectively. Number of immunopositive cells was quantitated (mix gender, n=3) and data are presented in the left panels. *p<0.05. In all panels, nuclei were stained with DAPI.

FIG. 6 shows Hsp110 interacts with tau and Pin1. FIG. 6A shows immunoblotting of wild-type (WT) and hsp110^{-/-} brain extracts to detect the indicated proteins. FIG. 6B shows 1 mg of brain extracts from 7-month-old wild-type (WT) or hsp110^{-/-} mice subjected to immunoprecipitation (IP) followed by immunoblotting to detect Hsp110 (top panel) or Pin1 (lower panel) using appropriate antibodies. Lane 1, IP of WT brain extracts using nonspecific antibody. Lanes 2 and 5, IP of WT or hsp110^{-/-} brain extracts using antibody to total tau (detects both p-tau and tau). Lanes 3 and 4, immunoblot of brain extracts from WT and hsp110^{-/-} mice presented as controls. FIG. 6C shows brain extracts from WT or hsp110^{-/-} mice (as in B) subjected to IP using antibody to Hsp110 or to total tau followed by immunoblotting to detect Pin1. Lane 1, IP of WT brain extracts using nonspecific antibody. Lane 2, IP using antibody to Hsp110 to immunoprecipitate Pin1 from WT brain extracts. Lane 3, immunoblot of Pin1 in brain extracts. Lanes 4 and 5, IP using antibody to total tau (DA9) to pull-down Pin1 from brain extracts of wild-type or hsp110^{-/-} mice. All immunoblots contained 30 μg of proteins. In all panels, β-actin is loading control. For panels A-C, 7 months old mice were used (mix gender, n=3-5). FIG. 6D shows 2 μM of purified full-length Hsp110 mixed with 2 μM of purified tau. Tau was then immunoprecipitated using antibody to tau (DA9) and the mixture was immunoblotted using antibody to Hsp110. First lane represents Hsp110 in brain extracts. Middle lane represents immunoprecipitated tau interaction with Hsp110. Last lane represents negative control where nonspecific antibody was used to pull-down Hsp110. FIG. 6E shows peptidyl prolyl isomerase (PPIase) activity is lower in hsp110^{-/-} brain extracts. PPIase activity was measured in 2 μg of brain extracts from 2 or 7 months old wild-type or hsp110^{-/-} male mice (n=7). *p<0.05, **p<0.001.

FIG. 7 shows Tau is present in immunocomplexes with several Hsps, Chip, and Pin1. One mg of brain lysates from the indicated groups was immunoprecipitated using antibody to total tau and immunoblotting experiments were performed to detect individual Hsps, Pin1, or Chip. Lane 1, indicates protein expression in wild-type brain cell lysate. Lanes 2-7 are brain cell lysates prepared from 6 weeks old (6 w) or 8 months old (8 m) wild-type, hsp110−/−, or hsp70i−/− mice (mix gender, n=3). Lane 8 represents wild-type brain lysate containing nonspecific antibody used as a negative control (-ve). Hsp60 was not detected in these tau immunocomplexes. The amount of immunoprecipitated Chip was 2-fold higher in aged wild-type, hsp110−/−, and hsp70i−/− brain extracts.

FIG. 8 shows Hsp110−/− mice exhibit no deficiency in open field behavioral tests. Wildtype and hsp110−/− mice (n=6-8 male mice) were subjected to Open Field behavioral tests at one year of age.

FIG. 9 shows Hsp110−/− mice exhibit some deficiency in Y-maze/spontaneous alternation behavioral tests. Wild-type (WT) and hsp110−/− mice (n=6-8 male mice) at one year of age were subjected to spontaneous alternation tests. *p<0.05. L and R represent left and right entries, respectively.

FIG. 10 shows Hsp110^{-/-} mice exhibit deficits in contextual fear conditioning test. FIG. 8A represents one year old wild-type and hsp110^{-/-} male mice (n=6-8) subjected to Contextual and Cued Fear Conditioning tests. *p<0.05. FIG. 8B shows one and half years old wild-type and hsp110^{-/-} male mice subjected to hanging bar test and percentage of the mice that fall off during a 45 seconds were determined. *p<0.05.

FIG. 11 shows Hsp70i^{-/-} mice exhibit age-dependent accumulation of p-tau. FIG. 11A shows brain of 28 weeks-old wild-type (WT) or hsp70i^{-/-} mice fixed and processed. Brain sections (hippocampus) were immunostained with the antibodies to total tau (DA9), or p-tau (pS202-T205) (CP-13), or (S262/356) (PHF1) and detected with Cy3 fluorescent-conjugated secondary antibody. Nuclei were stained with DAPI. Bar=5 pith FIG. 11B shows tissue sections of hippocampal region of wild-type or hsp70i^{-/-} mice (mix gender, n=3) at the indicated age groups immunostained for the presence of p-tau as in FIG. 11A and number of positively stained cells per section were quantitated. +/+ and −/− represents wild-type and hsp70i^{-/-} mice, respectively. *p<0.08 (upper panel) and p<0.04 (lower panel); **p<0.01. FIG. 11C shows brains from 32 weeks-old male mice from wild-type (panel a) or hsp70i^{-/-} (panels b-d) mice fixed and processed. Panel a, b and d are cortical region and panel c is from hippocampus region. Sections were stained with Bielschowsky to detect NFTs (arrows). Bar=10 μm (a-c); 5 μm (d).

FIG. 12 shows Hsp110 protein interacts with APP and is involved in Aβ generation. FIG. 12A shows Tg2576⁺ (12 months old) or hsp110^{-/-}Tg2576⁺ (7 months old) brain extracts from male mice (n=3) subjected to immunoprecipitation using antibody to APP. Immunoblotting experiments were performed to detect Hsp110. Lane 1 represents Hsp110 expression level in the wild-type brain extracts. Lanes 2 and 3 represent immunoprecipitation using brain extracts from Tg2576⁺ or hsp110^{-/-}Tg2576⁺ mice. Lane 4 represents immunoprecipitation of Tg2576⁺ brain extracts using a nonspecific antibody as a negative control. Right panel represents immunoblotting experiment to detect APP in the cell lysates. Lanes 1-3 are the same as the left panel. Antibody to APP recognizes human protein only. β-actin is loading control. FIG. 12 B shows brains from Tg2576⁺ (8 months of age), hsp110^{-/-} (6 months of age) or hsp110^{-/-} Tg2576⁺ (6 months of age) male mice (n=3) fixed, processed and stained with Bielschowsky to detect NFTs (arrowheads). Bar=10 μm. FIG. 12C demonstrates, as indicated in the panels, brains from wild-type (12 months of age) and Tg2576⁺ (8 and 12 months of age), hsp110^{-/-} (12 months of age) or hsp110^{-/-}Tg2576⁺ (7 months of age) male mice (n=3) fixed, processed, and stained with Congo Red to detect neuritic plaques (arrows). Bar=10 μm. FIG. 12D shows, as indicated in the panels, brains from Tg2576⁺ (8 and 12 months of age), or hsp110^{-/-}Tg2576⁺ (7 months of age) male mice (n=3) fixed and processed. Sections were immunostained using primary antibody to Aβ and Cy3 fluorescent conjugated secondary antibody to detect neuritic plaques. Bar=10 μm.

FIG. 13 shows Hsp110^{-/-} mice exhibit wild type levels of α and β secratase activities but show an accelerated increase in the insoluble Aβ42 generation. In FIG. 13A, soluble brain extracts of 7 months old male mice (n=4) were used to determine the activities of α and β secretases. Note that hsp110^{+/+}Tg2576+ litter-mates were used as controls for hsp110^{-/-}Tg2576+ mice (female n=3). *p<0.05. In FIG. 13B, soluble and insoluble brain extracts of one year old male mice (n=3-5) were prepared and Aβ40 and Aβ42 levels determined using ELISA. Data presented as ng/g of protein. *p<0.05, **P<0.01. In FIG. 13C, seven month old female +/+, hsp110^{+/-}Tg2576+ and hsp110^{-/-} Tg2576+ insoluble brain extracts were prepared (n=3) and the levels of Aβ40 and Aβ42 determined by ELISA. +/+ and Tg2576+ mice (mix gender, n=3) were one year of age. **p<0.01, ***p<0.001. FIG. 13D shows immunoblotting to detect mutant sAPPβsw species in soluble brain extracts of 6 weeks old, 10, or 6 months old female mice (n=3) of indicated groups have been presented. β-actin is loading control. Note that in all panels, hsp110^{+/-}Tg2576+ mice were used as controls for hsp110^{-/-}Tg2576+ mice. +/+ is negative control in data presented in panel FIG. 13C, since ELISA kits used only recognize human Aβ40 or Aβ42 species. For statistical analyses, one-way analyses of variance (ANOVA) were used.

FIG. 14 shows expression of Hsp110 and amyloid β in healthy and Alzheimer's brain tissue. FIG. 14A shows paraffin-embedded human tissue sections of Alzheimer's brain (Biochain) (hippocampal region) immunostained using primary antibodies to Hsp110, and amyloid β, and Cy3 or FITC fluorescent conjugated secondary antibodies. Panels show amyloid β staining of neuritic plaques (arrows) and Hsp110 staining of neurons arrowheads. FIG. 14B shows paraffin-embedded brain tissue sections (hippocampal region) immunostained using primary antibody to Hsp110 and horseradish peroxidase conjugated or Cy3 fluorescent conjugated secondary antibodies (Hsp110 is dark staining in the cytoplasm, arrows). Bars=10 μm.

FIG. 15 shows a hypothetical model of how Hsp110 affects tau phosphorylation, APP processing, and Aβ generation. Absence of Hsp110, Hsp70, Chip, or Pin1 gene leads to hyperphosphorylated tau in brain tissue.

FIG. 16 shows targeted disruption of hsp70.1 and hsp70.3 (hsp70i) alleles. FIG. 16A is a diagrammatic representation of genomic structure of hsp70.1 and hsp70.3 loci. FIG. 16B is a diagrammatic representation of the targeting vector constructed by replacing approximately 15 kb of genomic DNA, including the start codon of hsp70.3 and stop codon of hsp70.1 alleles, with a neomycin resistance (neo) gene flanked by loxP sequences and LacZ reporter gene. The vector was designed so that the promoter of the hsp70.3 gene drives reporter expression and FIG. 16C shows the predicted targeted allele following homologous recombination. FIG. 16D shows Southern blotting analyses of genomic DNA digested with EcoRI and hybridized with a probe external to the targeting vector (indicated) yields a 12 kb fragment for the wild-type and 9 kb fragment for targeted allele, correspondingly. PCR based genotyping using a combination of P1, P2 and P3 primers amplifies wild type and targeted hsp70.1/3 locus fragments of 469 by and 1001 bp, respectively. The locations of Neo and thymidine kinase (tk) genes, probes for southern blotting and primers for PCR analysis are indicated. Restriction sites are designated: R, EcoRI; B, BamHI; E, EcoICRI. FIGS. 16E and 16F show Hsp70.3 gene expression in tissues of Hsp70.3+/− or Hsp70i+/− mice visualized by staining for LacZ reporter activity. Tissue-specific constitutive hsp70.3 expression in tissues from adult mice (8 weeks old) under normal-conditions (FIG. 16E) or stress-induced expression following whole body hyperthermia (FIG. 16F). Tissues were harvested, frozen, sectioned, fixed and stained with X-gal. Original magnification was 200×.

FIG. 17 shows expression of Hsp70.3 in mice deficient in Hsfs during embryonic development. Embryos from Hsp70.3^{+/-} mice alone (E7.5, E9.5, E12.5; upper panels) or bred on a Hsf1, Hsf2 or Hsf4 deficient genetic background (E9.5; lower panels) were fixed in 0.2% glutaraldehyde for 30 min and permeabilized by detergent rinse. Staining for LacZ was performed with X-gal. Expression of the reporter gene in endothelial cells of embryonic blood vessels (E9.5) was visualized by whole mount staining for CD31, an endothelial cell marker and microscopic evaluation of paraffin sections. Genotyping of the embryos was performed by PCR.

FIG. 18 shows reduced tumor growth in Hsp70^{-/-} mice. FIGS. 18A-18C show expression of Hsp70i visualized by expression of the LacZ reporter gene in transplanted tumors. 1×10⁷ LLC tumor cells were injected subcutaneously into the right flank of Hsp70.1^{+/-} mice. At different developmental stages tumor tissues were fixed, sectioned and stained with X-gal. Endothelial cells lining the lumen of blood vessels at day7 (early stage) (FIG. 18A) expressed hsp70.1-lacZ reporter activity, whereas no such expression was observed in day-14 tumors (late stage) (FIG. 18B). Note that LLC cells have an intact hsp70i locus and do not express lacZ, unequivocally indicating that blood vessels are of host origin. FIG. 18C shows immunostaining for CD31 indicating endothelial cells in blood vessels infiltrating day7 tumor is also presented. FIGS. 18D-18F show reduced tumor growth and density of microvessels in Hsp70i^{-/-} mice. In FIG. 18D (left panel (D1) and right panel (D2)) Hsp70.1^{-/-}, Hsp70.3^{-/-}, Hsp70i^{+/-} (Hsp70.1/3^{+/-}), Hsp70i^{-/-} (Hsp70.1/3^{-/-}) or C57BL/6J mice were inoculated with LLC tumor cells (1×10⁶) and tumor growth was monitored over the period indicated. Data shown are mean±SEM tumor volume of 6 mice. FIG. 18E (left panel) shows numbers of tumor blood vessels for the same experiments is presented for day 7 after tumor implantation. Data are mean±SEM blood vessels per field (400×) of 3 mice. (P<0.001 comparing B6 with Hsp70.1^{-/-}, or Hsp70i^{-/-} (Hsp70.1/3^{+/-}), or Hsp70i^{-/-} (Hsp70.1/3^{-/-}) mice). FIG. 18E (middle and right panels) shows fluorescence microscopy of representative tumor sections (day 7 after transplantation) stained for CD31 revealed significant decreased microvascular density in Hsp70i^{-/-} (middle panel) mice compared to wild-type controls (right panel).

FIG. 19 shows loss of Hsp70.1 or Hsp70.3 prolongs tumor free survival in the p53 deficient mouse model for spontaneous tumorigenesis. FIG. 19A is a Kaplan-Meier analysis of tumor incidence in a cohort of p53^{-/-} (N=16), p53^{-/-}Hsp70.1^{-/-} (N=22) and p53^{-/-} Hsp70.3^{-/-} (N=28) mice. P<0.002 comparing P53^{-/-} with P53^{-/-}70.3^{-/-} or P<0.003 comparing P53^{-/-} with p53^{-/-}70.1^{-/-}). FIG. 19B is a comparison of tumor incidence (tumor spectrum) of p53^{-/-}70.3^{-/-}, p53^{-/-}70.1^{-/-} to a cohort of p53^{-/-} mice. FIG. 19C shows representative histology of hemangiosarcoma; HE-staining (FIG. 19C, left panel (C1)) and X-gal staining (FIG. 19C, middle panel (C2)). FIG. 19D shows representative histology of squamous carcinoma (X-gal staining) from Hsp70.1^{-/-}p53^{-/-} mice. Note strong staining for LacZ reporter activity detecting Hsp70i expression in these tumors (example for the high level of Hsp70 expression in tumors). Original magnification was 200×.

FIG. 20 shows constitutive Hsp expression in mouse endothelial cells. Lysates from lymphomas arising in p53^{-/-} mice (A1, A4), bone marrow cells from C57BL/6 mice (BM), and C166 (C166) cells were analyzed for Hsp-expression by western blotting. As a control for equal protein loading, the blots were probed for β-actin.

FIG. 21 shows imaging brain edema following TBI in mice. FIG. 21A shows magnetic Resonance Imaging (MRI) from one mouse brain section (slice 11) following TBI. Baseline apparent diffusion coefficient mapping (ADC) and T2-weighted (T2W) images were collected prior to TBI (Pre-TBI), followed by imaging at 24 hours, 1 week, and 3 weeks post-TBI. Vasogenic and cellular edema are bright on T2W images, in proportion to the relative volume of liquid. Dark areas on the ADC map indicate cellular edema and bright areas suggest vasogenic edema. By 24 h post-TBI, cellular edema predominates and is associated with some brain herniation. In contrast, vasogenic edema is observed between 1-3 weeks following injury. In FIG. 21B, wild-type male mice (n=5) were subjected to TBI and 24 hours later, brain water content was estimated in a 3 mm coronal tissue section of the ipsilateral cortex (or corresponding contralateral cortex that were not treated), centered on the impact site. Tissues were immediately weighed (wet weight), then dehydrated at 65° C.). The samples were reweighed 48 hours later to obtain a dry weight. The percentage of water content in the tissue samples were calculated using the following formula: {(wet weight−dry weight)/wet weight}×100. *p<0.001.

FIG. 22 shows a model for the cooperation of Hsp110 and Hsp70 in protein folding recruitment of Hsp70 to unfolded substrate protein (such as Tau) assisted by Hsp40 (step 1). Formation of complexes between Hsp70 and Hsp110 displaces ADP from the Hsp70 partner (step 2). Direct substrate binding to Hsp110 could provide an anchor aiding the unfolding of kinetically trapped intermediates (e.g in this case tau) through the peptide binding domain (PBD) of Hsp70. Finally, upon binding of ATP to Hsp70, the Hsp70-Hsp110 complexes dissociate and the substrate protein (e.g., tau) is released for folding (step 3). The circle indicates natively folded substrate protein (N). The designations “A”-“E” indicates the possible scenario that p-tau binds to Hsp110 (“A”); and this triggers Hsp110 to bind Hsp70 (“B”); Hsp70/Hsp110/p-tau recruits Pin1 for isomerization and dephosphorylation of p-tau by PP2A (“C”); causing the release of unphosphorylated tau from Hsp110 (“D”); and thereby releasing tau to bind to microtubules (MT) (“E”). This cycle of p-tau isomerization & dephosphorylation requires the activity of Hsp110 and Hsp70.

FIG. 23 shows a model for Hsp90 and Hsp70i in suppression of neurotoxicity. Stage “A” represents normal physiological pathway for tau phosphorylation/dephosphorylation, requiring Hsp70/Hsp110. In “B,” tau may be transferred to protein complexes where it is degraded. As shown in “C,” under disease conditions, p-tau dephosphorylation and degradation may slow down resulting in accumulation of the complexes.

FIG. 24 shows a simplified schematic model for the function of Hsp70i/Hsc70 and Hsp25 in vasculogenesis and angiogenesis under investigation in this specific aim. FIG. 24A shows a scenario in which Hsp70i, Hsc70 and Hsp25 act in a cytoprotective manner promoting blood vessel formation via inhibition of general apoptosis pathways and interfering with key apoptotic protein expression induced by heat shock or other stress situations in endothelial cells. FIG. 24B shows a second scenario, in which these Hsps act either in stabilizing HIF-1 activity (Hsp70i, Hsc70) and/or regulating endothelial cell migration (Hsp25).

FIG. 25 shows that Hsp70i deficiency inhibits DEN-induced tumorigenesis. 15-day-old male mice were injected intraperitoneally with a single dose of DEN (25 g/Kg). Seven months after DEN administration, hsp70i^{+/+} controls (B6) (n=8) or hsp70i^{-/-} (n=15) mice were euthanized and the number and sizes of tumors were quantitated. Results are mean+/−standard deviation for each group. p<0.0001.

FIG. 26 shows representative images of liver tumors in Hsp70i^{+/+} (wild-type) compared to Hsp70^{-/-} mice, which were almost free of tumors. In FIGS. 26A and 26B, seven months after DEN administration, hsp70i^{+/+} (FIG. 26A) or hsp70^{-/-} (FIG. 26B) mice were euthanized, livers were removed and tumors were recorded. Arrows indicate tumors (magnification 2×). FIGS. 26C and 26D show H&E staining of liver sections of 7-month-old DEN treated hsp70i^{+/+} (FIG. 26C) or hsp70i^{-/-} (FIG. 26D) mice (magnification 200×). FIGS. 26E and 26F show liver sections of hsp70i^{+/+} (FIG. 26E) or hsp70i^{-/-} (FIG. 26F) mice were stained with oil red O. Lipid deposits are detected as dark areas in FIG. 26E.

FIG. 27 shows ALT level in serum was determined seven months after DEN administration (DEN) (n=5-15 mice/group) using standard assays. Results are mean+/−standard deviation for each group. p<0.0075 for hsp70i^{+/+} versus hsp70i^{-/-} mice. As a control the ALT activity in serum of age-matched untreated male mice was measured.

FIG. 28 shows Hsp25 deficiency inhibits DEN-induced tumorigenesis. Fifteen day-old male mice were injected intraperitoneally with a single dose of DEN (25 mg/kg). Seven months after DEN administration, hsp25^{+/+} controls (B6) (n=10) or hsp25^{-/-} (n=10) mice were euthanized and number of tumors were quantitated. Results are mean+/−standard deviation for each group. p<0.054.

FIG. 29 shows a profile of proliferating and apoptotic cells following DEN administration in 15 days of age Hsp70i^{+/+} or hsp70i^{-/-} mice. In FIG. 29A, serum ALT levels were determined at 0 hr (no DEN) or 24 and 48 hours after DEN administration (n=3 mice/group) using standard assays (Pointe Scientific Inc). In FIG. 29B, the number of proliferating cells (ki67) was determined by Ki-67 antigen staining on liver sections at 0 hr (no DEN) or 24 and 48 hrs after DEN. In FIG. 29C, apoptotic cells were determined by TUNEL assay on liver sections from Hsp70i^{+/+} (panel a and b) or Hsp70i^{-/-} mice \textcircled{c} and d). TUNEL-assay was performed at 0 hr (no DEN) (panel a and c) or 24 hrs after DEN administration (panel b and d). In FIG. 29D, the number of apoptotic cells per liver section (n=3 mice) was quantitated and results are expressed as mean+/−standard deviation for each group. No significant differences in the number of apoptotic cells in the liver of both genotypes were detected.

FIG. 30 shows cytokine expression in livers of wild-type and hsp70i^{-/-} mice following DEN administration. Mice were treated with DEN at 15 days of age and liver RNA was extracted at the indicated times. Levels of cytokine mRNA were determined by semiquantitative PCR. Results are means+/−standard deviation for a group of three mice.

FIG. 31 shows Hsp70i deficiency inhibits chemical-induced skin tumorigenesis. FIG. 31A shows lower skin tumor incidence and lower tumor burden in Hsp70i^{-/-} mice. Results are means+/−standard deviation for a group of ten male and ten female mice. FIGS. 31B and 31C shows expression of Hsp70i or Hsp25 in the skin visualized by staining for reporter β-galactosidase activity (LacZ staining). Hsp70^{+/-} β-gal or Hsp25^{+/-13}-gal reporter mice were used for this analysis.

FIG. 32 is a schematic demonstrating the potential role for Hsps in DEN-induced HCC.

FIG. 33 outline the experimental protocol to examine the role of Hsf1 in liver tumors. Development of tumors will be initiated at 2 weeks of age (arrows) by a single injection of DEN, and Hsp25 or Hsc70 ablation will be achieved by injection of polyI/C (indicated by *) at different times during the course of the disease to induce expression of cre (Mx1-cre). This experiment will examine the role of Hsps in tumor formation (initiation, promotion and progression) (FIG. 33A), tumor promotion (FIG. 33B), and tumor progression (regression) (FIG. 33C). The extent of metastasis to the lung in mutant mice will be examined in each protocol.

### DETAILED DESCRIPTION OF ILLUSTRATIVE EMBODIMENTS OF THE INVENTION

The present invention demonstrates that deficiencies in the heat shock proteins Hsp110 and/or Hsp70 lead to brain pathology characterized by the accumulation of hyperphosphorylated tau and neurofilament formation. This brain pathology is characteristic of the neurodegeneration observed in a number of neuropathologies including, but not limited to, Alzheimer's disease, Parkinson's disease, and other taupathies, and traumatic brain injury (TBI).

The present invention includes an animal model for the brain pathologies of neurodegenerative diseases. The animal is a genetically engineered non-human animal in which expression of a particular molecular chaperone has been reduced or eliminated and the animal is predisposed to brain pathologies. The present invention shows, for the first time, the presence of both neurofibrillary tangles (NFT) and senile plaques in an in vivo animal model. In some aspects, the animal models for brain pathology of the present invention include a genetically engineered non-human animal in which expression of the molecular chaperones Hsp110 and/or Hsp70 have been reduced or eliminated and the animal is predisposed to brain pathologies.

The present invention also demonstrates that deficiencies in the heat shock protein Hsp70i result in the inhibition of angiogenesis and/or the inhibition of tumorgenesis. In some aspects, angiogenesis includes tumor angiogenesis. In some aspects, tumorgenesis includes tumor initiation and/or tumor growth. The present invention includes an animal model for angiogenesis and/or tumorgenesis. The animal is a genetically engineered non-human animal in which expression of a particular molecular chaperone has been reduced or eliminated and the animal demonstrates the inhibition of angiogenesis and/or the inhibition of tumorgenesis. In some aspects, the animal models for angiogenesis and/or tumorgenesis of the present invention include a genetically engineered non-human animal in which expression of the molecular chaperones Hsp110 and/or Hsp70 have been reduced or eliminated.

Also included in the present invention are methods of using such animal models. For example, the present invention includes, without limitation, methods of screening for agents effective in modulating brain pathologies, methods of evaluating the efficacy of agents used in treating brain pathologies, methods of treating brain pathologies, and methods of identifying genetic variations associated with a predisposition to develop a neurodegenerative disease. For example, the present invention includes, without limitation, methods of screening for agents effective in modulating angiogenesis and/or tumorgenesis, methods of evaluating the efficacy of agents used in effecting angiogenesis and/or tumorgenesis, methods of treating a subject for pathologies in angiogenesis and/or tumorgenesis, and methods of identifying genetic variations associated with a angiogenesis and/or tumorgenesis.

The present invention demonstrates that molecular chaperones, including Hsp110 and Hsp70i, are important in brain pathology, angiogenesis, and tumorgenesis. Heat shock proteins (also referred to herein as “Hsps,” “hsp,” “HSPS,” “HSPS,” and “HSP”) are molecule chaperones that play an important role in protein folding, refolding, assembly, and degradation. Heat shock proteins are classified into several families on the basis of their apparent molecular weights: Hsp110, HSp90, Hsp70, Hsp60, Hsp40, and small Hsps (25 to 28 kDa) (See, for example, Lindquist and Craig, 1988, Annu Rev Genet; 22:631-677).

With the present invention, enhancing the expression level of Hsps early during neurodegenerative disease or after traumatic brain injury may reduce neuronal death, the development of NFTs and/or the cognitive decline in neurodegenerative disease or following traumatic brain injury. This invention represents a significant advancement towards an understanding of brain pathology and the underlying mechanisms of both neurodegenerative disease and traumatic brain injury. The present invention also demonstrates that molecular chaperones, including Hsp70i complexes, are important in regulating angiogenesis and tumorgenesis.

Both human and animal studies indicate that traumatic brain injury (TBI) leads to increased Aβ production in the short-term, and the risk of developing AD, including NFTs, diffuse amyloid plaques, and/or tau immuno-reactivity, in the long-term. TBI is prevalent in accidents and combat situations. For example, two-third of injured US soldiers that are sent to Walter Reed Army Medical Center are diagnosed with TBIs. Studies have found long-term cognitive decline during the years following recovery from TBI compared to controls, with differences in cognitive decline became more significant with age. Corkin and Sullivan examined the penetrating head injury survivors from World War II and discovered that head injury significantly contributed to the cognitive decline of normal aging (Corkin and Sullivan, 1984, J. Gerontol; 39(6):718-20). Some studies have also indicated that the risk of developing dementia increases as the severity of the injury increases. Aβ production can be detected in approximately 30% of patients who die following closed head injury.

The function of molecular chaperones in protein folding includes binding of Hsp70i (or the constitutively expressed Hsc70) to newly synthesized polypeptides or misfolded proteins to facilitate proper folding that requires co-chaperone Hsp40. Hsp70i/Hsc70 and Hsp90 also interact with the ubiquitin E3 ligase CHIP, which facilitates degradation of specific proteins such as p-tau. Hsps also interact with many cellular components, including the cytoskeleton (microtubules, microfilaments, and intermediate filaments).

The function of Hsp110 is less clearly understood. Eukaryotes possess a class of proteins in the cytoplasm known as the Hsp100 family that are related to the Hsp70 family of proteins. The Hsp100 family contains Hsp110 (or Hsp105 in mice, also known as HSPA4) and Apg1 and Apg-2, which are located in the cytoplasm of mammalian cells. The Hsp110 family was initially considered to consist of “holdases” functioning to keep denatured proteins in solution, and no client proteins have been described for them. Hsp110 has been shown to interact with Hsp70 and increase its ATPase activity in the presence of Hsp40 (a co-chaperone of Hsp70). Hsp110 is a phosphorylated protein, is expressed at high levels in neurons and at low levels in other tissues, and it is stress-inducible. The Hsp110 contains an N-terminal ATPase domain, a beta sandwich (domain B) “peptide-binding” domain, and helix domain (domain H) in the C-terminal portion of the protein.

Recent evidence using the crystal structures of Hsp110 and Hsp70 suggests that Hsp110 is a nucleotide exchange factor for Hsp70i. Hsp110 and Hsp70i bind the substrate (e.g., p-tau), leading to a conformational change of p-tau through its interaction with peptidyl prolyl isomerase Pin1 and protein phosphatases (such as PP2A), leading to isomerization and dephosphorylation of p-tau.

The present invention demonstrates, both in vivo and in vitro, the role of Hsp110 in tau phosphorylation state and Hsp110 interaction with tau and Pin1. The present invention also demonstrates that in addition to hsp110^{-/-} mice, hsp70i^{-/-} mice also exhibit an age-dependent development of p-tau, suggesting that depletion of the individual components of the Hsp110-Hsp70i molecular chaperone machine are involved in tau modification. In addition, the present invention demonstrates that Hsp110 interacts with amyloid precursor protein (APP) and that it appears to be involved in APP processing. A number of polymorphic variants of Hsp110 have been found in humans, including a valine to isoleucine polymorphism in amino-acid position 288 (ATP binding domain) as well as seven other polymorphic variants (data from SNP linked to gene, ncbi.nlm.nih.gov/, gene ID:3308). These polymorphisms may play a role in the stability of microtubules or in maintaining tau's proper phosphorylation state, which could cause long-term alterations in the amount of p-tau remaining in cells in individuals carrying such variants.

In general, Hsp110, similar to other Hsps, has been known to induce suppression of aggregation and protein refolding, but no substrate has been yet identified for Hsp110. Hsp110 also protects proteins from the damaging effects of various stresses; however its molecular as well as its physiological function in mammalian cells remains largely unknown. Hsp110 and Hsp70 crystal structures reveal a tight cooperativity between these two molecular chaperones and how they may facilitate the modifications of their substrates. These recent X-ray crystallography data has placed us in a unique position to design critical experiments both in vivo, and neuronal cultures established in vitro, to investigate the role of these proteins on the state of tau phosphorylation and dephosphorylation.

The present invention includes a genetically engineered non-human animal including an exogenous DNA, wherein the exogenous DNA reduces or eliminates the function of a molecular chaperone. In some aspects, the molecular chaperone is a heat shock protein, including, but not limited to Hsp70 or Hsp110. As used herein, the term “genetically engineered” refers to a non-human animal that has induced mutations, including, but not limited to, targeted mutations (knockouts and/or knockins), transgenes, and retroviral, proviral, or chemically-induced mutations. A number of non-human animals are commonly used in genetically engineering animals including, but not limited to, mammals, fruit flies, nematodes, and fish. Preferably, the non-human animal is a mammal. More preferably, the animal is in the order Rodentia. More preferably, the animal is in the family Muridae, such as rat or mouse. Even more preferably, the animal is a mouse.

Typically, genetically engineered non-human animals carry an exogenous DNA which has been incorporated into their genome, for example, via homologous recombination in stem cells, via pronuclear microinjection and non-homologous recombination, or via infection with a retroviral vector. “Exogenous” refers to foreign DNA, that is, DNA that is not normally present in that animal, or DNA that is normally present in that animal but is operably linked to a regulatory sequence to which it is not normally operably linked.

Genetically engineered animals carrying the exogenous DNA on a single allele are referred to as “hemizygous” or “heterozygous” animals. The terms hemizygous and heterozygous are used interchangeable herein. Genetically engineered animals carrying the exogenous DNA on both alleles are referred to herein as “homozygous” animals. Animals that do not carry the exogenous DNA are referred to herein as “wildtype” animals.

In some embodiments, the genetically engineered non-human animal of the present invention is a targeted animal such that the exogenous DNA is inserted into a particular location. The exogenous DNA for use in engineering a targeted animal may be referred to by a number of interchangeable terms including, but not limited to, a “targeting vector,” a “targeting construct,” a “knock-out cassette,” a “targeting cassette,” a “cassette,” and/or a “knock-out vector.” Exogenous DNAs described herein may be prepared using standard recombinant DNA technology and cloning techniques which are well known in the art.

With the present invention, exogenous DNA may be inserted into a gene that encodes a molecular chaperone. A molecular chaperone is a protein that assists in the non-covalent folding/unfolding and the assembly/disassembly of other macromolecular structures. Thus, the molecular chaperone ensure that the cell's proteins are in the proper confirmation in the proper cellular location at the proper time. The molecular chaperone typically is not present in the assisted structure when the latter is performing its normal biological functions. Examples of functions in which molecular chaperone proteins may assist include, without limitation, helping new or distorted proteins fold into shape (which is essential for protein function), shuttling proteins from one compartment to another inside the cell, transporting old proteins for degradation inside the cell, and preventing both newly synthesized polypeptide chains and assembled subunits from aggregating into structures that are nonfunctional or structures exhibiting altered function. More preferably, the exogenous DNA is inserted into a molecular chaperone that assists in preventing aggregation of proteins.

Examples of molecular chaperones include, without limitation, binding immunoglobulin protein (BiP), calnexin, calrecticulum, protein disulferase isomerase (PDI), peptidyl prolyl cis-trans-isomerase (PPI), and the heat shock proteins (Hsps). Preferably, the exogenous DNA is inserted into a gene that encodes a heat shock protein. Hsps are a class of functionally related proteins whose expression is typically increased when cells are exposed to elevated temperatures or other stress. Examples of Hsps include, without limitation, the Hsp70 family, the Hsp90 family, the Hsp27 family, or the Hsp110 family. The Hsp110 family consists of Hspa41 (Apg1 or OSP94), Hsp94 (Apg2), and Hsp110. Hsp110 interacts with Hsp70 and increases its ATPase activity (Easton et al., 2000 Cell Stress Chaperones 5:276-290; Polier et al., 2008 Cell 133:1068-1079; Schuenliann et al., 2008 Mol Cell 31:232-243). The main function of Hsp110 appears to be a nucleotide exchange factor (NEF) for Hsp70 (Dragovic et al., 2006 EMBO J. 25:2519-2528; Shaner et al., 2006 Biochem 45:15075-15084). In general, Hsp110 is known to induce suppression of aggregation, protein refolding, and protects proteins from the damaging effects of various stresses; however, its physiological function in mammalian cells remains unknown (Easton et al., 2000 Cell Stress Chaperones 5:276-290; Saito et al., 2007 Exp Cell Res 313:3707-3717).

In one embodiment, the exogenous DNA is inserted into at least one gene that encodes a member of the Hsp110 family. Preferably, the exogenous DNA is a targeting construct that is inserted into the hsp110 genomic locus.

In another embodiment, the exogenous DNA is inserted into at least one gene that encodes a member of the Hsp70 family. Preferably, the exogenous DNA is inserted into the hsp70i locus containing both the hsp70.1 and the hsp70.3 genes as well as the intergenic region.

In some embodiments, the present invention includes a genetically engineered non-human animal with an exogenous DNA inserted into at least one gene that encodes a member of the Hsp110 family and an additional exogenous DNA inserted into at least one gene that encodes a member of the Hsp70 family.

A targeting cassette for use in engineering the non-human animal of the present invention may include “homology arms” present at both the 5′ and 3′ ends of the cassette. The homology arms are used to direct the exogenous DNA to a particular location by homologous recombination and thereby control the site of genomic insertion, also referred to as the target site. The targeting cassette may be engineered in a vector. Suitable vectors include, without limitation, cloning vectors, expression vectors, yeast artificial chromosomes, bacterial artificial chromosomes, and any other construct that may be used for standard nucleic acid cloning techniques.

Preferably, the cassette may include a long segment of genomic DNA (gDNA) for the 5′ homology arm and a long segment of gDNA for the 3′ homology arm. While longer homology arms often provide more accurate targeting specificity, the length of the homology arm is often limited by vector. Typical homology arms may have a preferred length of about 7 kilobases (kb) combined. For example, the 5′ arm may be about 1 kb while the 3′ arm may be about 6 kb, the 5′ arm may be about 3 kb while the 3′ arm may be about 4 kb, the 5′ arm may be about 3.5 kb while the 3′ arm may be about 3.5 kb, or the 5′ arm may be about 5 kb while the 3′ arm may be about 2 kb. Preferably, the 5′ homology arm is 5-6 kb and the 3′ homology arm is 1-2 kb.

Homology arms may be cloned from genomic DNA of the animal being targeted. For example, the animal being target may be a mouse. The homology arms may be cloned from genomic DNA of the mouse strain to be targeted. The homology arms are cloned from genomic DNA of embryonic stem (ES) cells from the mouse strain being targeted.

A preferred targeting cassette may include a 2.5 kilobase homology arm at the 3′ end and a 5 kilobase homology arm at the 5′ end. In some embodiments, a targeting cassette may include a 2.5 kilobase homology arm cloned from the hsp110 gene at the 3′ end and a 5 kilobase homology arm cloned from the hsp110 gene at the 5′ end.

In some embodiments, the targeting cassette further includes at least a coding sequence. As used herein the terms “coding sequence” and “coding region” refers to a polynucleotide that encodes an RNA, and under the proper regulatory components translates the encoded RNA. A coding sequence may be a genomic sequence, inclusive of non-coding regions (introns) as well as coding regions (exons), or a sequence in which the non-coding portions have been removed, for example a complementary DNA (cDNA). The boundaries of a coding sequence are generally determined by a translation start codon at its 5′ end and a translation stop codon at its 3′ end. Optionally the exogenous DNA further comprises a regulatory sequence. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, transcription terminators, splice signals, introns, and poly(A) signals. Types of promoters may include, without intending to be limiting, constitutive promoters, tissue-specific promoters, inducible promoters, repressible promoters, or leaky promoters. The term “operably linked” refers to a juxtaposition of components such that they are in a relationship permitting them to function in their intended manner.

In some embodiments, the coding sequence encodes a selectable marker. Typically, the coding sequence falls between (i.e. is flanked by) the homology arms and expression of the coding sequence may be used to confirm the presence of the targeting cassette. The selectable marker may provide positive selection such that expression of the selectable marker confers viability under selection conditions. The selectable marker may provide drug resistance. For example, the positive selection marker will confer drug resistance such that only cells harboring the targeting cassette can be viably grown in the presence of selectable media containing otherwise toxic drug. The positive selectable marker may be encoded by an antibiotic resistance gene. For example, the antibiotic resistance gene may include, but is not limited to, a neomycin resistance gene (selectable by G418 or geneticin), a hygromycin resistance gene, a puromycin resistance gene, a kanomycin resistance gene, a blasticidin resistance gene, or a 6-thioguanine gene (selectable by HAT media containing hypoxanthine, aminopterin, and thymidine).

Optionally, the targeting cassette may be engineered such that the selectable marker may be removed. Selectable markers, even when present in non-coding regions such as introns, can have unpredictable effects on the expression of the gene of interest, for example, via inappropriate mRNA splicing events. Methods of excising DNA, whether from a construct or from within the genomic DNA of a non-human animal, are well known in the art and include, for example, using a known recombinase. For example, a coding sequence flanked by loxP sites (i.e. a “floxed” coding sequence) can be removed or excised from the nucleic acid fragment when exposed to the Cre recombinase. In vitro excising may be performed by proving Cre recombinase by any method known in the art. For example, a Cre recombinase coding sequence may be provided on and expressed by an expression vector. Alternatively, Cre recombinase may be provided as a recombinant protein. In vivo excising may be performed by, for example, crossing a genetically engineered animal harboring a cre transgene with a genetically engineered animal harboring an exogenous DNA that includes a floxed coding sequence. Likewise, recombination may be induced by flanking the excisable coding sequence with FRT sites and exposing the targeting cassette to Flp recombinase.

While a targeting construct is designed to integrate into a specific location, non-specific integration may occur. Any integration event, random or specific, can confer drug resistance to the cell by means of a positive selectable marker. After growing the transfected cells under selection, enough clones must be screened to find the rare homologous recombination events in a background of frequent random integrants. Optionally, the exogenous DNA may contain a coding sequence that expresses a second selectable marker. A second selectable marker may provide negative selection such that expression of the selectable markers induces toxicity to cells and/or animals rendering them non-viable in presence of selectable media. Non-limiting examples of coding sequences that confer negative selection include, but are not limited to, thymidine kinase (Tk; selectable with gangcyclovir), adenine phosphoribosyl transferase (APRT) or hypoxanthine (HPRT) selectable with HAT media, or cytidine deaminase (codA) selectable with 5-fluorocytosine.

A coding sequence expressing a negative selection marker on the targeting cassette may be located outside of the homology arms. Therefore, in cases of specific homologous recombination the negative selection marker is not integrated into the genome and growth in selectable media has no effect. However, in cases of non-homologous recombination, the negative selection marker is integrated into the genome and, when expressed, confers toxicity. Optionally, the targeting cassette may include two copies of negative selection gene; one outside of the 5′ homology arm and one outside of the 3′ homology arm.

Advantageously, the exogenous DNA may further include a coding sequence that encodes a reporter. A reporter may be any coding sequence whose expression can be tracked spatially, temporally, or both spatially and temporally. Exemplary reporters include, without limitation, the lacZ gene, fluorescent proteins, and luciferase genes. The reporter may be a lacZ gene. More advantageously, the reporter is in-frame with and expressed by endogenous/native regulatory elements of the targeted gene, thereby enabling one to track the normal, endogenous expression (both spatial and temporal) of the targeted gene as well as alterations of expression under experimental conditions.

Many genes have multiple functions, or are active in multiple tissues and/or at multiple stages of development. Optionally, the exogenous DNA may be engineered such that the targeting construct is a conditional targeting construct. For example, the targeting construct may include recombinase recognition sequences (e.g. loxP sites for Cre recombinase or FRT sites for Flp recombinase), tissue-specific or developmentally-specific promoters, or inducible promoters (or a combination of these) to limit and control the spatial and temporal expression of the exogenous DNA.

The exogenous DNA may further include at least one regulatory sequence. An example of a regulatory sequence is a promoter. A coding sequence may be operably linked to a promoter which drives expression of the coding sequence in a particular manner. The promoter may be linked to a selectable marker. The promoter may drive expression of the coding sequence in, for example, a time-dependent manner, a tissue-specific manner, an inducible manner, a repressible manner, or a constitutive manner. Each coding sequence present in the exogenous DNA may independently contain an independent promoter. The promoter may be a constitutive promoter. A constitutive promoter drives expression of the coding sequence in a continuous and unregulated manner such that expression may be found in all tissues and at all times. Examples of promoters that drive expression of the coding sequence in constitutive manner include, but are not limited to, a thymidine kinase (tk) promoter, a simian virus 40 (SV40) promoter, an actin promoter, a myosin promoter, a human growth hormone (hGH), bovine growth hounone (bGH), and a cytomegalovirus (CMV). Preferably the promoter is a tk promoter.

Optionally, the exogenous DNA may further include a second regulatory sequence, such as a polyadenylation (polyA) signal. Inclusion of a polyA signal increases the stability of the transcript encoded by an exogenous DNA thereby increasing expression. PolyA signals from a number of different genes are commonly used in genetically engineered animals. Each coding sequence present in the exogenous DNA may independently contain a polyadenylation signal. Some commonly used polyA signals include, but are not limited to, polyA signals selected from hGH, SV40, and rabbit β-globin. In some embodiments, the polyA signal may be from an SV40 gene or a bGH gene.

Optionally, the exogenous DNA may further include a third regulatory sequence, such as an intron and the upstream and downstream endogenous splice donor and splice receptor sites. Inclusion of an intron increases the likelihood of the transcript encoded by an exogenous DNA being processed (i.e. spliced) and further translated thereby increasing expression. Introns from a number of different genes are commonly used in genetically engineered animals. Some commonly used introns include, but are not limited to, introns selected from the hGH gene, the SV40 gene, and the rabbit β-globin gene.

In some instances, the exogenous DNA is inserted into the genome in a concatamer. Concatamers are covalently linked tandems of multiple copies of the exogenous DNA. The multiple copies of the exogenous DNA will typically align in a head-to-tail fashion. However, the copies of the exogenous DNA may also align in a head-to-head, in a tail-to-tail fashion, or a combination thereof. The number of copies of the exogenous DNA within the concatamer may vary. The copies may be full copies of the exogenous DNA, partial copies of the exogenous DNA, or a combination thereof. Partial copies of the exogenous DNA may include any portion of the exogenous DNA. For example, the partial copy may include the 5′ end, the middle, or the 3′ end of the exogenous DNA.

The exogenous DNA, when inserted into the genomic DNA, may result in the deletion of a portion of the genomic DNA.

In one embodiment, the genetically engineered animal of the present invention is a hsp110 knock-out (KO) animal, including, but not limited to, a hsp110 knock-out (KO) mouse. For example, a hsp110 KO mouse may include a 3.5 kilobase targeting cassette having a lacZ gene with a bGH polyA signal under the control of the endogenous hsp110 regulatory elements and a floxed neomycin with a tk polyA signal under the control of a tk promoter. In some embodiments, the targeting cassette deletes exon 1 of hsp110 at the ATG start site and 910 base pairs of intron 1 and replaces them, in frame, with the 3.5 kilobase lacZ-neomycin cassette using a 2.5 kilobase insert of hsp110 at the proximal end and a 5 kilobase insert of hsp110 at the distal end. In some embodiments, β-Galactosidase (expressed by the lacZ gene) is detected in the hippocampus and the cortical region, in the coritcal neurons, and in the Purkinje cells of the cerebellum and the cortical neurons of a knock out mouse. A hsp110 KO animal of the present invention may be predisposed to an age-dependent accumulation of p-tau and/or pathological features such as cellular apoptosis and appearance of NFTs. A hsp110 KO animal may also be predisposed to accelerated pathology as evidenced by the early appearance of senile plaques.

In another embodiment, the genetically engineered animal of the present invention is a hsp70i knock-out (KO) mouse. For example, a hsp701 KO mouse may include a targeting cassette having a lacZ gene and a floxed neomycin gene. In some embodiments, the targeting cassette deletes approximately 15 kb of genomic DNA, including the start codon of hsp70.3 and the stop codon of hsp70.1 and replaces them, in frame, with the lacZ-neomycin cassette such that expression of lacZ is driven by the endogenous hsp70.3 promoter. In some embodiments, β-Galactosidase (expressed by the lacZ gene) is detected in the chorionic plate (site of vasculogenesis) at embryonic stages and in the vasculature. A hsp110 KO animal may also be less susceptible to microvessel proliferation (angiogenesis) and tumorigenesis. A hsp70i KO animal of the present invention may be less susceptible to liver or skin carcinogenesis.

The present invention includes progeny of the transgenic animals described herein. Such progeny may be obtained by mating the transgenic mammal with a suitable partner or by in vitro fertilization using eggs and/or sperm obtained from the transgenic mammal. Where in vitro fertilization is used, the fertilized embryo is implanted into a surrogate host or incubated in vitro or both. Where mating is used to produce transgenic progeny, the transgenic mammal may be back-crossed to a parental line, otherwise inbred or cross-bred with mammals possessing other desirable genetic characteristics. The progeny may be evaluated for the presence of the transgene using methods described above, or other appropriate methods. The progeny may include one or more functional characteristics described herein.

Although the foregoing discussion has been made with reference to several methods for producing transgenic mammals, it will be understood that the present invention is not predicated on, or limited to, any one of these methods but instead contemplates any suitable means for producing genetically modified mammals whose germ cells or somatic cells contain a transgene as broadly described above.

The transgenic animals of the present invention can be crossed with animals having other genetic modifications. For example, in some embodiments, a genetically engineered non-human animal of the present invention may further include an exogenous coding sequence for an amyloid-β protein or an amyloid precursor protein (APP).

Amyloid-β protein (also referred to as “amyloid-beta polypeptide,” “Aβ protein,” “Aβ polypeptide,” “Aβ peptide,” “Aβ molecule,” “amyloid-beta,” “amyloidβ,” or “Aβ,” is the major constituent of amyloid plaques in the brains of individuals afflicted with Alzheimer's disease, a polypeptide of 39 to 43 amino acid residue first identified by Glenner and Wong (see, for example, Glenner et al., 1984, Biochem Biophys Res Commun 120:885-890 and Glenner and Wong, 1984, Biochem Biophys Res Commun 122:1131-1135), Masters et al. (see Masters et al., 1985, Embo J 4:2757-2764 and Masters et al., 1985, Proc Natl Acad Sci USA 82:4245-4249). The Aβ peptides are the major amyloid protein deposited in AD brains and both natural and synthetic forms have devastating effects on the viability and function of neurons. See, for example, Yankner et al., 1990, Science 250: 279-82; Pike et al., 1991, Brain Res 563:311-4; Pike et al., 1993, J Neurosci 13:1676-87; Lambert et al., 1998, Proc Natl Acad Sci USA 95:6448-53; Walsh et al., 2002, Nature 416:535-9; and Kayed et al., 2003, Science 300:486-9. The gene for the amyloid precursor protein (APP) of the amyloid-beta protein has been cloned and sequenced (see, for example, Kang et al., 1987, Nature 325:733; Tanzi et al., 1987, Science 235:880-884; and Selkoe, 1994, Annual Review of Neuroscience 17:489-517). As used herein, an amyloid-beta protein may include any of the various known allelic variants and mutations of the amyloid-beta protein.

For example, if the animal of the present invention is a mouse it can be crossed to a mouse carrying the Tg(APPSWE)2576 Kha transgene (also known as Tg2576, a plaque-forming, memory-impaired mouse modeling Alzheimer disease) that overexpress familial Alzheimer's disease (FAD) APP^{K670N/M671L} mutation (Hsiao 1996 Science 274:99-102).

Advantageously, the genetically engineered non-human animal of the present invention are predisposed to early onset of brain pathology phenotypes. This predisposition is advantageous in that it provides a novel model for the molecular analysis of brain pathologies and provides a model for discovery and evaluation of compounds for use in the treatment of brain pathologies. Methods of the present invention are discussed in detail below.

In one aspect, the genetically engineered animal of the present invention is predisposed to age-dependent accumulation of p-tau, NFTs, and neuronal death. Tau is a neuronal microtubule-associated protein found predominantly on axons. The function of tau is to promote tubulin polymerization and stabilize microtubules. Tau, in its hyperphosphorylated form, is a major component the neurofibrillary lesions in Alzheimer's disease brain. See, for example, J Neurosci 18:1743-1752, 1998 and Neuron 19:939-945, 1997. The accumulation of tau into neurofibrillary tangles is a pathological consequence of Alzheimer's disease, Parkinson disease, and other taupathies. To date, the animal of the present invention is the only model to demonstrate NFTs. This predisposition provides novel diagnostic and prognostic opportunities. Thus, a genetically engineered animal of the present invention provides, among other things, a novel model for studying disease progression, a model for screening for drugs for brain pathology, and a model for evaluating efficacy of drugs for brain pathology. Such methods are discussed in more detail below.

In another aspect, the genetically engineered animals of the present invention demonstrate inhibited angiogensis and/or inhibited tumorgenesis. In some embodiments, angiogenesis includes tumor angiogenesis. In some embodiments, tumorgenesis includes chemically induced tumorgenesis. In some embodiments, tumorgenesis includes tumor initiation and/or tumor growth. These predispositions provide novel diagnostic and prognostic opportunities. Thus, a genetically engineered animal of the present invention provides, among other things, novel models for identifying compounds with anti-angiogenic and/or anti-tumorgenic effect and methods for evaluating carcinogenicity of a candidate compound. Such methods are discussed in more detail below.

Also included in the present invention are genetically engineered cells that include an exogenous DNA as described herein. The exogenous DNA may be homozygous or heterozygous. Useful cells may be from, for example, mammals, fruit flies, nematodes, and fish. Preferably, the cell is from a mammal. More preferably the cell is from a human or a mouse. Optionally, the cell may be isolated from the genetically engineered non-human animal described above. These cell lines may be immortalized cell lines or they may be primary cells lines. The cells of the present invention may be kept in culture or in frozen stocks. Methods of isolating cells from tissues, immortalizing cells, and maintaining cells in culture are common knowledge within the art.

Genetically engineered animals and cells of the present invention may be produced using routine methods known in the art. As discussed, one type of genetically engineered non-human animal is a targeted animal. Targeted animals may be engineered by first producing the targeting cassette as described above. Briefly, the targeting cassette is typically engineered to contain a desired coding region for disruption, replacement, or duplication of a particular genomic. For instance, when making a genetically engineered animal by targeting, the introduced DNA may further include homology arms present at the 5′ and 3′ ends of the used to direct the location of homologous recombination and thereby control the site of genomic insertion. For instance, if targeting a heat shock protein genomic region, the targeting cassette may include homology arms that will direct the cassette to the heat shock protein genomic region, and may further result in a deletion of bases in the genomic region if desired.

The targeting cassette may be introduced into the genome of an embryonic stem (ES) cell. Typically, the targeting cassette is provided as a linear fragment and gDNA homology arms will undergo recombination with their matching sequences on one chromosome, carrying the targeting cassette with them. The gDNA between the regions of homology on the chromosome is thereby replaced by the targeting cassette and any other sequences flanked by the homology arms of the targeting construct. For a complete knockout, the target site is usually positioned to replace the TATA box, the start codon, and one or more of the initial exons.

Typically, the ES cells are screened for the presence and proper insertion of the targeting cassette. ES clones exhibiting desired resistance and/or sensitivity via selectable markers (as previously discussed) are selected and further examined for proper homologous recombination. For example, proper genomic insertion may be identified through molecular techniques that are known in the art such as southern blotting and sequencing.

ES cells harboring the properly integrated targeting cassette may be used to engineer a non-human animal. In a mouse, for example, the genetically-modified ES cells are then microinjected into host embryos at about the eight-cell blastocyst stage. These embryos are transferred to pseudopregnant host females. Preferably the ES cells and the host females are difference strains of mouse. More preferably, the strains differ in an easily distinguishable phenotype such as fur color. Host females then bear chimeric progeny. The chimeric progeny carrying the targeted mutation in their germ line are then bred to establish a line. If the newly established line has a disrupted or deleted gene, it is called a knock-out; if it has a new, replaced, or duplicated gene, it is called a knock-in. The presence of the exogenous DNA can be detected through methods for detecting DNA that are well known in the art such as PCR and Southern blotting. For targeting constructs engineered to express a coding sequence, expression of the coding sequence can be detected at either the transcriptional (RNA) or translational (protein) level. Methods of detecting RNA are well known in the art and include, for example, in situ hybridization, reverse-transcription PCR, and Northern blotting. Methods of detecting protein are well known in the art and include, for example, immunohistochemistry, immunocytochemistry, ELISAs, and immunoblotting (i.e. Western blotting).

Another type of genetically engineered animal is a transgenic animal. Transgenic animals may be engineered by first producing the segment of exogenous DNA. In a mouse, for example, the exogenous DNA is microinjected into the pronuclei of single-celled mouse embryos. These embryos are transferred to pseudopregnant host females and offspring are screened for the presence of the exogenous DNA. The presence of the exogenous DNA can be detected through methods for detecting DNA that are well known in the art such as PCR and Southern blotting. Expression of a coding region present on the exogenous DNA can be detected at either the transcriptional (RNA) or translational (protein) level. Methods of detecting RNA are well known in the art and include, for example, in situ hybridization, reverse-transcription PCR, and Northern blotting. Methods of detecting protein are well known in the art and include, for example, immunohistochemistry, immunocytochemistry, ELISAs, and immunoblotting (i.e. Western blotting).

A “knock-in” animal, as used herein, refers to a genetically modified animal in which a specific gene or part thereof is replaced by a foreign gene or DNA sequence. A “knock-out” animal, as used herein, refers to a genetically modified animal in which a gene is removed or rendered inoperative. As used herein the term “transgenic” refers to a genetically modified animal in which the endogenous genome is supplemented or modified by the random or site-directed integration of a foreign gene or sequence.

Animals with induced mutations may be produced by using a variety of mutagens. For example, one popular chemical mutagen, ethylnitrosourea (ENU), is used to induce point mutations. ENU mutagenesis involves exposing male animals to ENU and then mating the treated males to untreated females. The resultant progeny, many of which carry point mutations, are screened for phenotypes of interest. Other types to mutagens include, but are not limited to, ionizing radiation such as ultraviolet light, base analogs which can substitute for DNA bases and cause errors in replication, deaminating agents such as nitrous acid, intercalating agents such as ethidium bromide, alkylating agents such as bromouracil, and insertional mutagens such as transposons. These mutagenizing agents and methods of using them are common knowledge within the field.

Methods for genetically engineering cells are routine and are well known in the art. Cells may be engineered by first producing the desired exogenous DNA as described above through standard DNA recombination and cloning techniques. Optionally, the exogenous DNA includes homology arms present at the 5′ and 3′ ends of the DNA. The exogenous DNA may be introduced into a cell by a number of different methods. Non-limiting examples of methods for introducing the exogenous DNA into a cell include microinjection, transfection, transduction, and electroporation. Preferably, the exogenous DNA may insert into the genomic DNA of the cell. Insertion into the genomic DNA may be random or targeted.

Genetically engineered non-human animals of the present invention and cells of the present invention are useful for a variety of purposes. Genetically engineered animals having the characteristics of the reduced or eliminated function of a molecular chaperone, including, but not limited to, a hsp110 KO mouse and a Hsp70i KO mouse, are of special interest for a variety of applications. The genetically engineered non-human animals and cells, as described herein, provide novel model systems for analyzing the disease progression of brain pathology, angiogenesis and/or tumorgenesis. For example, such cells and genetically engineered non-human animals may be used as a model for studying disease progression; a model for screening for compounds useful in treating brain pathology; in methods for evaluating the efficacy of treatments for brain pathology; in methods for diagnosing brain pathology; in method for treating brain pathology; a model for screening for compounds useful in effecting angiogenesis and/or tumorgenesis; in methods for evaluating the efficacy of treatments for effecting angiogenesis and/or tumorgenesis; in methods for diagnosing or staging cancer; and in method for treating cancer and/or angiogenesis-related diseases.

The present invention also includes a method of screening for compounds that are useful in treating brain pathology. For example, a candidate compound is administered to an animal or isolated cell and indicators of brain pathology are evaluated. This may be compared to brain pathology in a second, control animal or cell, to which the candidate compound has not been administered. Reduced brain pathology in the experimental compared to the control indicates the candidate compound may be a compound useful for the treatment of a brain pathology. In a similar fashion, the present invention includes methods for evaluating the efficacy of treatments for brain pathology, wherein reduced brain pathology following administration of the compound indicates that the compound may be effective in treating brain pathology.

As previously discussed, brain pathology may be evaluated according to molecular phenotypes or cognitive phenotypes. Molecular phenotypes includes any of those described herein, including, for example, expression of Hsp70 and/or Hsp110, phosphorylation of tau, or aggregation of Aβ, p-tau, or a combination thereof. An agent which is useful for treating a neurologic disease may be identified by a decrease in such neuropathologic findings in the first non-human animal in comparison with the second non-human animal.

In one embodiment, brain pathology may be evaluated according to the expression of Hsp70 and/or the expression of Hsp110. For example, Hsp70 is known to co-localize with aggregates of Aβ. Therefore, detection of Hsp70 in the extracellular space may be indicative of brain pathology. Similarly, both Hsp70 and Hsp110 have been shown to co-localize with senile plaques. Therefore co-localization of Hsp70 and Aβ or co-localization of Hsp110 and Aβ may be indicative of brain pathology. Methods of detecting protein expression as well as localization are well known in the art and include, for example, immunohistochemistry, immunocytochemistry, ELISAs, and immunoblotting (i.e. Western blotting).

In another embodiment, brain pathology may be evaluated according to the appearance of p-tau in the brain. In a genetically engineered non-human animal of the present invention, for example, a hsp70i KO, p-tau can be detected in the brain by 10-14 weeks. Similarly, in a hsp110 KO mouse, p-tau can be detected in the brain by 4-6 weeks. Both mouse models demonstrate an age-dependent increase of p-tau. Therefore, if a hsp70i KO mouse is administered a candidate compound and the time required for p-tau to be detected in the brain is longer than 10-14 weeks, brain pathology is reduced and the candidate compound is a compound that is useful for the treatment of brain pathology. Likewise, if a hsp110 KO mouse is administered a candidate compound and the time required for p-tau to be detected in the brain is longer than 4-6 weeks, brain pathology is reduced and the candidate compound is a compound that is useful for the treatment of brain pathology.

In yet another embodiment, brain pathology may be evaluated according to the appearance of NFTs (aggregated p-tau) in the brain. In a genetically engineered non-human animal of the present invention, for example, in a hsp110 KO mouse, NFTs can be detected in the brain by 24-30 weeks. If a hsp110 KO mouse is administered a candidate compound and the time required for NFTs to be detected in the brain is longer than 24-30 weeks, brain pathology is reduced and the candidate compound is a compound that is useful for the treatment of brain pathology.

In still another embodiment, brain pathology may be evaluated according to the appearance of aggregated Aβ in the brain. A mouse harboring a mutated APP, for example the Tg2576 mouse (Hsiao 1996 Science 274:99-102), aggregated Aβ (or senile plaques) can be detected in the brain after 1 year. In a genetically engineered non-human animal of the present invention, for example, in a hsp110 KO mouse that further contains a mutated APP, aggregated Aβ can be detected in the brain by 7 months. For example, if a hsp110 KO mouse is administered a candidate compound and the time required for aggregated Aβ to be detected in the brain is longer than 7 months, brain pathology is reduced and the candidate compound is a compound that is useful for the treatment of brain pathology.

Cognitive evaluation may be assayed by any of a variety of methods. A number of assays commonly used with rodent models include, for example, an Alternating Lever Cyclic Ratio (ALCR) test, a delayed non-matching to place test, a morns water maze (commonly used to assess working memory in rats and mice), a delayed matching to sample test (an operant procedure for testing working memory), and a fixed-interval operant responding test (a sensitive procedure to assess non-specific cognitive effects, for example, when the type and anatomical location of the cognition being tested is unknown), a delayed conditioning procedure (representing a variety of operant or non-operant tests under which animals are exposed to stimuli paired with a reward or punishment and, after a delay, their ability to respond appropriately to the stimulus-reward combination is assessed), or a repeated acquisition procedure (an operant test, under which subjects are required to repeatedly learn a new stimulus sequence). A comparison of performance on a memory or learning tests of a first non-human animal contacted with the agent with that of a second non-human animal not contacted with the agent can be done.

The genetically engineered animals and cells of the present invention, having a reduced or eliminated function of a molecular chaperone, including, but not limited to, hsp110 knockout and Hsp70i knockout mice, can be used to screen compounds and treatments for oncogenic and antitumor activity.

The present invention include methods for evaluating carcinogenicity of a candidate compound. Such a method may include administering the candidate compound to an animal described herein, evaluating a tumor burden attained by the animal, and comparing the tumor burden attained by the animal to a tumor burden attained by a control animal, wherein a higher tumor burden present in the genetically engineered animal compared to the control animal indicates that the candidate compound is a carcinogen. Such a method may include administering the candidate compound to a cell described herein, evaluating the cell for transformation, and comparing the transformation of the cell to transformation of a control cell, wherein transformation in the cell compared to the control cell indicates the compound is a carcinogen.

The present invention also include methods for identifying a candidate anti-carcinogenic compound. Such a method may include administering a carcinogenic compound to an animal described herein, further administering the candidate anti-carcinogenic compound to the animal; evaluating a tumor burden attained by the animal; and comparing the tumor burden attained by the treated animal to a tumor burden attained by a control animal, wherein a lower tumor burden present in the treated animal compared to the control animal indicates the compound is an anti-carcinogenic compound. In some embodiments, the compound affects angiogenesis and/or tumorgenesis. Such a method may include administering a carcinogen to a cell described herein, administering the candidate anti-carcinogenic compound to the cell; evaluating the cell for transformation, and comparing the transformation of the cell to transformation of a control cell, wherein transformation in the cell compared to the control cell indicates the compound is a carcinogen.

The present invention includes methods of identifying compounds that alter the expression and/or function of a molecular chaperone, including, but not limited to Hsp70, the method including administering a candidate compound to a non-human transgenic animal as described herein, or an isolated cell as described herein and evaluating the expression and/or function of hsp70 in the animal or cell, wherein altered expression and/or function of hsp70 administration of the compound indicates that the compound is effective for altering the expression and/or function of heat shock protein 70 (hsp70). In some embodiments, altering the expression and/or function of heat shock protein 70 (hsp70) is reducing the expression and/or function of heat shock protein 70 (hsp70). In some embodiments, altering the expression and/or function of heat shock protein 70 (hsp70) is increasing the expression and/or function of heat shock protein 70 (hsp70). In some embodiments, the compound affects angiogenesis and/or tumorgenesis.

The present invention includes a method for identifying a compound with anti-angiogenic and/or anti-tumorgenic effect, the method including identifying a compound that reduces or eliminates the expression or function of the heat shock protein 70 (Hsp70) in a cell or animal described herein

The present invention includes agents identified by any of the methods described herein. Such agents may be used to effect the expression and/or function of a molecular chaperone, including, but not limited to, Hsp110 and/or Hsp70. In some aspects, such agents may effect the expression and/or function of a molecular chaperone protein by inhibiting the expression and/or function of a molecular chaperone protein, including, but not limited to, Hsp110 and/or Hsp70. In some aspects, such agents may effect the expression and/or function of a molecular chaperone protein by enhancing the expression and/or function of a molecular chaperone protein, including, but not limited to, Hsp110 and/or Hsp70. Such agents may used in methods of treating or preventing brain pathology, neurodegenerative diseases, angiogenesis-related conditions, and/or cancer.

Such agents may be administered to a subject to affect angiogenesis and/or tumorgenesis. A subject's response to a compound or treatment may be measured by any of a variety of parameters. Such parameters may be quantitative and/or qualitative, including, but are not limited to, tumor size, number of tumors, presence of tumor markers, differentiation of tumor tissue, extent of tumor cell death, and/or level of metastatic spread. Changes that occur to the parameters upon administration of the compound or treatment can include prevention, delayed onset, increase, or decrease in the parameter.

Such agents may be administered to a subject for the treatment or prevention of brain pathology in the subject. In some embodiments, the compound reduces the aggregation of Aβ and/or p-tau, inhibits phosphorylation of tau, reduces or eliminates p-tau, induces dephosphorylation of p-tau, and/or induces degradation of p-tau. A compound may reduce aggregation of Aβ, p-tau, or a combination thereof, may inhibit phosphorylation of tau, may induce dephosphorylation of p-tau, and/or may induce degradation of p-tau. Non-limiting example of brain pathologies include neurodegenerative diseases, tauopathies, and traumatic brain injury (TBI). As used herein, “taupathies” (also referred to herein as “tauopathies”) are a group of neurodegenerative disorders characterized by the presence of filamentous deposits and aberrant aggregates of hyperphosphorylated tau protein in neurons and glia. Neurodegenerative diseases include, but are not limited to, Alzheimer disease, amyotrophic lateral sclerosis, Parkinson's disease, polyglutamine diseases, Pick disease, dementia pugilistica, Down syndrome, argyrophilic grain dementia, diffuse neurofibrillary tangles with calcification, frontotemporal dementia and parkinsonism linked to chromosome 17, corticobasal degeneration, Niemann-Pick disease type C, progressive supranuclear palsy, progressive subcortical gliosis, tangle only dementia, tangle-predominant Alzheimer disease, non-guanamian motor neuron disease with neurofibrillary tangles, Gerstmann-Straussler-Scheinker disease, Hallervorden-Spatz disease, inclusion body myositis, Creutzfeld-Jakob disease, multiple system atrophy, Prion disease, prion protein cerebral amyloid angiopathy, subacute sclerosing panencephalitis, frontotemporal dementia, myotonic dystrophy, postencephalitic parkinsonism, and cortico-basal degeneration

The agents of the present invention can be used in both in vitro and in vivo diagnostic and therapeutic methods. Also included in the present invention are such in vitro and in vivo diagnostic and therapeutic methods. An agent may be administered to an individual in need thereof by any of a wide variety of means. For example, an agent may be delivered orally, subcutaneously, intramuscularly, intravenously, intrathecally, and/or intracranially. Delivery may be by local delivery or injection. Delivery may be by pump or extended release composition. An agent may be delivered prior to, during, and/or after delivery of another therapeutic agent. An agent may be delivered prior to during, and/or after the measurement of cognitive functioning. One or more agents may be administered. As used herein, “treating” a condition or a subject may include therapeutic, prophylactic, and/or diagnostic treatments. Treatment can be initiated before, during, and/or after the development of the condition to be treated. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

Suitable agents include any of a wide variety of biologically or chemically synthesized substances, including, but not limited to, polypeptides, oligopeptides, polysaccharides, nucleic acids, antibodies, antisense molecules, ribozymes, small chemical molecules, hormones, virus, amino acids, lipids, carbohydrates, drugs, prodrugs, and the like.

Also included in the present invention are compositions including one or more of the compounds described herein. A composition may include one or more accessory ingredients including, but not limited to, diluents, buffers, binders, disintegrants, surface active agents, thickeners, lubricants, preservatives (including antioxidants), solvents, diluents, antibacterial and antifungal agents, absorption delaying agents, carrier solutions, suspensions, colloids, and the like. A composition may further include additional therapeutic agents. The preparation and use of such compositions is well known in the art. A composition may be a pharmaceutically acceptable composition, meaning that the composition is not biologically or otherwise undesirable, i.e., the material may be administered to a subject without causing any undesirable biological effects or interacting in a deleterious manner with any of the other components of the pharmaceutical composition in which it is contained. A composition may be administered by any of a wide variety of means. For example, a compound may be delivered orally, subcutaneously, intramuscularly, intravenously, intrathecally, and/or intracranially. Delivery may be by local delivery or injection. Delivery may be by pump or extended release composition. An agent of the present invention may be isolated. By “isolated” is meant material that is substantially or essentially free from components that normally accompany it in its native state.

The present invention includes methods of evaluating brain pathology in a subject by detecting the co-localization of Hsp70 and Aβ and/or the co-localization of Hsp110 and Aβ in a sample obtained from the subject. The co-localization of Hsp70 and Aβ and/or the co-localization of Hsp110 and Aβ may be indicative of brain pathology. The detection of such co-localization may be indicative of a neurodegenerative disease. In some aspects, the detection of the neurodegenerative disorder or cognitive disorder may be presymptomatic. As used herein, a “sample” refers to a sample of tissue or fluid isolated from a subject, including but not limited to, for example, blood, plasma, serum, cerebrospinal fluid (CSF), or brain tissue and also samples of in vitro cell culture constituents including but not limited to conditioned media resulting from the growth of cells and tissues in culture medium, e.g., recombinant cells, and cell components.

The present invention includes methods of detecting a neurodegenerative disorder or cognitive disorder in a subject by detecting a polymorphic variant or a mutation in one or more hsp110 alleles in a nucleotide sample obtained from the subject. In some aspects, the detection of the neurodegenerative disorder or cognitive disorder may be presymptomatic.

The present invention includes methods of detecting a cancer or angiogenesis-related disorder in a subject by detecting a polymorphic variant or a mutation in one or more hsp70 alleles in a nucleotide sample obtained from the subject. In some aspects, the detection may be presymptomatic.

The detection of a polymorphic variant or a mutation may be by any of a variety of technologies, including, but not limited to hybridization, sequencing, and polymerase chain reaction (PCR)-based technologies. Such detection methods may include the transformation of matter. For example, the status of the sample after the detection step is altered from the status of the sample, as originally provided.

The detection methods of the present invention may include providing a report summarizing the results. Such a report may be provided, for example, in written or electronic formats. Polymorphic variants or a mutations may include, but are not limited to, allelic variants, mutations resulting in altered expression, missense mutations, silent mutations, deletion mutation, and intronic mutations.

The present invention and/or one or more portions thereof may be implemented in hardware or software, or a combination of both. For example, the functions described herein may be designed in conformance with the principles set forth herein and implemented as one or more integrated circuits using a suitable processing technology, e.g., CMOS. As another example, the present invention may be implemented using one or more computer programs executing on programmable computers, such as computers that include, for example, processing capabilities, data storage (e.g., volatile and nonvolatile memory and/or storage elements), input devices, and output devices. Program code and/or logic described herein is applied to input data to perform functionality described herein and generate desired output information. The output information may be applied as an input to one or more other devices and/or processes, in a known fashion. Any program used to implement the present invention may be provided in a high level procedural and/or object orientated programming language to communicate with a computer system. Further, programs may be implemented in assembly or machine language. In any case, the language may be a compiled or interpreted language. Any such computer programs may preferably be stored on a storage media or device (e.g., ROM or magnetic disk) readable by a general or special purpose program, computer, or a processor apparatus for configuring and operating the computer when the storage media or device is read by the computer to perform the procedures described herein. The system may also be considered to be implemented as a computer readable storage medium, configured with a computer program, where the storage medium so configured causes the computer to operate in a specific and predefined manner to perform functions described herein.

The present invention and/or one or more portions thereof include circuitry that may include a computer system operable to execute software to provide for the determination of a physiological state, e.g., heart failure. Although the circuitry may be implemented using software executable using a computer apparatus, other specialized hardware may also provide the functionality required to provide a user with information as to the physiological state of the individual. As such, the term circuitry as used herein includes specialized hardware in addition to or as an alternative to circuitry such as processors capable of executing various software processes. The computer system may be, for example, any fixed or mobile computer system, e.g., a personal computer or a minicomputer. The exact configuration of the computer system is not limiting and most any device capable of providing suitable computing capabilities may be used according to the present invention. Further, various peripheral devices, such as a computer display, a mouse, a keyboard, memory, a printer, etc., are contemplated to be used in combination with a processing apparatus in the computer system. In view of the above, it will be readily apparent that the functionality as described herein may be implemented in any manner as would be known to one skilled in the art.

The present invention is illustrated by the following examples. It is to be understood that the particular examples, materials, amounts, and procedures are to be interpreted broadly in accordance with the scope and spirit of the invention as set forth herein. All headings are for the convenience of the reader and should not be used to limit the meaning of the text that follows the heading, unless so specified.

### EXAMPLES

### Example 1

Loss of Heat Shock Protein (Hsp) 110 Leads to Tau Hyperphosphorylation and Early Accumulation of Insoluble Amyloid-β Peptide in Mouse Model of Alzheimer's Disease

Accumulation of tau into neurofibrillary tangles is a pathological consequence of Alzheimer's disease, Parkinson's disease, and other tauopathies. Failures of the quality control mechanisms by the heat shock proteins (Hsps) have been positively correlated with the appearance of such neurodegenerative diseases. However, in vivo genetic evidence for the role of Hsps in neurodegeneration remains elusive. Hsp110 is a nucleotide exchange factor for the Hsp70 family and has been named a “holdase” because direct substrate binding to Hsp110 may facilitate substrate folding. To provide genetic evidence for a potential role for Hsp110 in neurodegeneration, we have generated hsp110^{-/-} mice. Our results show that absence of the Hsp110 gene in mice leads to an age-dependent accumulation of hyperphosphorylated tau. Towards determining the underlying mechanisms of neurodegeneration in hsp110^{-/-} mice we have found that Hsp110 interacts with tau, and the peptidyl-prolyl isomerase (PPIase) Pin1, and that hsp110^{-/-} brain extracts exhibit lower PPIase activity. Mice deficient in the Hsp110 partner protein, hsp70 also accumulate phospho-tau indicating a critical role for Hsp110-Hsp70 complex in maintaining tau in its unphosphorylated form. In addition, Hsp110 interacts with amyloid precursor protein (APP), and crossing Tg2576⁺ mice overexpressing mutant form of APP with hsp110^{-/-} mice leads to the appearance of insoluble Amyloid β42 (Aβ42) at a younger age, suggesting an essential role for this molecular chaperone in tau phosphorylation, APP processing, and Aβ generation. Thus, this example provides in vivo evidence that the Hsp110-Hsp70 plays a critical function in neurodegeneration such as Alzheimer's disease and other tauopathies.

Misfolded proteins are either degraded through the ubiquitin proteasome system (UPS) or are folded at least in part by the heat shock proteins (Hsps) (Barral et al., 2004 Semin Cell Dev Biol 15:17-29; Bukau et al., 2006 Cell 125:443-451). Eukaryotic cells possess a class of Hsps related to the Hsp70 family. This Hsp100 family of proteins contains Hspa41 (Apg1 or OSP94), Hsp94 (Apg2), and Hsp110 (Tsapara et al., 2006 Mol Biol Cell 17:1322-1330; Yamagishi et al., 2006 Exp Cell Res 312:3215-3223; Gashegu et al., 2007 J Anat 210:532-541; Hrizo et al., 2007 J Biol Chem 282:32665-32675; Saito et al., 2007 Exp Cell Res 313:3707-3717; Yamashita et al., 2007 J Neurochem 102:1497-1505; Andreasson et al., 2008 J Biol Chem 283:8877-8884). They were initially considered to be “holdases” that keep denatured proteins in solution, and no client proteins have been described for them (Easton et al., 2000 Cell Stress Chaperones 5:276-290; Dragovic et al., 2006 EMBO J. 25:2519-2528; Polier et al., 2008 Cell 133:1068-1079; Schuermann et al., 2008 Mol Cell 31:232-243). Hsp110 interacts with Hsp70 and increases its ATPase activity (Easton et al., 2000 Cell Stress Chaperones 5:276-290; Polier et al., 2008 Cell 133:1068-1079; Schuermann et al., 2008 Mol Cell 31:232-243). The main function of Hsp110 appears to be a nucleotide exchange factor (NEF) for Hsp70 (Dragovic et al., 2006 EMBO J. 25:2519-2528; Shaner et al., 2006 Biochemistry 45:15075-15084). In general, Hsp110 is known to induce suppression of aggregation, protein refolding, and protects proteins from the damaging effects of various stresses; however, its physiological function in mammalian cells remains unknown (Easton et al., 2000 Cell Stress Chaperones 5:276-290; Saito et al., 2007 Exp Cell Res 313:3707-3717).

The common features observed in Alzheimer's disease (AD) and Parkinson's disease (PD) are the accumulation of aggregated proteins (Ballatore et al., 2007 Nat Rev Neurosci 8:663-672). Diseases such as AD and other tauopathies are defined by the expression of neurofibrillary tangles (NFTs) deposited mainly in neurons. The NFTs are aggregates of the hyperphosphorylated tau (Williams, 2006 Intern Med J 36:652-660; Ballatore et al., 2007 Nat Rev Neurosci 8:663-672). Normal tau increases microtubule stability, but can be hyperphosphorylated under disease conditions, and released from microtubules. The molecular mechanisms involved in the formation of NFTs are not completely understood. However, accumulation of phospho-tau and NFTs cause neurodegeneration (Ballatore et al., 2007 Nat Rev Neurosci 8:663-672).

Hsp70/Hsc70 has been shown to preferentially bind to a hyperphosphorylated form of tau in the diseased human brain (Muchowski and Wacker, 2005 Nat Rev Neurosci 6:11-22). Cross-talk between the ubiquitin proteasome system (UPS) and molecular chaperones might also be critical in regulating the deposition and toxicity of tau (Caims et al., 2004 J Pathol 204:438-449; Esser et al., 2004 BBA 1695:171-188). These results suggest that the activity of Hsp70 and Hsp90 preserve the native structure and function of tau protein. Hsp70 and the C-terminal Hsp70 interacting protein (Chip) have been shown to regulate tau ubiquitination and degradation (Petrucelli et al., 2004 Hum Mol Genet. 13:703-714; Shimura et al., 2004 J Biol Chem 279:4869-4876; Dickey et al., 2006 J Neurosci 26:6985-6996; Dickey et al., 2007 J Clin Invest 117:648-658; Goryunov and Liem, 2007 J Clin Invest 17:590-592). Interestingly, Chip and amyloid β precursor protein (βAPP) interact, and Chip and Hsp70/90 expression have been shown to lower the cellular levels of Aβ and reduce Aβ toxicity in vitro (Kumar et al., 2007 Hum Mol Genet. 16:848-864). Accumulation of intracellular Aβ42 occurs early during AD pathology and causes neuronal loss and clinical dementia (Kumar et al., 2007 Hum Mol Genet. 16:848-864).

In these studies, we examined the role of Hsp110 in central nervous system (CNS) homeostasis in vivo. We have found that hsp110^{-/-} mice exhibit an age-dependent accumulation of phospho-tau that is associated with pathological features such as cellular apoptosis and appearance of NFTs. We also show that lack of Hsp110 leads to accelerated pathology as evidenced by the early appearance of senile plaques containing Aβ42 (a major toxic species (Mattson, 2004 Nature 430:631-639)) in an AD transgenic mouse model. Our studies suggest a critical role for Hsp110 in tau phosphorylation and AD pathology.

### Methods and Materials

Generation of hsp110-deficient mice. To generate the hsp110 targeting vector, a 129/SvJ mouse genomic DNA phage library (Lambda fixII vector, Stratagene, La Jolla, Calif.) was used to identify clones containing the hsp110 gene using a mouse hsp110 cDNA as a probe. This 514-base pair (bp) probe spanned exons 1 to 5 and was amplified by PCR using forward 5′-GGG GGA TCC ATG TCG GTG GTT GGG CTA GAC G-3′ (SEQ ID NO:1) and reverse 5′-GCA AGC AGT TCA AGC CCA CAA TCT-3′ (SEQ ID NO:2) primers. Targeting vector construction was based on a lacZ-neo-tk (pN-Z-tk2) template plasmid vector containing a c-galactosidase (lacZ) gene fragment with the bovine growth hormone poly (A) signal (lacZ-poly(A)), a neomycin resistance gene driven by the thymidine kinase (tk) promoter with the simian virus 40 poly(A) signal (tk/neo-poly(A)), and flanking tk gene cassettes (Huang, 2001. Mol. Cell. Bio. 21:8575-8591). The tk/neo-poly(A) fragment was flanked by Cre recombinase recognition (loxP) sequences to allow removal of the selectable marker gene (Neo) from the targeted locus by intercrossing the mutant mice with transgenic mice expressing the cre recombinase gene (B6.C-Tg(CMV-cre) cgn/J (Jackson Laboratory, Bar Harbor, Me.)). In the targeting construct, Hsp110 exon 1 at ATG and 910 by of intron 1 were deleted and replaced in-frame with a 3.5 kilobase lacZ-neomycin cassette using a 2.5 kilobase insert of hsp110 at the proximal end and a 5 kilobase insert of hsp110 gene at the distal end. The 2.5 kb proximal region was amplified using forward 5′-ACG CGT CGA CGA TCC TTC TTA AAA ATC TAC-3′ (SEQ NO:3) and reverse primers 5′-GTA AAG CTT GGC TGG CCC GGT CCG CCT C-3′ (SEQ ID NO:4) and contained SalI and HindIII restriction enzyme sites. The 5 kb distal end contained restriction enzymes NotI and XhoI. NotI site was generated by PCR, and XhoI site is located downstream of exon 7 in the hsp110 gene.

The DNA fragments were ligated into the pN-Z-tk2. The final hsp110 targeting vector was sequenced, mapped, and the vector was linearized by SalI restriction enzyme and electroporated into C57BL/6 ES cells and G418 resistant, ganciclovir-sensitive, clones were selected. From the 135 isolated ES clones that were analyzed by Southern blotting, 20 clones contained correctly targeted allele using a probe external to the targeting vector. Two positive ES clones were injected into C57BL/6J blastocysts, and the resulting chimeric male mice were crossed with C57BL/6J females to generate germ-line transmission and an hsp110 heterozygous mouse line. The hsp110 mice were born with the expected Mendelian frequency and were phenotypically normal and fertile for a period of at least one year.

Mouse lines. The hsp110^{-/-} mice were in C57BL/6 genetic background. Tg2576⁺ transgenic mice were purchased from Taconic (Hudson, N.Y.) and were in C57BL/6J SJL genetic background. The Tg2576⁺ transgenic mice overexpress a 695 amino acid splice form (Swedish mutation K670N M671I) of the human APPβ (Hsiao et al., 1996 Science 274:99-102). Generation of hsp70i^{-/-} mice (C57BL/6 genetic background) will be reported elsewhere.

Southern blot analysis and genotyping. Genomic DNA was digested with BamHI and fractionated on 0.8% agarose gel and transferred to a nylon membrane (Hybound-N+, Amersham Biosciences). The probe used for Southern blot analysis was a 730 by fragment of Hsp110 cDNA that was PCR amplified from genomic DNA using the following primers: 5′-GGG GGA ATT CCT AGG ATG GGC AAA G-3′ (SEQ ID NO:5) and 5′-GGC GGA ATT CGA TGC CCT TTC AAG AA-3′ (SEQ ID NO:6). The probe was labeled by random priming with α³²P-CTP using NEBlot Kit (New England Biolabs, Inc., MA). Wild-type and mutant Hsp110 genomic DNA digested with BamHI generated a 15.6 kb and 13 kb fragments, respectively. For routine genotyping of mice, DNA extracted from tail DNA was used in multiplex PCR analysis to verify a wild-type 242 bp, and a mutant 405 by fragments, using primers 1: 5′-ACATAAGGCTGAGCGATTGG-3′ (SEQ ID NO:7); 2: 5′-ATGTAGCAGCTCTGT-GAGCCTAC-3′ (SEQ ID NO:8); and 3: 5′-CAGGAAGATCGCACTCCAG-3′ (SEQ ID NO:9). Primers 1 and 2 were located in exon 1, and Primer 3 was located in the LacZ cDNA (Zhang, 2002 J Cell Biol 86: 376-393; Min, 2004 Genesis 40:205-217).

Primary neuronal cell culture. Neuronal cultures were prepared from cortices extracted from E18 days-old embryos as previously reported (Homma et al., 2007 J Neurosci 27:7974-7986).

Sarkosyl-soluble and insoluble fractions of brain tissue. To prepare Sarkosyl soluble and insoluble fractions, the method of Dickey et al. (2006 J Neurosci 26:6985-6996) was used. Brain tissue was homogenized in 50 mM Tris pH 8.0, 5 mM KCl, 274 mM NaCl buffer, and a cocktail of proteases and phosphatase inhibitor (10 mM sodium fluoride and 2 mM sodium vanadate). The homogenate was subjected to centrifugation at 60,000 rpm, for 15 min, and the supernatant was the soluble 51 fraction. The pellet was further homogenized in 10 mM Tris HCl, pH 7.4, 0.8 M NaCl, 10% sucrose, 1 mM EGTA buffer and re-ultracentrifuged. The supernatant from the second centrifugation step was mixed with 1% Sarkosyl and incubated for 1 hour at 37° C. The second extract was subjected to ultracentrifugation, for 30 min, and the supernatant was the Sarkosyl-soluble (S2 fraction). The pellet was the Sarkosyl-insoluble (P3 fraction) and was resuspended in Tris-EDTA.

Measurements of α and β secretase activities and Aβ production. The α (Cat# FP001) and β (Cat #FP002) secretase activities were performed using kits purchased from R&D (Minneapolis, Minn.) according to manufacturer's instructions. Mouse Aβ40 (Cat #KMB3481), Mouse Aβ42 (Cat #KMB3441), human Aβ40 (Cat #KRB3481) and human Aβ42 (Cat #KHB3441) were detected using Immunoassays (calorimetric) kits purchased from Invitrogen (Camarillo, Calif.) and performed according to manufacturer's instructions. Preparation of soluble and insoluble fractions of brain extracts were as suggested by the manufacturers.

Peptidyl-propyl cis-trans isomerase activity. PPIase activity was determined as previously described with minor modifications (Sultana et al., 2006 Neurobiol Aging 27:918-925). Briefly, 200 μl of assay mixture contained 4 μl of fresh brain cell lysate (2 mg/ml), 1 μl of α-Chymotrypsin (60 mg/ml; Sigma) and 193 μl of HEPES buffer (32 mM, pH 7.8). The reaction started by adding 2 μl of substrate (25 mg/ml N-succinyl-Ala-Ala-Pro-Phe p-nitroanilide) (Sigma). For control groups, brain cell lysates were replaced with HEPES buffer. The reaction was followed at 395 nM for 0-10 min.

Purified proteins. The purified Hsp110 (human) was a generous gift from Dr. J. Subjeck (Roswell Park, N.Y.). The purified tau 441 was purchased from rPeptide (Bogart, Ga.).

Immunoprecipitation and immunoblotting. Brain tissue was homogenized in RIPA buffer containing 150 mM NaCl, 1% NP-40, 0.5% deoxycholate, 0.1% SDS, 50 mM Tris, pH7.5, 1× cocktail of protease inhibitor, 10 mM phenylmethyl sulphonyl fluoride (PMSF), 1% sodium pyrophosphate, 10 mM sodium fluoride and 2 mM sodium vanadate. Samples were centrifuged at 10,000 g for 20 min. at 4° C. Immunoprecipitation and immunoblotting assays were performed as previously described (Hu, 2006 Mol Cell Biol 8:3282-3294). Primary antibodies were as follows: Hsp90, Hsc70, Hsp60, and Hsp40 (Stressgen); Pin1 (Santa Cruz); Hsp110 (Santa Cruz sc-1804; sc-6241); Chip (Cell Signaling); NeuN (Chemicon); GFAP (Dako); βAPP (Sigma, Saint Louis, Mo.) (Cat#NAB228, this antibody recognizes human β-amyloid peptide, full-length APP, soluble APPβ′ and APPα, C99 cleavage form and Aβ (1-40/42)); sAPPβsw (Cat#10321, this antibody recognizes human sAPPβsw) (Invitrogen, Camarillo, Calif.). Tau and phospho-tau antibodies were a gift from Dr. P. Davies (Albert Einstein School of Medicine, N.Y.).

Histology and immunohistochemistry. Brains were fixed in 4% paraformaldehyde and embedded in paraffin. For immunostaining, 7 μm tissue sections were deparaffinized in xylene and rehydrated in series of alcohol/water mixtures. Antigen retrieval was performed by placing slides in 10 mM sodium citrate, pH 6.0 and steam for 30 min. After rinsing in Phosphate Buffered Saline (PBS), tissue sections were blocked in 3% BSA in PBS for 2 hours at 4° C. Tissue sections were incubated in primary antibody diluted in 3% BSA in PBST for 16 hours at 4° C. Antibody/antigen were detected with Cy3 fluorescent-conjugated anti-mouse (or rabbit) IgG secondary antibody. Nuclei were stained with DAPI (Homma et al., 2007 J Neurosci 27:7974-7986). For β-galactosidase staining of tissue sections, cryosections were stained as described previously (Huang et al., 2007 Genesis 45:487-501).

Behavioral studies. Behavioral studies were performed with wild-type and hsp110^{-/-} male mice at 12 months of age in the Small Animal Behavioral Core at the Medical College of Georgia using standard protocols (Ladurelle et al., 2000 Brain Res 858:371-379; Ma et al., 2007 Neuroscience 147:1059-1065). To test for motor function, the ability of mice to balance for 45 seconds on a 1.2 cm diameter bar fixed at a height of 40 cm was determined (Sereda et al., 1996 Neuron 1:1049-1060).

Statistical consideration. All experiments were performed at least three times. Number of mice used for each time point and/or genotype were n=3-8. Multiple sections of tissues (n=5-10) from each mouse were analyzed for each histological or immunohistological analyses. For statistical analyses data were expressed as mean+/−Standard Error of the Mean (SEM) and unpaired two-tailed Student's t-test. In some analyses, one-way analysis of variance (ANOVA) was used to compare groups as indicated in the Figure legend. Differences between groups were considered significant at p<0.05.

### Results

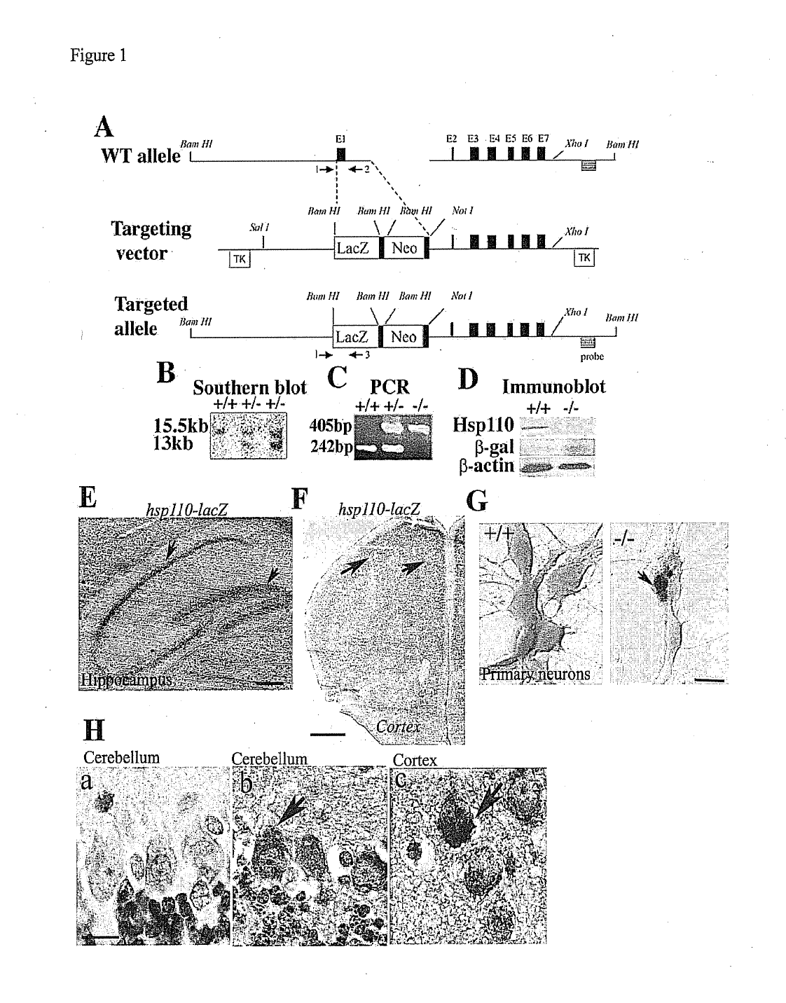

Hsp110 is expressed in the CNS. To investigate the physiological role of Hsp110 in the CNS, we generated mice with targeted disruption of the hsp110 gene and replacing the gene with β-galactosidase (FIGS. 1A-D). Previous reports indicate that Hsp110 is expressed in neurons in the cerebral cortex, hippocampus, thalamus, hypothalamus, and in the Purkinje cells of the cerebellum (Easton et al., 2000 Cell Stress Chaperones 5:276-290; Hylander et al., 2000 Brain Research 869:49-55). To confirm and extend these observations, we performed lacZ staining of different regions of the CNS using hsp110^{-/-}-lacZ reporter mice. The LacZ staining of hippocampus and the cortical regions are presented in FIGS. 1E and 1F. The results indicate that the hsp110-lacZ is expressed in both of these regions of the brain. We also stained cultured neurons for the presence of lacZ (FIG. 1G). The data indicate that hsp110-lacZ is expressed constitutively in the cortical neurons during embryogenesis. Using antibody to Hsp110, immunohistochemical staining shows Hsp110 expression in the Purkinje cells in the cerebellum and cortical neurons (FIG. 1H (b-c)). FIG. 1H, panel (a) represents negative control.

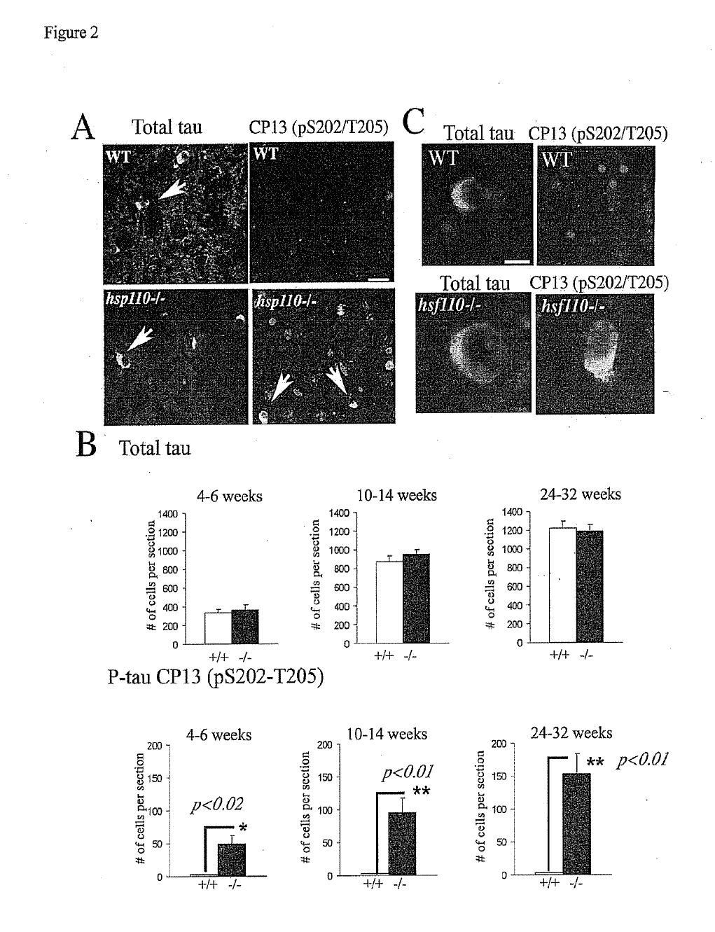

Hsp110^{-/-} mice exhibit an age-dependent accumulation of hyperphosphorylated tau (p-tau). Several molecular chaperones, such as Hsp70, Hsp90, and Hsp27, have been shown to interact with various microtubule components including tau (Liang and MacRae, 1997 J Cell Sci 110:1431-1440). Since Hsp110 interacts with Hsp70, we examined the expression of tau, a microtubule-associated protein, and phospho-tau (p-tau) in the presence, or absence of the Hsp110 gene. The results show that total tau (DA9 antibody) expresses in neurons in both wild-type and hsp110^{-/-} brain tissue (FIG. 2A). Surprisingly, a significant number of neurons also stained positively for p-tau (CP13) (S202/T205) in hsp110^{-/-} mice (FIG. 2A). Quantitation of the cells expressing total tau, or p-tau at S202/T205 in the hippocampal region of wild-type and hsp110^{-/-} mice indicated a significant increase of p-tau in hsp110^{-/-} brain tissue increasing with age (FIG. 2B). The positive immunoreactivity for the p-tau was only apparent in hsp110^{-/-} brain tissue, and not in wild-type at all the age groups. FIG. 2C shows the p-tau accumulation in hsp110^{-/-} brain tissue sections at higher magnification. In addition to the hippocampal region, CP13 immunoreactivity was also evident in the corpus callosum and medulla.

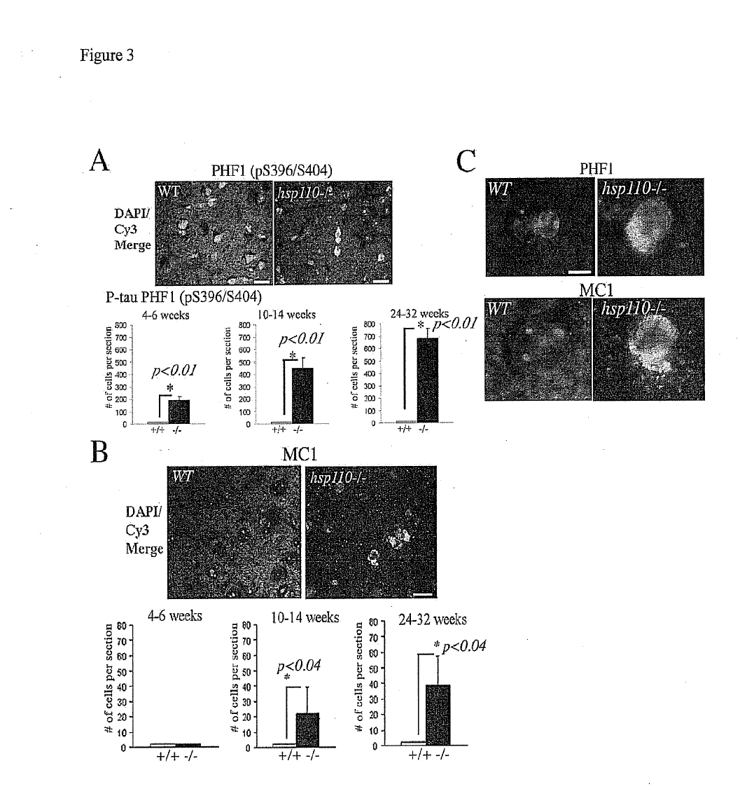

Immunostaining of the sections of the hippocampal region from hsp110^{-/-} mice also exhibited positive immunoreactivity to PHF1 antibody, which recognizes p-tau at p5396/S404 (FIG. 3A). Similarly, quantitation of the positively stained cells in hippocampal region of hsp110^{-/-} mice at 4-6, 10-14, and 24-32 weeks of age indicated a significant (p<0.01) increase in immunoreactivity in hsp110^{-/-} brain tissue compared to wild type (FIG. 3A). In addition to hippocampus, PHF1 immunoreactivity was also observed in the corpus callosum and medulla of hsp110^{-/-} mice.

Previous studies indicate that the ubiquitin ligase Chip (C-terminal Hsp70-interacting protein) participates in the clearance of p-tau. Indeed, chip^{-/-} mice exhibit immunoreactivity to CP13 (S202/T205) and 12E8 (S262/S356) p-tau antibodies. However, chip^{-/-} mice do not exhibit any immunoreactivity to MC1 which recognizes the Alzheimer's-type conformational tau epitope (Dickey et al., 2006 J Neurosci 26:6985-6996). In contrast, previous data indicate that pin1^{-/-} mice exhibit positive immunoreactivity to many phospho-specific tau antibodies including MC1 (Liou et al., 2003 Nature 424:556-561). Therefore, to examine whether hsp110^{-/-} neurons show MC1 immunoreactivity, we immunostained tissue sections from olfactory region (forebrain) of hsp110^{-/-} mice (FIG. 3B). The data indicate that hsp110^{-/-} neurons exhibit positive immunoreactivity to the conformational specific tau antibody. Quantitation of the data indicates a significant age-dependent increase in the number of immunoreactive p-tau in hsp110^{-/-} brain compared to wild-type mice (p<0.04) (FIG. 3B). The data presented in FIG. 3C shows neurons expressing p-tau detected by PHF1 or MC1 antibodies at a higher magnification.

These results indicate that hsp110^{-/-} brain tissue exhibit positive immunoreactivity to p-tau specific antibodies and this increases with age.

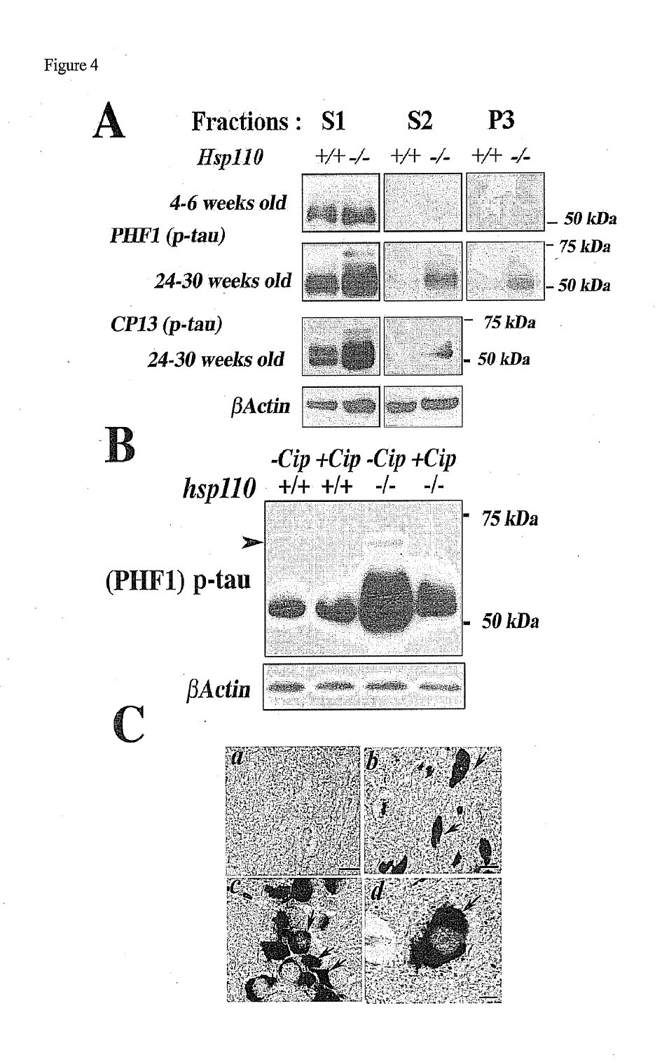

Presence of p-tau and neurofibrillary tangles (NFTs) in hsp110^{-/-} brain of aging mice. To confirm the observation made with immunohistochemical staining, we performed biochemical analyses and detected p-tau in Sarkosyl-soluble and insoluble fractions (Dickey et al., 2006 J Neurosci 26:6985-6996) prepared from hsp110^{-/-} and wild-type aging brains. Results indicate that the soluble (S1) and Sarkosyl soluble (S2) fractions of hsp110^{-/-} brain extracts exhibit the presence of p-tau using phosphospecific antibodies, PHF1 or CP13 at 24-30 weeks of age, but not in mice at 4-6 weeks of age (data for PHF1 is presented) (FIG. 4A). At 24-30 weeks of age, p-tau could be detected in the Sarcosyl-insoluble pellet fraction (P3) using PHF1 antibody in hsp110^{-/-} brain extracts (FIG. 4A). Presence of tau in the soluble brain extracts of hsp110^{-/-} mice was confirmed by immunoblotting using PHF1 antibody following treatment of cell extracts with calf intestinal alkaline phosphatase (CIP) (FIG. 4B). Treatment of hsp110^{-/-} brain tissue extracts with CIP generated a 55 kDa band, comparable to the unphosphorylated tau.

One of the hallmarks of neurodegeneration that is also evident in brains expressing p-tau is the presence of NFTs (Ballatore et al., 2007 Nat Rev Neurosci 8:663-672). NFTs were detectable in both cerebral cortex (FIG. 4C (b and d)), and hippocampal (FIG. 4C(c)) regions of the brain of 24-32 weeks-old hsp110¹, but not wild-type (FIG. 4C(a)) mice using Bielschowsky staining.

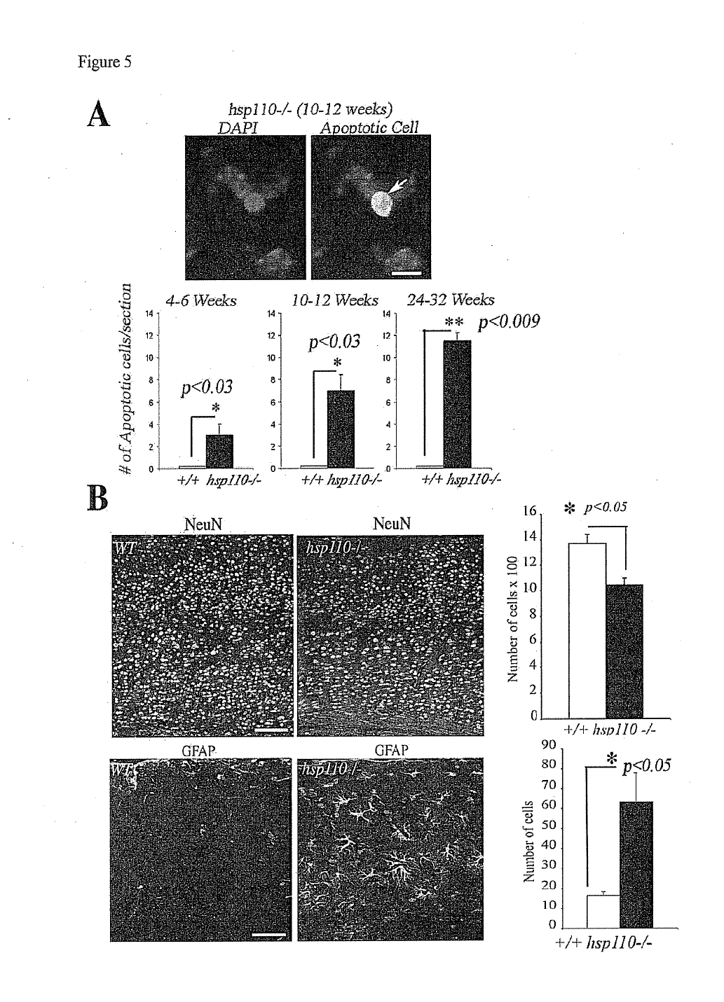

Accumulation of p-tau has been shown to induce apoptosis of neurons leading to neurodegeneration (Kosik and Shimura, 2005 Biochim Biophys Acta 1739:298-310; Ballatore et al., 2007 Nat Rev Neurosci 8:663-672). To determine whether the number of apoptotic cells increases with age in hsp110^{-/-} brains, we quantitated the number of apoptotic cells in the hippocampus region of the brain tissue sections following TUNEL staining. As the data in FIG. 5A indicate, the number of TUNEL-positive cells is significantly higher in hsp110^{-/-} brain tissue than in comparable areas of the wild-type brain. The increase in apoptotic cells in hsp110^{-/-} brain occurs in an age-dependent manner (FIG. 5A, lower panels).

A significant reduction in the neuronal specific marker, NeuN positive cells in the cortical region of hsp110^{-/-} brain was apparent compared to wild-type mice (FIG. 5B, upper panels). An increase in reactive astrocytes (astrogliosis) has been shown to be prominent in areas with high levels of p-tau immunostaining and neuronal cell death (Masliah and Rockenstein, 2000 J Neural Transm Suppl 59:175-183). As the data in FIG. 5B (lower panels) indicate, the cortical region of the brain of hsp110^{-/-} mice exhibits a significant increase in the glial fibrillary acidic protein (GFAP) immunopositive cells. This was not detected in comparable areas of the brain in wild-type mice (FIG. 5B, lower left panel).

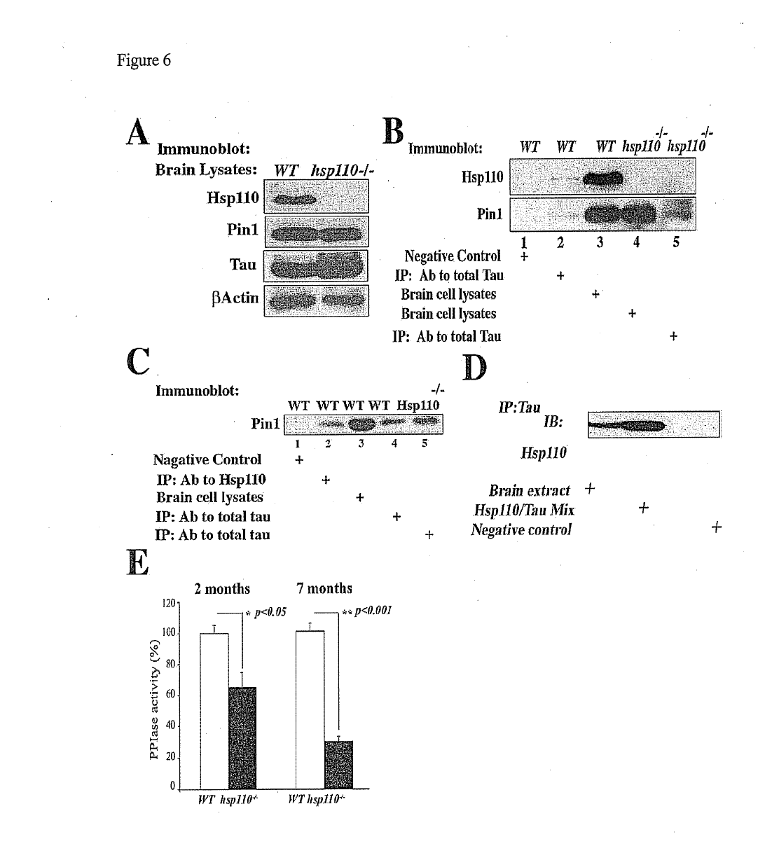

Hsp110 is present in tau and Pin1 immunocomplexes, and hsp110^{-/-} brain extracts exhibit reduced PPIase activity. Tau has previously been shown to interact with Pin1 isomerase (Lu et al., 1999 Nature 399:784-788). In addition, Pin1 appears to facilitate cis-trans isomerization of tau at threonine 231 (Lu et al., 1999 Nature 399:784-788; Smet et al., 2004 Biochemistry 43:2032-2040), and pin1^{-/-} mice exhibit p-tau, NFTs, and neurodegeneration (Liou et al., 2003 Nature 424:556-561). Initially, to ensure brain extracts of hsp110^{-/-} mice expressed Pin1 similar to the wild-type mice, immunoblotting experiments were performed. The results indicate that high level of Pin1 is expressed in brain extracts of both hsp110^{-/-} and wild-type mice (FIG. 6A). Expression levels of Hsp110 and tau are presented as controls. Next, to determine whether Hsp110 interacts with Pin1 and tau, brain extracts from wild-type or hsp110^{-/-} mice were subjected to immunoprecipitation analyses using antibody to total tau, and immunoblotting was performed to detect Hsp110 or Pin 1 (FIG. 6B). The results show that antibody to total tau can immunoprecipitate Hsp110 or Pin 1 from wild-type brain extracts (FIG. 6B, lane 2), and not from hsp110^{-/-} brain extracts as expected (FIG. 6B, lane 5, upper panel). We also tested whether total tau can pull-down Pin1 as has previously been shown by others (Lu et al., 1999 Nature 399:784-788). We found that antibody to total tau immunoprecipitated Pin1 from both wild-type and hsp110^{-/-} brain extracts (FIG. 6B, lanes 2 and 5, lower panel). In a comparable experiment, brain extracts from wild-type or hsp110^{-/-} mice were subjected to immunoprecipitation using antibody to Hsp110 or to total tau (FIG. 6C). Immunoblotting was performed to detect Pin1. The results show that both antibodies to Hsp110 or to total tau could immunoprecipitate Pin1 from wild-type brain extracts (FIG. 6C, lanes 2 and 4). Again antibody to total tau could pull-down Pin1 from hsp110^{-/-} brain extracts (FIG. 6C, lane 5). The interaction of tau with Pin1 likely is direct (Lu et al., 1999 Nature 399:784-788).

As the data in FIG. 6 indicates, Hsp110 is present in tau immunocomplexes; however, it is not clear whether the interaction is direct or indirect. In order to determine whether Hsp110 interacts with tau directly, we incubated purified wild-type tau441 and Hsp110 and subjected them to immunoprecipitation analyses. Immunoblotting of the pulled-down materials suggest that Hsp110 and tau interact directly (FIG. 6D). These results indicate that Hsp110 interacts with tau and Pin 1 and is directly involved in regulation of tau phosphorylation state.

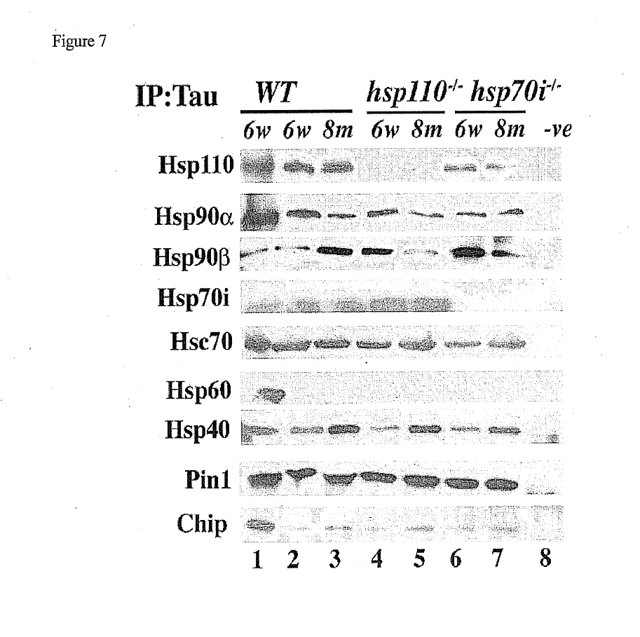

Pin1 activity is critical for proper dephosphorylation of phosphorylated tau (Liou et al., 2003 Nature 424:556-561). Since we found that Pin1 interacts with both tau and Hsp110 in brain extracts, we compared the activity of PPIase in hsp110^{-/-} brain extracts to that of wild type (Sultana et al., 2006 Neurobiol Aging 27:918-925). As the data in FIG. 6E indicates, PPIase activity is significantly lower in brain extracts of hsp110^{-/-} mice at 2 month (p<0.05) or at 7 month of age (p<0.001) compared to wild-type mice. Immunoblotting analyses did not show a significant reduction in the level of Pin1 in the soluble fraction of 2 or 7-month-old brain extracts from wild-type or hsp110 mice. Previous reports indicate that Hsp90 and Hsp70 as well as their co-chaperones are in complexes with p-tau (Dickey et al., 2007 J Clin Invest 117:648-658). To determine whether Hsp110, Pin1 together with other Hsps are in tau immunocomplexes, using young and old wild-type, hsp110^{-/-}, and hsp70i^{-/-} brain extracts, we immunoprecipitated total tau and examined the presence of Hsp90, Hsp70, Hsp110, and Pin1 as well as other chaperones in tau complexes (FIG. 7). The results show that majority of these proteins are found in tau immunocomplexes in wild-type, hsp110^{-/-}, and hsp70i^{-/-} brain extracts. We also used p-tau (PHF1) antibody, to immunoprecipitate Hsp110. Results show that Hsp110 was present in p-tau immunocomplexes pulled-down from wild-type, or hsp70i^{-/-} brain extracts. These results indicate that Hsp110 is associated with tau (or p-tau) together with other Hsps in the wild-type or hsp70i^{-/-} brain extracts.

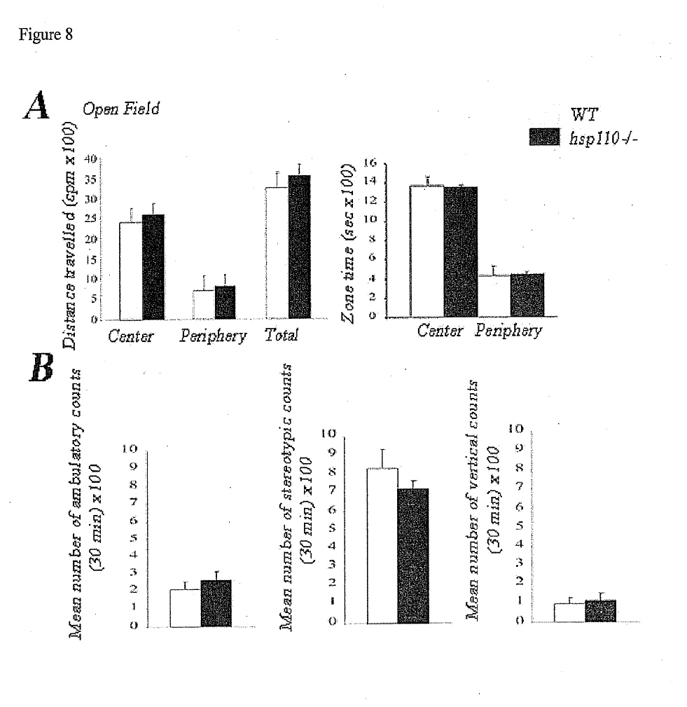

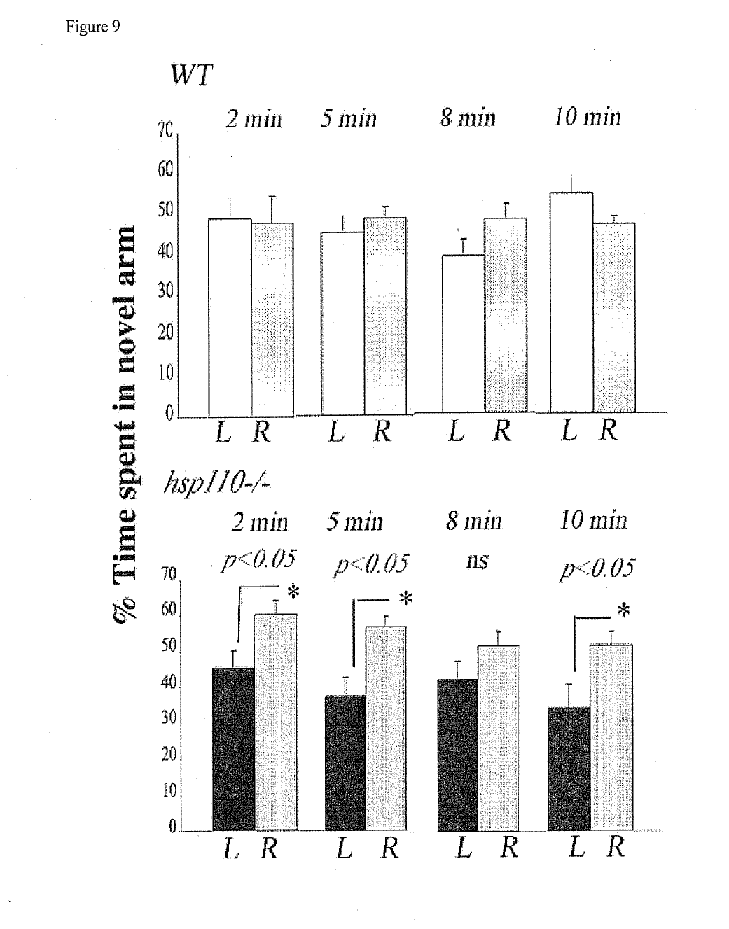

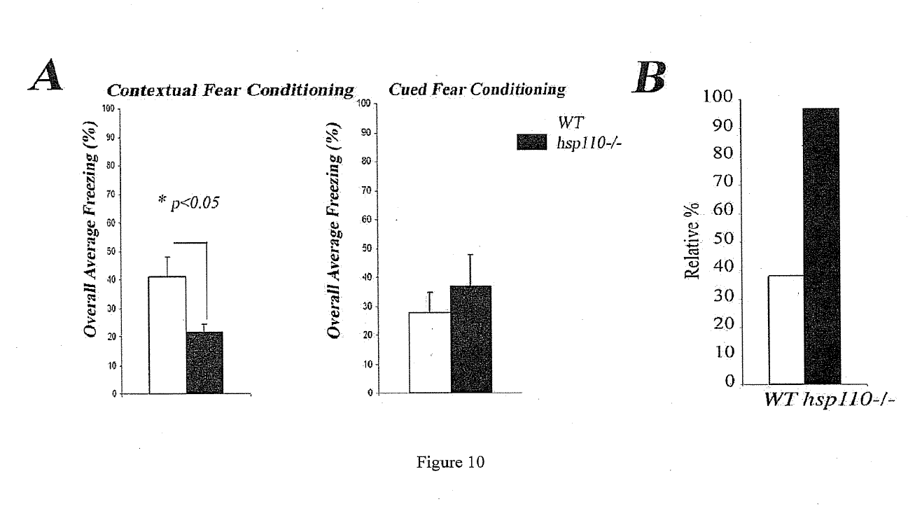

Hsp110^{-/-} mice exhibit behavioral deficit. p-tau has been shown to cause apoptosis of neurons and neurodegeneration (Liou et al., 2003 Nature 424:556-561; Ballatore et al., 2007 Nat Rev Neurosci 8:663-672). Since we observed an age-dependent p-tau accumulation, neuronal death, and NFTs in brain tissue of hsp110^{-/-} mice, we subjected one year old wild-type or hsp110^{-/-} male mice to open field, Y maze/spontaneous alternation, and Novel arm behavioral tests. The data presented in FIG. 8A, FIG. 9 and FIG. 10 indicate that hsp110^{-/-} mice behaved comparably to wild-type mice in open field tests (FIG. 8). The overall rate of alternation was also not significantly different between wild-type and hsp110^{-/-} mice (FIG. 9). However, in Y maze/spontaneous alternation tests, there were significant differences between wild-type and hsp110^{-/-} mice in terms of hsp110^{-/-} mice preference for selecting to enter the right (R) arm (p<0.05 at 2, 5 or 10 minutes) (FIG. 9; bottom). The hsp110^{-/-} mice also exhibit a significant reduction in the contextual fear conditioning test (FIG. 10A, left panel). The contextual fear learning is dependent on the hippocampus, a region of the brain that has been associated with cognitive decline in Alzheimer's disease (Saura et al., 2005 J Neurosci 25:6755-6764). The cued fear conditioning test is independent of the hippocampus and was not significantly altered in hsp110^{-/-} compared to wild-type mice (FIG. 10A, right panel).

Additional behavioral tests were performed with wild-type and hsp110^{-/-} mice at one and half years of age. These tests included limb clasping reflexes and the ability to grasp a bar. In terms of limb clasping, 15-20% of the hsp110^{-/-} mice show periodic limb clasping at this age. However, in the case of the ability of hsp110^{-/-} mice to grasp a bar, we detected deficiency in these mice compared to wild-type mice (FIG. 10B).

These results indicate that hsp110^{-/-} mice show progressive age-dependent behavioral and motor deficits, similar to tau transgenic mice (Lee and Trojanowski, 2001 Neurology 56:S26-30).

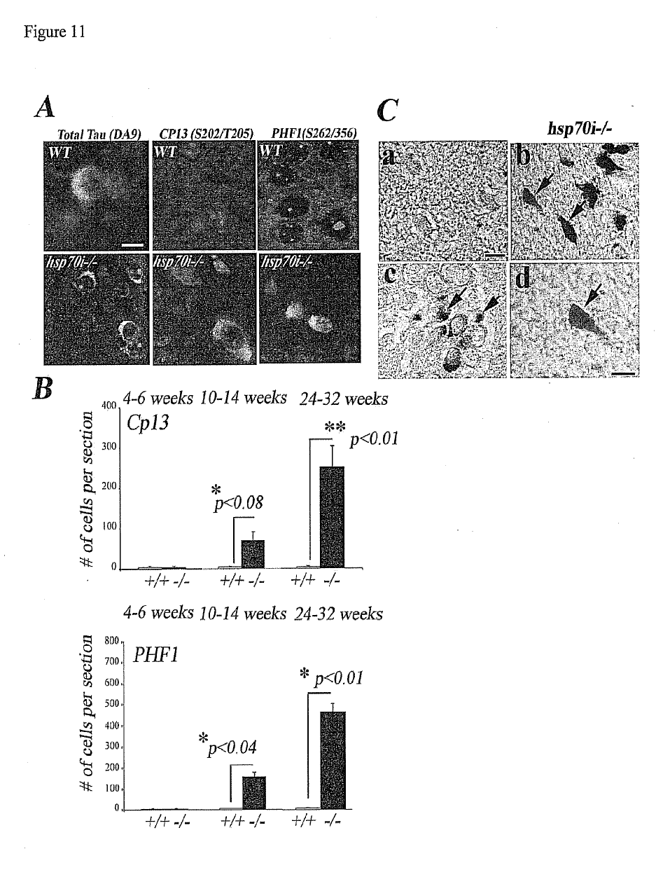

hsp70i-deficient mice exhibit age-dependent accumulation of p-tau. Hsp110 has been shown to be the NEF for the stress-inducible Hsp70.1/70.3 (named as hsp70i) and the constitutively expressed Hsc70 (Dragovic et al., 2006 EMBO J. 25:2519-2528; Polier et al., 2008 Cell 133:1068-1079; Schuermann et al., 2008 Mol Cell 31:232-243). The activities of these proteins have been shown to be required for proper protein folding (Barral et al., 2004 Semin Cell Dev Biol 15:17-29; Bukau et al., 2006 Cell 125:443-451). In order to determine whether the Hsp110 partner, Hsp70, also is required for maintenance of proper phosphorylation of tau, we analyzed the appearance of p-tau in the brain of hsp70i^{-/-} mice at different age groups. The data show that brain tissue sections of hsp70i^{-/-} mice exhibit positive immunoreactivity to p-tau using CP13 or PHF1 antibody, and this increased with age (FIG. 11). There was no cross reactivity using MC1 antibody. In addition, there were a few positive-staining cells expressing NFTs in the hsp70i-brain tissue sections at 32 weeks of age (FIG. 8C, panels b-d) which were not present in wild-type (FIG. 11C, panel a). We also determined PPIase activity in the brain extracts of mice deficient in hsp70i. The data indicate that the level of PPIase activity was reduced in hsp70i^{-/-} brain extracts compared to wild-type, and the reduction in PPIase activity was similar to hsp110^{-/-} mice. Examination of one-year-old male hsp70i^{-/-} mice indicates that 25-30% of these mice exhibit neurodegeneration and limb clasping reflexes.

These data indicate that hsp70i^{-/-} mice also exhibit defects in maintenance of tau phosphorylation state and neurodegeneration.

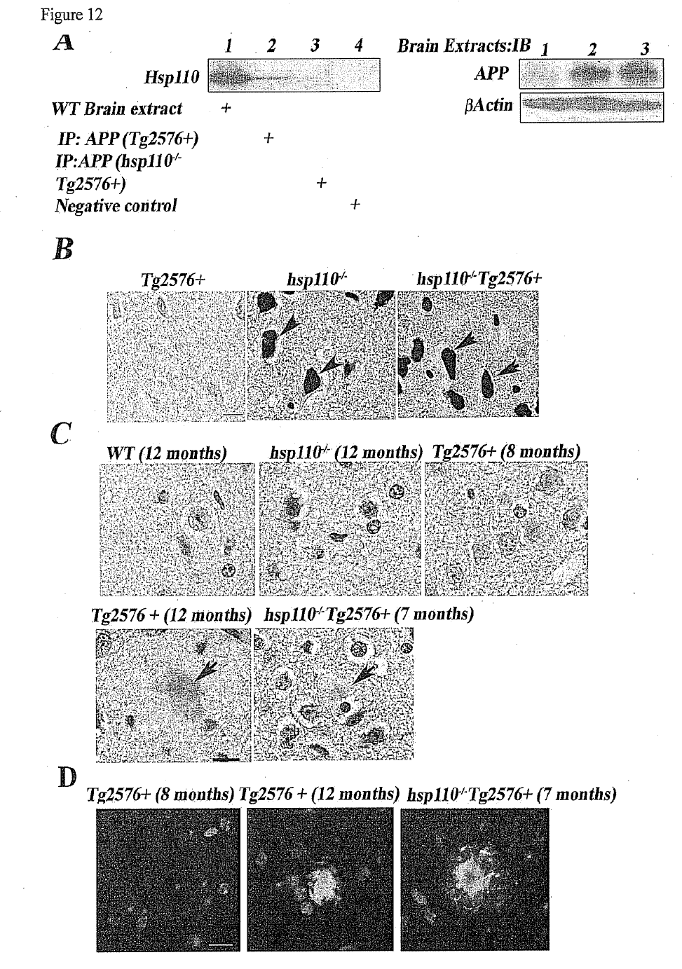

Hsp110^{-/-}Tg2576⁺ mice exhibit accelerated pathology compared to Tg2576⁺ mice. Hsp70 has been shown to be involved in APP processing in cultured cells, since the presence of Hsp70 lowers the toxicity off β-amyloid (Aβ) derived from APP in cells (Kumar, 2007. Hum Mol. Genet. 16:848-64). Hsp70 also protects cultured cells against toxic effects of soluble Aβ42, a critical contributor to formation of plaques in Alzheimer's disease (Kumar et al., 2007 Hum Mol Genet. 16:848-864). Indeed, neuropathological hallmarks of Alzheimer's disease are NFTs that contain both p-tau and Aβ (Ballatore et al., 2007 Nat Rev Neurosci 8:663-672). However, the in vivo evidence that a molecular chaperone may impact APP processing and Aβ accumulation is not known. To determine whether hsp110^{-/-} mice exhibit accelerated pathology when crossed with mice carrying Tg(APPSWE)2576 Kha transgene (also known as Tg2576) that overexpress familial Alzheimer's disease (FAD) APP^{K670N/M671L} mutation (Hsiao et al., 1996 Science 274:99-102), we generated hsp110^{-/-}Tg2576⁺ mice. We first examined whether antibody to APP can pull-down Hsp110 from brain extracts of Tg2576⁺ mice and immunoprecipitation experiments indicate that APP can pull-down Hsp110 (FIG. 12A, lane 2). Left panel in FIG. 12A shows the levels of APP expression in brain extracts of wild-type, Tg2576⁺, and hsp110^{-/-}Tg2576⁺ mice.

Since hsp110^{-/-} mice exhibited NFTs in aged mice, we stained brain tissue sections of Tg2576⁺ or hsp110^{-/-}Tg2576⁺ mice for the presence of NFTs. Tg2576⁺ mice have not been shown to accumulate NFTs, and our data support that (FIG. 12B), however, 6 month-old hsp110^{-/-} Tg2576⁺ mice did show abundant number of cells containing NFTs, and the number of cells was approximately comparable between hsp110^{-/-} and hsp110^{-/-}Tg2576⁺ mice (FIG. 12B). The Tg2576⁺ mice exhibit senile plaques at 12 months of age and older (Hsiao et al., 1996 Science 274:99-102). We therefore, examined whether absence of hsp110 gene leads to appearance of senile plaques in hsp110^{-/-}Tg2576⁺ at a younger age. Analyses of Tg2576⁺ and hsp110^{-/-} Tg2576⁺ brain tissue sections indicate that Tg2576⁺ mice express senile plaques at 12 month of age, while hsp110^{-/-}Tg2576⁺ brain tissue express senile plaques at 7 months of age using Congo Red staining or immunostaining using antibody to Aβ (FIGS. 12C and D). No plaques were detected in 12 months old wild-type, hsp110^{-/-}, or 8 months old Tg2576+ mice (FIG. 12C, upper panels). Unfortunately, Tg2576⁺ mice lacking the hsp110 gene exhibit some lethality at 4-7 months of age. Above data indicate that loss of the hsp110 gene leads to early Aβ production and accelerates neurodegeneration in Tg2576⁺ mice and Hsp110 molecular chaperone plays a critical role in progression of AD.

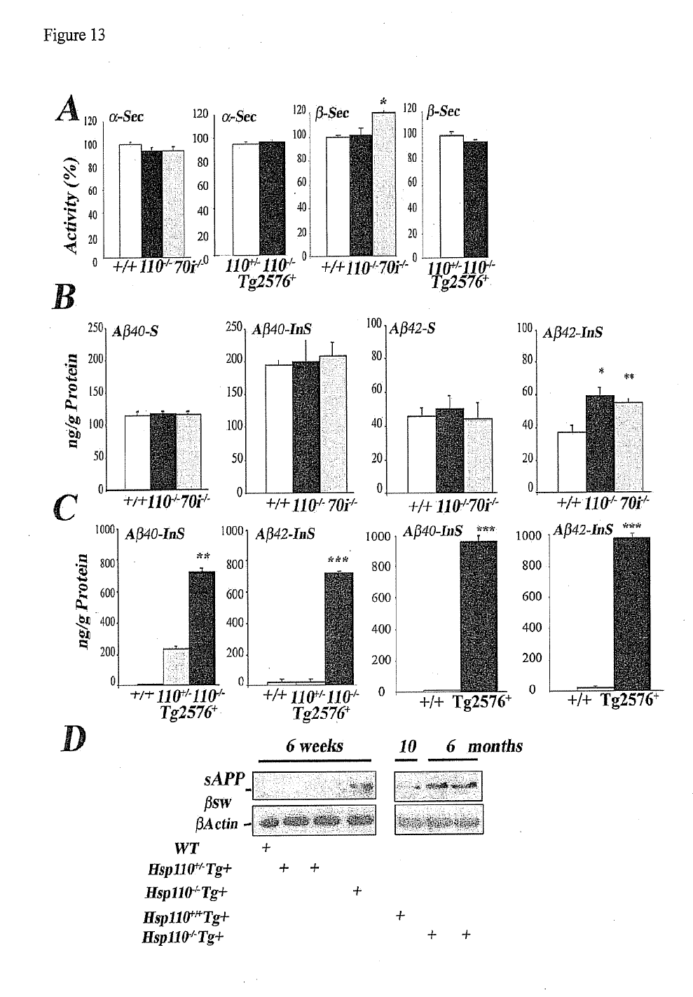

β and β secratase activities and presence of Aβ40 and Aβ42 in brain extracts of hsp110^{-/-} and hsp70i^{-/-} mice. APP is processed by non-amyloidogenic a-secretase at the plasma membrane (Wilquet and DeStrooper, 2004 Curr Opin Neurobiol 14:582-588). APP is processed by α and β secretases at endosomes and other sites (Mattson, 2004 Nature 430:631-639; Wilquet and DeStrooper, 2004 Curr Opin Neurobiol 14:582-588; Pastorino et al., 2006 Nature 440:528-534). In order to determine whether wild-type, hsp110^{-/-}, or hsp70i^{-/-} brain extracts possess comparable levels of α and β secretase activities, 7 months old wild-type, hsp110^{-/-}, and hsp70i^{-/-} brain extracts were used to determine the activities for these enzymes. The data presented in FIG. 13A indicate that the activity of a-secretase was comparable between all genotypes. The activities of β-secretase between wild-type and hsp110^{-/-} brain extracts were also not significantly different, however, β-secretase activity in brain extracts of hsp70i^{-/-} mice was significantly higher than the wild-type mice (p<0.01).

We also determined the levels of mouse Aβ40 and Aβ42 in the soluble and insoluble fractions of brain extracts of wild-type, hsp110^{-/-}, and hsp70i^{-/-} mice. The data indicate that Aβ40 in the soluble fraction, Aβ40 in the insoluble fraction, and Aβ42 in the soluble fraction of brain extracts were comparable between all genotypes (FIG. 13B). However, Aβ42 in the insoluble fraction of brain extracts of hsp110^{-/-} and hsp70i^{-/-} were significantly different than the wild-type mice (FIG. 13B, right panel).

We then asked whether brain tissue extracts derived from hsp110^{-/-}Tg2576⁺ mice express more Aβ40 or the toxic species Aβ42 in the soluble or insoluble brain extracts compared to Tg2576+ mice. The data presented in FIG. 13C indicate that indeed 7 months old hsp110^{-/-}Tg2576⁺ insoluble brain extracts possess significantly higher levels of both Aβ40 and Aβ42 compared to hsp110^{+/-}Tg2576⁺ mice (FIG. 13C, left two lanes). As comparison, one year old Tg2576⁺ also possessed significant amounts of Aβ40 and Aβ42 in the insoluble brain extracts as expected (FIG. 13C, right panels). There were no significant differences between various genotypes in Aβ40 or Aβ42 present in the soluble brain extracts.

Indeed, immunoblotting experiments showed that hsp110 Tg2576⁺ in soluble brain extracts contained sAPPcsw mutant form as early as 6 weeks of age (FIG. 13D), while hsp110^{+/-}Tg2576⁺ did not express this fragment. As expected, both Tg2576⁺ tested at 10 months of age and hsp110 Tg2576⁺ tested at 6 months of age expressed this APP species. Taken together, these data strongly suggest that Hsp110 is critical for delaying insoluble Aβ42 accumulation in this mouse model of AD.

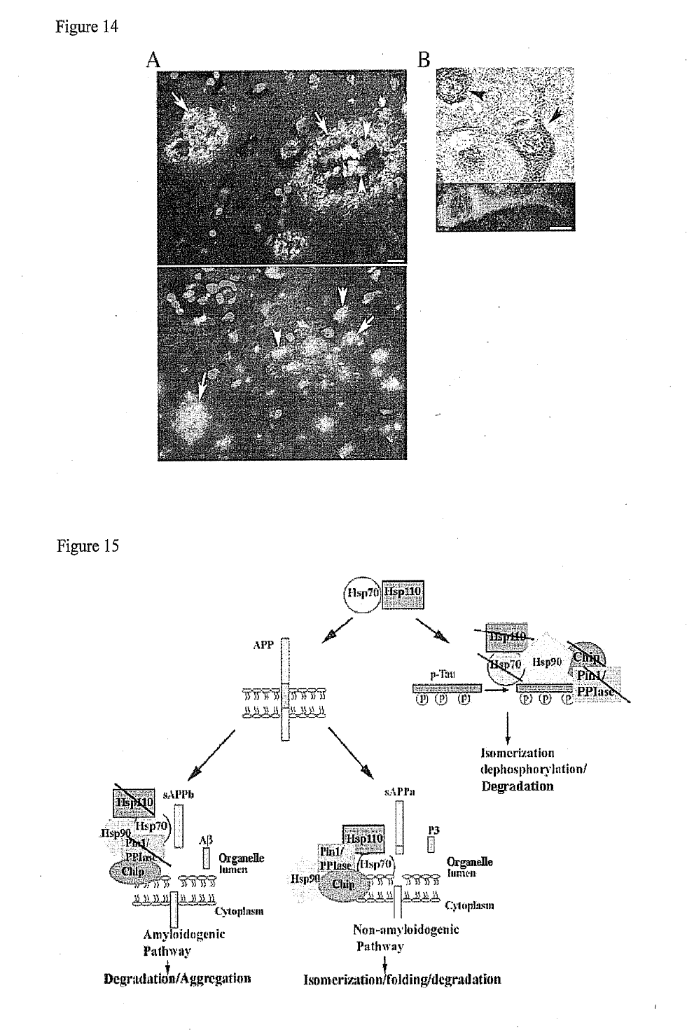

Hsp110 is expressed in Alzheimer's disease and healthy brain tissues of human. Hsp90 and Hsp70 have been shown to colocalize with senile plagues of Alzheimer's brain (Wilhelmus et al., 2007 Mol Neurobiol 35:203-216; Guerreiro et al., 2008 J Alzheimers Dis 13:17-30). As noted before, Hsp110 has been shown to express in neurons; however, whether it colocalizes with Aβ in senile plaques in AD brain has not been investigated. To determine the expression of Hsp110 in human brain tissue, paraffin-embedded sections of healthy human brain and Alzheimer's brain were immunostained using antibody to Hsp110 or using antibodies to Hsp110 and Aβ (FIG. 14A). The data indicate that Hsp110 is expressed in the healthy human brain cells (neurons), and tissue sections of Alzheimer's brain (FIGS. 14A 14B). Interestingly, Hsp110 immunostaining appears in close proximity of Aβ in cells that have intact nuclei (stained with DAPI) (upper panel, arrow heads) and those cells with no obvious nuclei staining (perhaps apoptotic cells), but in proximity of Aβ deposits (FIG. 14 A lower panel, arrow heads).

### Discussion

Tau is a microtubule-associated protein that facilitates microtubule assembly, maintains neuronal integrity, and is involved in axonal transport (Petrucelli et al., 2004 Hum Mol Genet. 13:703-714). Tau predominantly expresses in neurons/axons and is phosphorylated. Tau phosphorylation and dephosphorylation occurs in neurons, which regulates tau association and dissociation with the microtubules which is required for axonal transport (Ballatore et al., 2007 Nat Rev Neurosci 8:663-672). In the disease state, tau remains mainly phosphorylated and detaches from the microtubules, and forms aggregates (Ballatore et al., 2007 Nat Rev Neurosci 8:663-672). Aggregated tau forms neurofibrillary tangles (NFTs) leading to neuronal death. Although the regulation of tau phosphorylation, dephosphorylation, and degradation, is not fully revealed, data indicate that accumulation of phosphorylated tau and its aggregation will lead to neurodegeneration (Trinczek et al., 1995 Mol Biol Cell 6:1887-1902; Billingsley and Kincaid, 1997 Biochem J 323:577-591; Kosik and Shimura, 2005 Biochim Biophys Acta 1739:298-310; Ballatore et al., 2007 Nat Rev Neurosci 8:663-672).

Accumulating evidence indicates a role for molecular chaperones in protein misfolding diseases. Hsp70 and Hsc70 have been shown to associate with phosphorylated tau and together with Chip ubiquitin ligase, they regulate ubiquitination and degradation of tau (Dou et al., 2003 PNAS 100:721-726; Barral et al., 2004 Semin Cell Dev Biol 15:17-29; Dickey et al., 2006 J Neurosci 26:6985-6996; Dickey et al., 2007 J Clin Invest 117:648-658). Brain tissue sections from AD patients as well as other tauopathies such as Pick's disease, corticobasal degeneration, progressive supranuclear palsy and FTDP-17 have been shown to be immunopositive for the molecular chaperones Hsp90 and Hsp70, and their co-chaperones as well as the Chip ubiquitin ligase (Hatakeyama et al., 2004 J Neurochem 91:299-307; Kosik and Shimura, 2005 Biochim Biophys Acta 1739:298-310; Sahara et al., 2007 J Neurosci Res 85:3098-3108). These results have strengthened the notion that molecular chaperones are involved in degradation of abnormal tau. Indeed, chip^{-/-} mice exhibit accumulation of non-aggregated, ubiquitin-negative phosphorylated tau. Chip^{-/-} mice exhibit increased neuronal caspase activity (Dickey et al., 2006 J Neurosci 26:6985-6996). Furthermore, expression of a mutant form of tau, namely P301L, in chip^{-/-} mice leads to accumulation of phosphorylated tau but was not sufficient to induce “pre-tangle” structures in these mice. These results confirmed that phosphorylated tau is prerequisite to tau aggregate formation and that polyubiquitination of tau could be the cause of accumulation of insoluble filamentous tau (Dickey et al., 2006 J Neurosci 26:6985-6996).

Pin1 isomerase, which has not previously been shown in molecular chaperone (e.g., Hsp110) and tau complexes, plays a critical role in the tau phosphorylation state. Pin1^{-/-} mice exhibit tauopathies and neurodegeneration (Liou et al., 2003 Nature 424:556-561). In contrast to chip^{-/-} mice, the pin1^{-/-} mice accumulate phosphorylated tau, NFTs, and exhibit neuronal cell death and motor impairments in aged mice indicating that Pin1 is essential for maintaining the normal tau physiological state. Pin1 isomerization of phosphorylated tau at threonine 231 leads to its dephosphorylation by conformation-specific phosphatase such as PP2A (Liou et al., 2003 Nature 424:556-561).

In this study, we report that the Hsp110 molecular chaperone is essential for the tau phosphorylation state. Hsp110^{-/-} mice exhibit an age-dependent accumulation of p-tau, NFTs, and neuronal death. Hsp110^{-/-} mice at 14-16 months of age do not seem to exhibit as severe a phenotype as pin1^{-/-} mice in terms of the clasping of the hind limb, which is a sign of severe neurodegeneration (Liou et al., 2003 Nature 424:556-561). However, 15-20% of the hsp110^{-/-} mice at 17-20 months of age show periodic hind-limb claspe. In addition, hsp110^{-/-} mice exhibit behavioral abnormalities at 12 months of age and reduced motor activity at 18 months of age. Interestingly, we have found that PPIase activity is lower in brain extracts of hsp110^{-/-} mice compared to wild-type mice. Hsp110 protein also possesses the Pin1 recognition motif (phospho-thr/ser-pro), which is recognized by MPM2 antibody. The level of MPM2 immunoreactivity in the soluble brain extracts increased in pin1^{-/-} mice by 2-3 fold (Liou et al., 2003 Nature 424:556-561). We also observed 1.5-2-fold increase in MPM2 immunoreactivity in hsp110^{-/-} brain extracts, suggesting hsp110 deficiency lowers the Pin1 (or other PPIase) activity, which could lead, at least, in part to accumulation of phosphorylated tau. Hsp110 protein appears to be present in tau immunocomplexes together with tau and Pin1. Hsp110 also appears to directly interact with unphosphorylated tau in vitro. From our results and observations made by others regarding molecular chaperone function in tau phosphorylation and stability, we have added Hsp110 and Pin1 in a hypothetical model (FIG. 15), a portion of which had been shown by others (Liou et al., 2003 Nature 424:556-561; Petrucelli et al., 2004 Hum Mol Genet. 13:703-714; Dickey et al., 20061 Neurosci 26:6985-6996; Pastorino et al., 2006 Nature 440:528-534; Dickey et al., 2007 J Clin Invest 117:648-658) and it is as follows: Hsp70 and its NEF, Hsp110 together with Pin1 are involved in normal phosphorylation and dephosphorylation of tau. Hsp110 interacts with p-tau and Pin1, facilitating isomerization of p-tau by Pin1 and dephosphorylation of p-tau. Phosphorylated tau under normal or disease conditions may be transferred to Hsp90 chaperone machinery for ubiquitination and degradation by Chip ubiquitin ligase and the UPS (Petrucelli et al., 2004 Hum Mol Genet. 13:703-714; Dickey et al., 2007 J Clin Invest 117:648-658). Interestingly, hsp70i-deficient mice also exhibit p-tau accumulation, however, they do not exhibit positive immunoreactivity to MC1 antibody and they develop lower number of cells with NFTs at a comparable age than the hsp110-deficient mice. This could be due to the fact that Hsc70 may overlap in function with Hsp70 in maintaining the tau phosphorylation state. Hsp110 homologues less likely to have overlapping function since for example, the Hsp110 family member, Apg1 (Hspa41 or Osp94) expresses mainly in the testis, and Apg1^{-/-} mice exhibit increased infertility (Held et al., 2006 Mol Cell Biol 26:8099-8108). Both Apg1 and Apg2 have been reported to also express in the CNS, but it is anticipated that may have different functions than the Hsp110 (Easton et al., 2000 Cell Stress Chaperones 5:276-290; Yoneyama et al., 2008 Neuropharmacol 55:693-703).

Whether or not the phenotype observed in hsp110-deficient mice reflects in part a lower PPIase activity is not known. However, it is clear that molecular chaperones such as Hsp110 and Hsp70 are critical for the tau-Pin1 complexes to keep tau proper phosphorylation state. It should be noted that Hsp110 deficiency reduces and does not entirely abolishes PPIase activity as is the case for pin1^{-/-} mice (Pastorino et al., 2006 Nature 440:528-534).

Interestingly, crossing hsp110^{-/-} mice with Tg2576⁺ mice leads to appearance of Aβ plaques in younger hsp110^{-/-}Tg2576⁺ compared to Tg2576⁺ mice. Normally, in Tg2576⁺ mice plaques appear around one year of age, while we could detect plaques in hsp110^{-/-}Tg2576⁺ mice as early as 7 months of age. We have also found that Hsp110 interacts with APP (FIG. 12). In addition, hsp110^{-/-}Tg2576⁺ mice exhibit increase in amyloidogenic APPβ sw processing that could potentially lead to Aβ42 accumulation as early as 6 weeks of age. And when hsp110^{-/-} mice were crossed with Tg2576⁺ mice they accumulate insoluble Aβ40 and Aβ42 at significantly accelerated pace compared to hsp110^{+/-}Tg2576⁺ mice. Hsp110^{-/-} and hsp70i^{-/-} mice also accumulate insoluble 442 tested at one year of age compared to wild type mice. These data suggest that absence of hsp110 gene leads to defect in APP processing and Aβ accumulation. Interestingly, the activities of both α and β secretases in hsp110^{-/-} brain extracts are at comparable levels as the wild-type mice. In vitro cell culture studies suggest that Hsp90 and Hsp70 together with Chip ubiquitin ligase interact and affect the metabolism of βAPP (Kumar et al., 2007 Hum Mol Genet. 16:848-864). Additionally, data suggest an interaction between Chip, Hsps, and Aβ42 and that presence of these molecules reduces buildup of Aβ42 (Kumar et al., 2007 Hum Mol Genet. 16:848-864). Comparably, Pin1 overexpression has been shown to decrease Aβ secretion and reduction in Pin1 increases Aβ secretion (Pastorino et al., 2006 Nature 440:528-534). Mice deficient in Pin1 overexpressing APP mutant exhibit increase in amyloidogenic processing of APP and generation of Aβ42. Indeed, Pin1 isomerization of APP controls Aβ production and APP processing (Pastorino et al., 2006 Nature 440:528-534). Therefore, we envision that nonamyloidogenic APP processing and Aβ production and APP degradation likely requires at least in part (or in specific cellular compartments) Hsp70, Hsp110, Hsp90, their cochaperones, Chip, and Pin1 (or other PPIases) (Dickey et al., 2006 J Neurosci 26:6985-6996; Pastorino et al., 2006 Nature 440:528-534; Dickey et al., 2007 J Clin Invest 117:648-658; Kumar et al., 2007 Hum Mol Genet. 16:848-864). Deletion of either Pin1 or Hsp110 (or presumably Hsp70i) in vivo leads to amyloidogenic process of APP and toxic Aβ42 accumulation. There is a possibility that Hsp70 (Kumar, 2007. Hum Mol. Genet. 16:848-64) and Hsp110 (FIG. 12) as they interact with APP play additional roles in APP folding and could possess independent function other than APP degradation in conjunction with Pin1 (or other PPIases) as well and this will need to be determined. The model presented in FIG. 15 depicts our findings as well as previous data from other laboratories. FIG. 15 shows the absence of Hsp110, Hsp70, Chip, or Pin1 gene leads to hyperphosphorylated tau in brain tissue. The product of these genes may be involved in isomerization, dephosphorylation, and/or degradation of tau or p-tau (Liou et al., 2003 Nature 424:556-561; Dickey et al., 2006 J Neurosci 26:6985-6996; Petrucelli et al., 2004 Hum Mol Genet. 13:703-714). Comparable molecular chaperone complexes appear to affect APP processing in non-amyloidogenic pathway (Kumar et al., 2007 Hum Mol Genet. 16:848-864; Wilhelmus et al., 2007 Mol Neurobiol 35:203-216). Absence of Hsp110 or Pin1 in vivo leads to activation of amyloidogenic pathway leading to accumulation of insoluble Aβ42 (Pastorino et al., 2006 Nature 440:528-53.) This model suggests a critical role for molecular chaperones including Hsp110 in tau phosphorylation and APP processing and Aβ production.

In this example, we have provided genetic evidence that lack of hsp110 in mice is associated with tauopathy and neurodegeneration, suggesting that molecular chaperones are critical in neurodegenerative diseases such as AD or other tauopathies. Hsp110-deficient mice provide a good model system to define mechanisms involved in APP processing and Aβ plaque accumulation as well as state of tau phosphorylation.

### Example 2

### Generation and Analysis of Hsp70.1 and Hsp70.3 (Hsp70i) Deficient Mice

High levels of Hsp90, Hsp70 and Hsp27 expression either individually or in combination have been widely reported in human tumors, especially those of epithelial origin. Indeed this has been suggested to be of prognostic value in cancer in that over-expression of Hsps correlated with poor patient outcome in certain tumors (Jaattela, 1995 Int J Cancer 60:689-693; Vargas-Roig et al., 1997 Cancer Detect Prey. 21:441-451; Nanbu et al., 1998 Cancer Detect Prey 549-555; Mosser and Morimoto, 2004 Oncogene 23:2907-2918; Jaattela, 2004 Oncogene 23:2746-2756; Ciocca and Calderwood, 2005 Cell Stress and Chaperones 10:86-103). Therefore, based on the prediction that high levels of molecular chaperones are protective against cell death and increase cell survival against toxic insults such as chemotherapy agents, targeting Hsp expression or function has been suggested as an effective anti-cancer strategy for many tumor types (Kamal et al., 2003 Nature 425:407-410; Whitesell and Lindquist, 2005 Nat Rev Cancer 5:761-772; Guo et al., 2005 Cancer Res. 65:10536-10544). However, the diverse functions of Hsps in oncogenesis are as yet far from understood. In addition, little is known about the molecular mechanisms responsible for over-expression of Hsps in cancer cells and the relative contributions of the Hsps in tumor development. To address these important issues we have generated mutant mice, allowing us to dissect in vivo the role of three major molecular chaperones, Hsp70i, Hsc70, and Hsp25. Studies in the present application are likely to be valuable in delineating a number of mechanisms involving Hsps, thought to play a key role in cancer transformation and tumor growth. The fact that Hsp90 inhibitors are currently showing promise in clinical trials of cancer treatment support the premise of this proposal that a better understanding of the fundamental cellular processes in which Hsp70s and Hsp25 molecular chaperones engage to promote tumor growth may help to develop more effective strategies to modulate specific chaperone-dependent host pathways as a therapeutic approach to combat human cancers and other relevant diseases.

### Genomic Organization of the Hsp70 and Hsp27 Families

In mammalian cells, the stress response involves the induction of five major Hsp families, classified according to their molecular mass, namely small Hsps (exemplified by Hsp27), Hsp60, Hsp70, Hsp90 and Hsp104 (Feder and Hofmann, 1999 Ann Rev. Physiol. 61:243-282; Smith et al., 1998 Pharmacol Rev 50:493-513).

Hsp70i. The inducible murine hsp70i (hsp70.1 and hsp70.3) genes encode identical proteins of 641 amino acids. This inducible expression is regulated by Hsfs (−1, -2 and -4), which bind to heat shock elements (nGAAn) repeats in the promoter regions of the hsp70i genes (Morimoto, 1998 Genes and Development 12:3788-3796; Pirkkala et al., 2001 FASEB J 15:1118-1131). However, the hsp70i genes also contain putative sites for a number of other transcription factors, including Sp1 (Perry et al., 1996 Gene 146:273-278; Bevilacqua et al., 1997 Nucleic Acids Res 25:1333-1338; Zakari et al., 1988 Mol Cell Biol 8:2925-2932; Hunt and Calderwood, 1990 Gene 87:199-204). Recent studies with mutant mice confirmed the role of Hsf1 as a major trans-activator of Hsp70i expression in response to increased stress (Xiao et al., 1999 EMBO J. 18:5943-5952; Zhang et al., 2002 J Cell Biochem 86:376-393). However, no apparent contribution for Hsf2 or Hsf4 in Hsp70i expression was observed. Constitutive tissue specific expression of Hsp70i, including expression in blood vessels, is not regulated by Hsfs (-1, -2 or -4).

Hsc70. Hsc70 is involved in several aspects of normal cellular protein trafficking, including folding of nascent polypeptide chains on polyribosomes (Beckmann et al., 1990 Science 248:850-854), uncoating of clathrin-coated vesicles (Chappell et al., 1986 Cell 3-13), and transport of proteins into cellular organelles (Deshaies et al., 1988 Trends Biochem Sci. 13:384-388). Hsc70 is expressed ubiquitously with very high levels in the nervous system.

Hsp27 Chaperones. Mammalian small stress proteins (Hsp27, Hsp20, HspB3, MKBP/HspB2, HspB8, HspB9, cvHsp, αA- and αB-crystallin) share an 80-100 amino acid residue α-crystallin domain in the C-terminal region, and are particularly abundant in muscles and heart. In contrast to the other major chaperones, Hsp27 is ATP independent, yet can efficiently associate with unfolded proteins and maintain them in a folding-competent state. In unstressed cells Hsp27 levels are generally low, being predominantly comprised of large oligomeric units of up to 800 kDa made up of six tetrameric complexes of Hsp27. During the stress response increased Hsp27 expression is preceded by a phosphorylation-induced reorganization of these Hsp27 multimers (Haslbeck and Buchner, 2002 Prog Mol Subcell Biol. 28:37-59). Such stress-induced phosphorylation is catalyzed by MAPKAP kinases-2 and 3, which in turn are activated through phosphorylation by p38 MAP kinase (Landry and Huot, 1999 Biochem Soc Symp. 64:79-89; Rouse et al., 1994 Cell 78:1027-1037). Heat-induced nuclear protein aggregation is resolved more rapidly during recovery from severe stress exposure in cells overexpressing Hsp27 (Kampinga et al., 1994 Biochem Biophys Res Commun 204:1170-1177). Hsp27 binds to Factin and can prevent disruption of the cytoskeleton resulting from either heat stress or cytochalasin D-induced disruption of actin filaments (Guay et al., 1997 J Cell Sci 110(Pt3):357-368). The intron-exon structure of the murine hsp25 locus is similar to that of human hsp27, and the transcription start points of the genes are located at similar sites.

### Role of Hsp70 and Hsp27 Molecular Chaperones in Tumor Development

Hsp70 and Hsp27 regulate apoptosis. Apoptosis is the best-defined cell death program counteracting tumor growth, and defects in signaling pathways leading to programmed cell death (PCD) are major hallmarks in cancer development. Many studies have revealed that PCD can occur by both caspase dependent apoptotic death pathways and in the complete absence of caspase activation (Jaattela, 2004 Oncogene 23:2746-2756). Several proteins may promote tumorigenesis by inhibiting apoptosis, but of special relevance are those expressed in primary tumors including members of the Bcl-2 protein family, Hsp70 and Hsp27 and Survivin (Jaattela, 1999 Exp Cell Res 248:30-43; Sreedhar and Csenuely, 2004 Pharmacol Ther 101:227-257). Understanding the function of Hsp70 and Hsp27 in cancer cell apoptosis may offer new modalities for selective manipulation of the sensitivity of cancer cells to therapy.

Hsp70. The molecular mechanisms underlying the tumorigenic potential of Hsp70 (Hsp70i and Hsc70) are as yet unclear. In vitro studies have suggested that Hsp70 may directly interfere with the apoptosis signaling machinery by binding to the apoptotic protease-activating factor-1 (Xanthoudakis and Nicholson, 2000 Nat Cell Biol 9:E163-165) or apoptosis-inducing factor (Beere et al., 2000 Nature Cell Biology 2:469-475; Ravagnan et al., 2001 Nature Cell Biology 3:839-843) and thereby inhibit the apoptosome-mediated activation of caspases or apoptosis-inducing factor-induced nuclear changes, respectively. However, most studies using cellular death models suggest that Hsp70-mediated inhibition of caspase-dependent PCD may occur upstream of mitochondrial outer membrane permeabilization and apoptosome formation (Mosser et al., 2000 Mol Cell Biol 20:7146-7159; Gabai et al., 2002 Mol Cell Biol 22:3415-3424; Nylandsted et al., 2004 J Exp Med 200:425-435). Furthermore, Hsp70 can effectively rescue cells from caspase-independent PCD induced by TNF, heat shock, serum starvation, or oxidative stress, and the depletion of Hsp70 induces caspase-independent apoptosis in various human tumor cell lines (Nylandsted et al., 2000 PNAS 97:7871-7876; Nylandsted et al., 2002 Cancer Res 62:7139-7142). Thus, the main mechanism by which Hsp70 confers a survival advantage to tumor cells appears to be inhibition of the permeabilization of lysosomal membranes and/or membranes of other vesicles containing cathepsins induced by diverse stimuli, including cytokines, anticancer drugs, ionizing-radiation, and oxidative stress.

Hsp27. Hsp27 mediates protection against stress through maintaining normal cell function by stabilizing the cytoskeleton, facilitating repair or removal of damaged proteins, and inhibiting components of both stress and death-receptor induced apoptotic pathways (Mosser and Morimoto, 2004 Oncogene 23:2907-2918, Jaattela, 1999 Exp Cell Res 248:30-43; Sreedhar and Csermely, 2004 Pharmacol Ther 101:227-257, Gamido et al., 2001 Biochem Biophys Res Commun 24:433-442; Paul et al., 2002 Mol Cell Biol 22:816-834; Concannon et al., 2003 Apoptosis 8:61-70). Various mechanisms have been proposed to account for the ability of Hsp27 to protect stressed cells from apoptotic death, including decreasing generation of radical oxygen species, directly interacting with cytochrome c released from mitochondria, or facilitating activation of the ubiquitin-proteasome pathway. In addition Hsp27 can inhibit apoptosis by regulating signaling pathways upstream of apoptosome formation by binding Akt, an interaction necessary for Akt activation in stressed cells (Konishi et al., 1997 FEBS Lett 410:493-498; Rane, et al., 2003 J Biol Chem 278:27828-22735). Hsp27 shares several properties with Hsp70. Expression of both stress proteins inhibits apoptosis, induces resistance to most chemotherapeutic agents, and enhances tumorigenesis in rodents. However, several differences between the two chaperones have been identified: Hsp70 function depends on ATP hydrolysis, whereas Hsp27 does not; Hsp70 is an early responsive gene, whereas Hsp27 is a late responsive one; and their anti-apoptotic effects involve distinct molecular mechanisms (Paul et al., 2002 Mol Cell Biol 22:816-834; Concannon et al., 2003 Apoptosis 8:61-70, Jakob et al., 1993 J Biol Chem 268:1517-1520; Ehrnsperger et al., 1997 EMBO J. 16:221-229; Beissinger and Buchner, 1998 Biol Chem 379:245-259). Consequently, these proteins are regarded as complementary protective proteins.

Molecular chaperones in angiogenesis and vasculogenesis. Blood vessel development is a highly organized process that is initiated by differentiation of pluripotent stem cells into endothelial cells, which proliferate, migrate, and eventually form the vasculature (Risau, 1997 Nature 386:671-674; Yancopoulos et al., 2000 Nature 407:242-248; Oettgen, 2001 Circ Res 380-388; Carmeliet, 2003 Nat Rev Genet. 4:710-720). Maturation of these primitive endothelial tubes into fully developed blood vessels requires the recruitment of surrounding pericytes and their differentiation into vascular smooth muscle cells. Many of these events that occur during vasculogenesis (the formation of blood vessels de novo from precursor cells), also occur during angiogenesis (the formation of new blood vessels from pre-existing vasculature). Angiogenesis involves a complex series of reciprocal interactions between endothelial cells of developing blood vessels and neighboring cells. Perivascular mesenchymal cells provide endothelial cells with signals such vascular endothelial growth factor (VEGF), angiopoietin 1, platelet-derived factor and transforming factor β. This process is recapitulated during angiogenesis in several pathological conditions, including solid tumor growth (Folkman, 2003 Curr Mol Med 3:643-651; Bergers and Benjamin, 2003 Nat Rev Cancer 3:401-410).

Understanding the dynamic interaction between cancer cells and their microenvironment, and application of this knowledge to detection, diagnosis, prevention, and treatment of cancers is a major goal in the cancer biology field. Mounting evidence suggests that the “tumor microenvironment” is populated with a variety of different cell types, including elements of the blood and lymphatic systems, which play a pivotal role in cancer development and can influence the delivery and processing of therapeutic agents to the tumor. The role of angiogenesis in growth and development of cancers in experimental and human tumors has been well established (Folkman, 2003 Curr Mol Med 3:643-651; Neri and Bicknell, 2005 Nat Rev Cancer 5:436-446; Tozer et al., 2005 Nat Rev Cancer 5:423-435; Joyce, 2005 Cancer Cell 7:513-520). Tumors secrete VEGF and other angiogenic factors to regulate vascular development required for tumor growth and survival. VEGF targets a set of tyrosine kinase cell surface receptors (VEGFR1-3) on endothelial cells, and pluripotent endothelial precursor cells (EPC) in the bone marrow (BM), recruiting them into the tumor angiogenesis process by activating an endothelial lineage differentiation program (CD31/PECAM-1, VE-cadherin, and TIE-2 activation) culminating in their release into the circulation (Pugh and Ratcliffe. 2003 Nat Med 9:677-684; Rafii and Lyden. 2003 Nat Med 9:702-712). Regulation of VEGFR and TIE-2 expression during differentiation is mediated by several transcription factors including Ets, GATA factors, HIF-1α and HIF-2α. While a substantial body of evidence supports antiangiogenic therapy as a treatment modality using small molecule inhibitors, this strategy is still in its infancy (Underiner et al., 2004 Curr Med Chem 11:731-745). The current application proposes to explore the potential contribution of molecular chaperones in angiogenesis, which should generate important information that may aid in developing improved antiangiogenic therapies.

Generation of Hsp70.1 and Hsp70.3 (Hsp70i) deficient mice. To perform studies on acquired thermotolerance and protection from different stress situations, we generated double Hsp70i−/− mice. As the hsp70.1 and hsp70.3 genes are located only 10 Kb apart on the same chromosome, mice carrying both targeted genes cannot be realistically obtained by intercrossing of single deficient mice. Therefore, we devised dual strategies to approach this issue: (1) The transallelic transgenic targeted meiotic recombination (TAMERE) protocol described by Denis Duboule (Vidal et al., 1998 Mol Reprod Dev 51:274-280; Kmita et al., 2002 Nature 420:145-150) has been used to achieve deletion of both Hsp70 genes on the same chromosome. To this end we have crossed mice heterozygous for both hsp70.1 and hsp70.3 targeted alleles and expressing the Sycp1/Cre transgenic with C57BL/6 mice to obtain animals disrupted in both hsp70i genes. However, despite extensive breeding, we have been unsuccessful in obtaining the desirable recombinant genotype. (2) A gene-deletion strategy involving replacement of the hsp70.1 and hsp70.3 coding sequences, including the intergenic region, with the LacZ reporter gene was pursed as an alternative strategy.

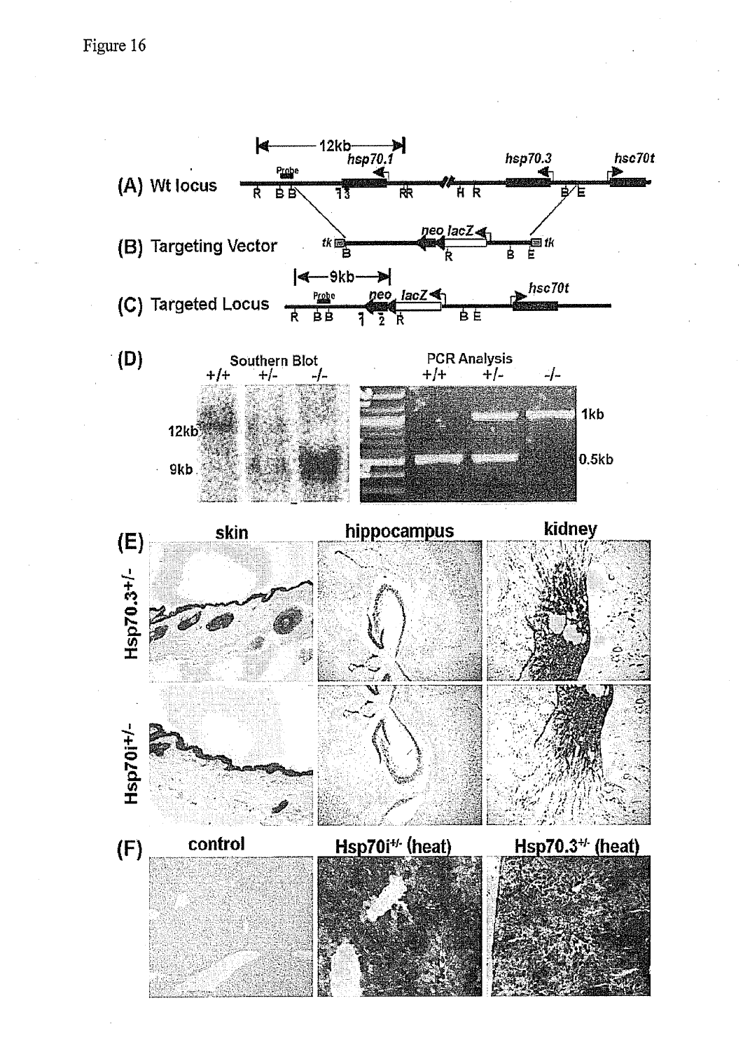

As shown in FIG. 16, this approach results in disruption of both functional hsp70i genes as well as producing an in situ reporter for the activity of Hsfs and other relevant transcription factors. Offspring mice with germ-line transmission of the deleted hsp70i genes were interbred and homozygous mutant mice were born with the expected Mendelian frequency, indicating that Hsp70i is not essential for embryo survival, confirming published observations. The fact that our mice have a reporter gene in the locus to trace tissue-specific Hsp70i expression provides a major advantage over hsp70i−/− mice generated by other groups (Hampton et al., 2003 Am J Physiol Heart Circ Physiol 285:H866-874; Hunt et al., 2004 Mol Cell Biol 24:899-911). Whilst detailed phenotypic studies of these mice are underway, initial analyses suggest that the intergenic region separating the hsp701 genes is dispensable for tissue-specific constitutive or heat-induced expression of hsp70.3 (FIG. 16). Furthermore, we observe hsp70.3-LacZ reporter expression in the embryonic vasculature of hsp70i^{-/-} or hsp70i^{+/-} mice consistent with results from hsp70i^{+/-}-LacZ-reporter mice as described below.

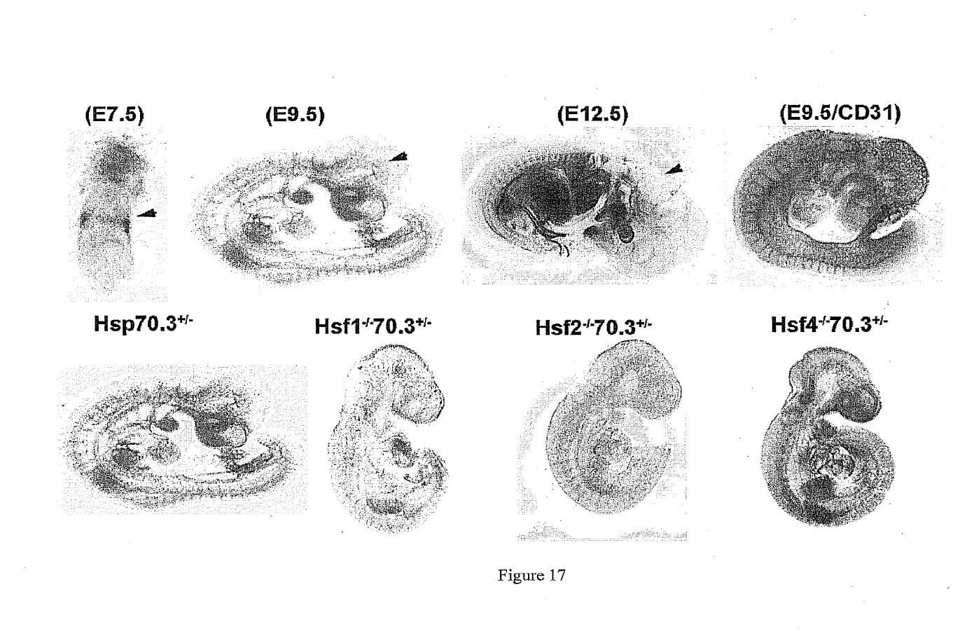

Hsp70i expression is restricted to endothelial cells during embryonic development and tumor growth marks and guides blood vessel formation. Detailed analysis of hsp70i expression during embryonic development by visualizing LacZ expression in hsp70.1^{+/-} or hsp70.3^{+/-} mice revealed that at an early embryonic stage (E7.5), expression of hsp701 was restricted to the chorionic plate, where the progenitors for blood vessel formation reside. In mid-gestation (E9.5 and E12.5), a remarkably intense staining was visualized in the entire vascular system including the umbilical cord. Such hsp70i expression in the vascular system was down regulated during late-gestation. Further studies performed on Hsp70.1^{+/-} or Hsp70.3^{+/-} mice deficient in hsf1, hsf2, or hsf4 revealed that the hsp70i expression in the vasculature was not regulated by Hsfs. The same conclusion can be drawn for constitutive hsp70i gene expression in other tissues. A demonstration of hsp70.3 expression in embryonic blood vessels (endothelial cells as revealed by whole mount staining of embryos for CD31 at E9.5) is presented below (FIG. 17). Similar results were obtained for hsp70.1 gene expression. The interesting possibility that Hsfs may function synergistically in regulating tissue specific expression of hsp70.1 or hsp70.3 in embryonic tissues including blood vessels or in adult mice has been explored in a recent study, which revealed that the overall Hsp70i basal expression level in particular tissues was not detectably altered in Hsf-1^{-/-}2^{-/-}4^{-/-} mice. In addition we have detected similar expression levels for Hsc70 and Hsp25 (except in the lung) in tissues from Hsf-1^{-/-}2^{-/-}4^{-/-} mice compared to wild type B6-controls. Thus, these data underscore the distinct Hsf-independent transcriptional programs by which basal hsp701 expression is regulated.

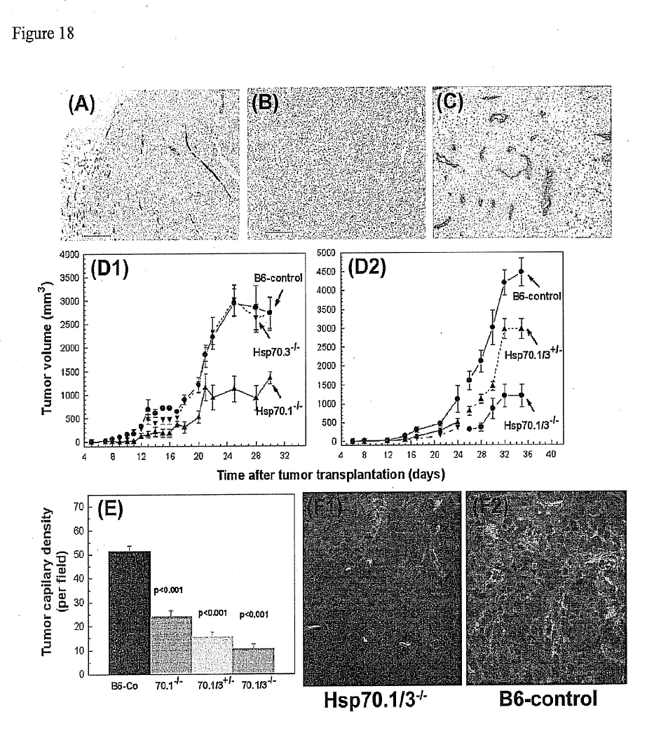

As tumors proliferate, they recruit host-derived blood vessels. Tumour angiogenesis was studied in a well-established mouse model. Results with tumor cells transplanted subcutaneously in Hsp70^{+/-}-LacZ reporter mice revealed striking Hsp-expression in tumor infiltrating blood vessels detectable only during the onset of tumor growth (shown for Lewis Lung Carcinoma (LLC) cells in FIG. 18). LacZ staining was detected in the majority of endothelial cells throughout the developing tumor vasculature as determined by CD31 staining of consecutive sections. In further studies we have observed that tumor growth was substantially retarded when LLC cells were transplanted into Hsp70i^{-/-}, Hsp70i^{+/-} or Hsp70.1^{-/-} mice, but not into Hsp70.3^{-/-} mice, compared to C57BL/6 control mice with intact Hsp70i genes (FIG. 18D). Note that Hsp70^{-/-} mice used for this analysis were on a C57BL/6 background (back-crossed for >12 generations). A consistent histological finding in tumors of Hsp70i−/− was a markedly lower density of microvessels (FIGS. 18E and 18F). Based on these data, we postulate that Hsp70i deletion impairs tumor microvessel proliferation resulting in tumor retardation. Clearly, further analysis is required (for example studying tumor growth in BM reconstituted irradiated recipient mice), to determine whether impaired recruitment of pre-existing and/or BM derived endothelial precursor cells to the tumor vasculature is responsible for this reduced tumor growth. However, our preliminary studies support a basic tenet of this application, namely that ablation of Hsps (Hsp70i, Hsc70, Hsp25) will reduce tumor growth and sensitize developing endothelial cells to stress stimuli including radiation and heat.

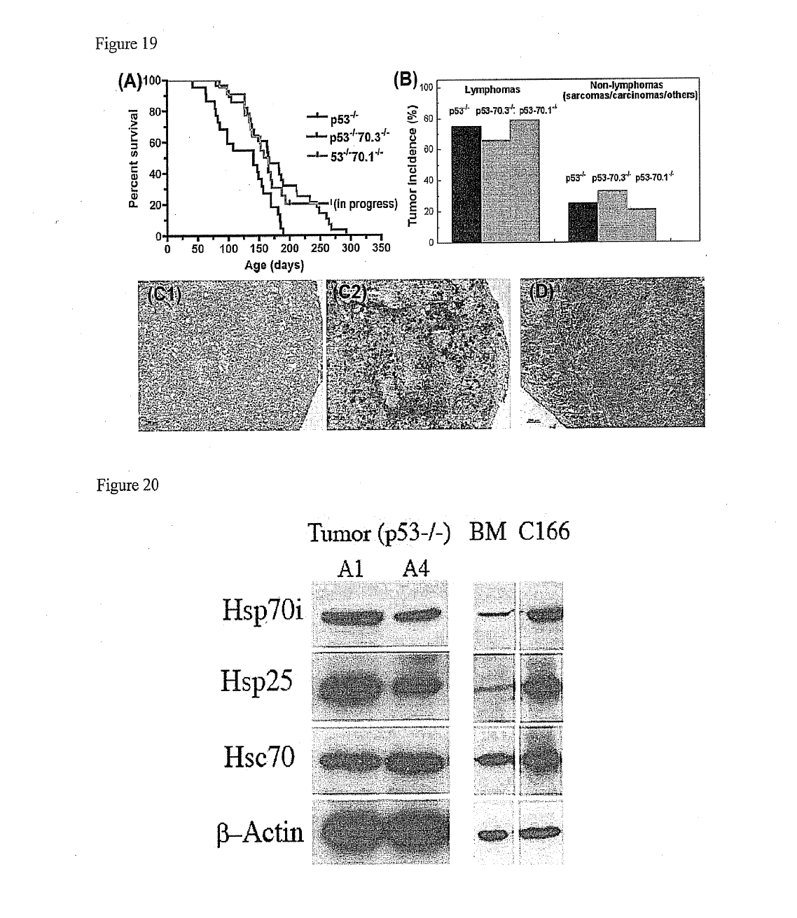

Tumor induction and progression in Hsp70i^{-/-}-p53^{-/-} mice. We have determined whether loss of Hsp70.1 or Hsp70.3 function plays a significant role in development and progression of cancer in conjunction with genetic alteration of p53 tumor suppressor gene expression. The hypothesis guiding these experiments is that Hsp70i deficiency alters the growth or prevalence of tumors arising in p53^{-/-} mice. As shown in FIG. 19, loss of Hsp70.3 or Hsp70.1 function prolonged tumor free survival in p53 deficient mice. However, we did not observe an alteration in the spectrum of tumors that arose in Hsp70.1^{-/-}p53^{-/-} or Hsp70.3^{-/-}p53^{-/-} versus p53^{-/-} mice. This result differs from the findings made with Hsf1 mice, in which loss of Hsf1 function did not prolong tumor free survival but altered the spectrum of tumors that arose in p53 deficient mice. However, this may not be surprising in light of potential compensatory effects through the intact hsp70.1 or hsp70.3 genes in the single hsp70 deficient mice. Definitive studies to address this issue using Hsp70i^{-/-} double deficient mice are proposed in the following examples.

Hsp expression in endothelial cells and tumors. Hsps are expressed in several tumor types and we have observed that spontaneously arising lymphomas in p53^{-/-} mice express Hsp70i, Hsc70 and Hsp25 at high levels (FIG. 20). In addition, these Hsps are expressed in endothelial cells, as revealed by proteomic analysis of primary human umbilical vein endothelial cells (HUVEC) (Bruneel et al., 2003 Proteomics 3:714-723). To confirm these results we have analyzed expression of Hsps in different mouse endothelial cells. A representative demonstration for mouse endothelial C166 cells (FIG. 20), demonstrates that these molecular chaperones are prominently expressed under normal conditions. Finally, we also observed expression of these molecular chaperones under normal conditions in BM cells and have obtained preliminary evidence that lineage negative cells are the major population expressing Hsp70i and Hsp25 whereas Hsc70 is ubiquitously is expressed in every cell type.

In summary, this example suggests that Hsp70 family members (Hsp70i and Hsc70) with common functional properties may act synergistically in tumor development by regulating components of the tumor environment and influencing tumor cell survival and growth. Hsp25, which shares common functions but also possesses unique properties compared to Hsp70 chaperones, may also positively influence tumor growth. Extensive studies are proposed in following examples, to evaluate the contribution of these molecular chaperones to tumor biology in animal models.

### Example 3

### Wild-Type Mice Exhibit Sensitivity to Traumatic Brain Injury (TBI)-Induced Edema

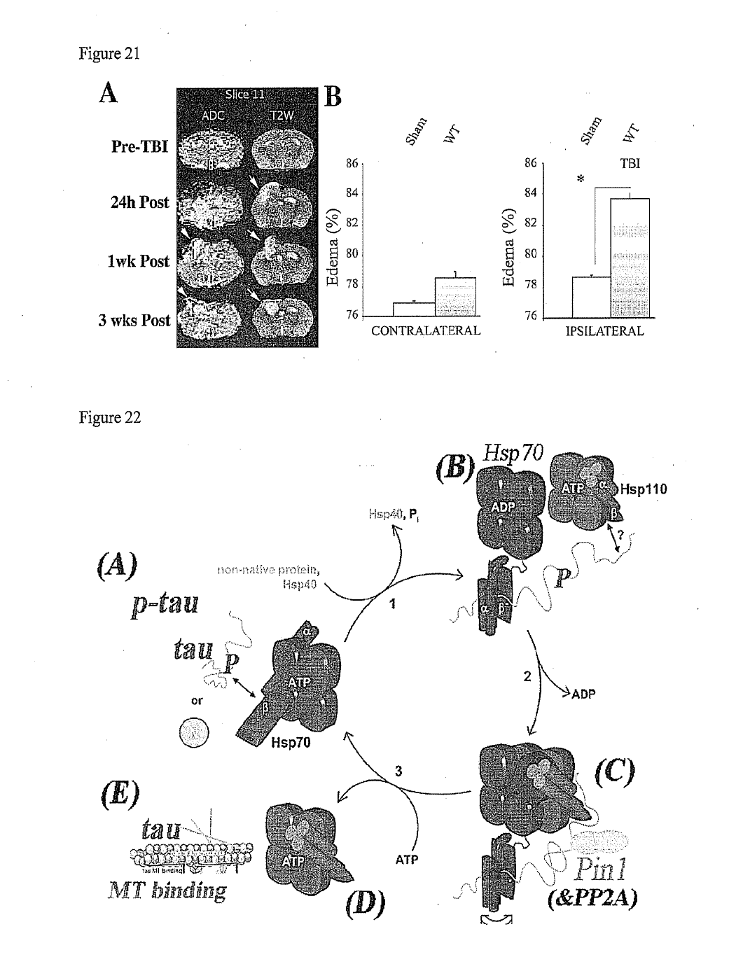

We have subjected wild-type to TBI and performed MRI analyses after 24 hours, 1 or 3 weeks. FIG. 21 shows imaging brain edema following TBI in mice. TBI was applied using a 2 mm diameter pneumatic piston (Air-Power, Inc. High Point, N.C.) (Griebenow et al., 2007 J Neurotrauma 24:1529-1535; Zweckberger et al., 2006 J Neurotrauma 23:1083-1093). The impactor is discharged at 6.8+/−0.2 m/s with the head displacement of 3 mm. The MRI shows significant areas of edema following TBI (FIG. 21A). We also subjected both wild-type and hsp110^{-/-} mice to TBI. In FIG. 21B, wild-type male mice (n=5) were subjected to TBI and 24 hours later, brain water content was estimated in a 3 mm coronal tissue section of the ipsilateral cortex (or corresponding contralateral cortex that were not treated), centered on the impact site. Tissues were immediately weighed (wet weight), then dehydrated at 65° C.) (Miao et al., 2001 Cancer Res 61:7830-7839; Troyanovsky et al., 2001 J Cell Biol 152:1247-1254). The samples were reweighed 48 hours later to obtain a dry weight. The percentage of water content in the tissue samples were calculated using the following formula: {(wet weight−dry weight)/wet weight}×100. *p<0.001. The data for wild-type mice show significant edema development 24 hours following TBI compared to sham control (anesthetized only) (FIG. 21B, Ipsilateral). In the case of hsp110^{-/-} mice 75% of the mice exhibited significant sensitivity to TBI and increased edema.

### Example 4

### Hsps and Tau Phosphorylation

FIG. 22 (modified from Polier et al., 2008 Cell 133:1068-1079) shows a model for the cooperation of Hsp110 and Hsp70 in protein folding recruitment of Hsp70 to unfolded substrate protein (such as Tau) assisted by Hsp40 (step 1). Formation of complexes between Hsp70 and Hsp110 displaces ADP from the Hsp70 partner (step 2). Direct substrate binding to Hsp110 could provide an anchor aiding the unfolding of kinetically trapped intermediates (e.g in this case tau) through the peptide binding domain (PBD) of Hsp70. Finally, upon binding of ATP to Hsp70, the Hsp70-Hsp110 complexes dissociate and the substrate protein (e.g., tau) is released for folding (step 3). The circle indicates natively folded substrate protein (N). The designations “A”-“E” indicates the possible scenario that p-tau binds to Hsp110 (“A”); and this triggers Hsp110 to bind Hsp70 (“B”); Hsp70/Hsp110/p-tau recruits Pin1 for isomerization and dephosphorylation of p-tau by PP2A (“C”); causing the release of unphosphorylated tau from Hsp110 (“D”); and thereby releasing tau to bind to microtubules (MT) (“E”). This cycle of p-tau isomerization & dephosphorylation requires the activity of Hsp110 and Hsp70.

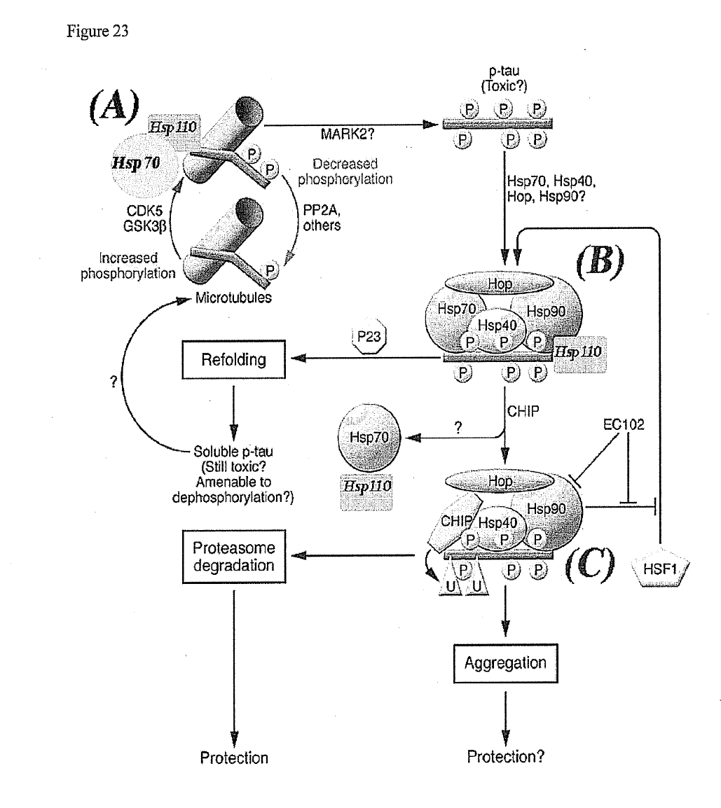

FIG. 23 shows a model for Hsp90 and Hsp70i in suppression of neurotoxicity. Under normal conditions tau protein only contains 2-3 phosphorylation sites and binds microtubules. The p-tau may be the triggering event for the aggregation cascade. p-tau is misfolded and therefore recruits Hsp90 and Hsp70 and co-chaperones to help abnormal p-tau to refold and reincorporate p-tau into microtubules. This reincorporation is contingent upon dephosphorylation of p-tau. It is not known whether Hsp110 is present and forms complexes with Hsp90 and Hsp70. Hsp110/Hsp70i appear to be involved in tau dephosphorylation. Tau may also be transferred to Hsp90 complexes for degradation. Please note that Hsp110 has been added to the model to depict our hypothesis: Panel (A) represents normal physiological pathway for tau phosphorylation/dephosphorylation (as also depicted in FIG. 22) which we believe requires Hsp70/Hsp110. Tau may be transferred to protein complexes in (B) where it is degraded (Lesne et al., 2006 Nature 440:352-357). Under disease conditions, p-tau dephosphorylation and degradation may slow down resulting in accumulation of the complexes presented in C.

As depicted in the models in FIGS. 22 and 23, this example will determine that Hsp110 and Hsp70i bind to tau as a substrate and that this association facilitates tau dephosphorylation through Pin1 and protein phosphatases (e.g., PP2A) and thereby facilitating tau's association with microtubules. We will also test whether Hsp110, Hsp70i, and tau are in complexes with Hsp90, CHIP and their cochaperones which has been presumed to be required for tau (and p-tau) degradation (FIG. 23) (Petrucelli et al., 2004 Hum Mol Genet. 13:703-714; Shimura et al., 2004 J Biol Chem 279:4869-4876). Indeed, Hsp110 is a phosphorylated protein (Ishihara et al., 2003 Biochem J 371:917-925; Ishihara et al., 2000 Biochem Biophys Res Commun 270:927-931) and contains at least four putative pSer/Thr-Pro phosphorylation motifs (Ser385, Ser557, Ser809, and Thr815) (Ishihara et al., 2003 Biochem J 371:917-925; Beausoleil et al., 2006 Nat Biotechnol 24:1285-1292). We hypothesize therefore that, similar to most other Pin1 substrates, Hsp110 pSer/Thr-Pro phosphorylation site (s) is dephosphorylated in a Pin1-dependent manner, and we have designed experiments to identify this phosphorylation site. Once this site is identified, we will determine whether phosphorylation of this site alters the ability of Hsp110 to interact with Pin1 or with tau. Once we identify the phosphorylation site of Hsp110, we will use NMR spectroscopy to confirm whether Pin1 induces cis/trans isomerization of the Hsp110 phosphopeptide containing p-Ser/Thr-Pro. Answering the above questions is critical for designing better strategies to enhance dephosphorylation of p-tau and its increased degradation, thereby decreasing the pathologies observed in tauopathies.

Hsp110 has been confirmed to be a nucleotide exchange factor for Hsp70i (Polier et al., 2008. Cell 133:1068-1079). Indeed hsp70i^{-/-} neurons also exhibit p-tau (FIG. 10). Therefore, in the studies proposed below, we will also analyze the role of Hsp70i in the tau phosphorylation state to determine the impact of Hsp70i deficiency on p-tau. As noted earlier, reports also indicate that Hsp70i interacts with both tau and p-tau (Kosik and Shimura, 2005 Biochim Biophys Acta 1739:298-310). Hsp70i, although stress-inducible in most tissues, it is expressed constitutively in hippocampal neurons as well as in neurons in other regions of the brain and its expression is highly inducible following exposure of the cells to environmental stress (Huang et al., 2001 Mol Cell Biol 21:8575-8591; Zhang et al., 2002 J Cell Biochem 86:376-393).

This example will determine the expression and intra-axonal localization of Hsp110, Hsp70i, tau, and other microtubule components using in vitro neuronal cultures.

Experiment 1. To determine the neuronal expression and intra-axonal localization of Hsp110, Hsp70i, tau, and other microtubule components, primary hippocampal neuronal cultures will be established from wild-type or knockout embryos (Homma et al., 2007 J Neurosci 27:7974-7986). Neurons will then be fixed and immunostained with antibody specific to Hsp110, Hsp70i, Hsc70, Hsp90a and b, a-tubulin, MAP1B, MAP2, total tau, or p-tau (e.g., DA9, AT8, CP13), and the intracellular location of these proteins will be examined using confocal microscopy. Since tau, MAP1B, and tubulin are located in axons, neurons cultured from wild-type or knockout mouse models will be examined to determine the intra-axonal location of the above proteins. We will determine if axonal elongation is affected in neurons from hsp110^{-/-} and hsp70^{-/-} mice compare to wild-type mice by measuring the axonal length and the sizes of small caliber axons (where tau is primarily known to be expressed) using confocal microscopy and image analyses software (Harada and Oguchi, 1994 Nature 369:488-491).

Mice needed. 60 pregnant female mice from +/−x+/−male and female crosses of mouse lines noted above=60 mice.

Statistical considerations. Experiments will be repeated at least 3 times. Data will be expressed as mean+/−SEM. Differences between groups will be analyzed by Student's t test. p values less or equal to 0.05 will be considered significant. In all cases group size will be selected to produce results that are statistically unambiguous.

Interpretation of Experiment 1. If Hsp110 expresses in axons of healthy neurons as tau does, it is conceivable that Hsp110 may be localized with tau in the microtubules and may be involved in dynein and kinesin motor protein transport of cargo along microtubules as reported for tau (Dixit et al., 2008 Science 319:1086-1089). The interaxonal localization of Hsp110 and tau will be confirmed in adult brain tissue sections as well. This is important since neurons cultured from embryos contain p-tau, and therefore the Hsp110 location in relation to tau may be different from that observed in adult neurons.

Methods involved in Experiment 1. Immunoblotting and neuronal cultures prepared from E18 embryos will be as previously reported in our laboratory (Homma et al., 2007 J Neurosci 27:7974-7986; Hu and Mivechi, 2006 Mol Cell Biol. 8:3282-3294; He et al., 1998 Mol Cell Biol 18:6624-6633). Antibodies to Hsp110, Hsp70i, Hsc70, and Hsp90 as well as others will be purchased from Assaydesigns (Ann Arbor Mich.) and StressMarq (Victoria, BC). Antibody to total tau, p-tau, MAP and MAP2 have been obtained from Dr. P. Davies (Albert Einstein College of Medicine) and P. Seubert (Elan Biotech) and commercial sources. The Hsp110^{-/-} and hsp70i^{-/-} mice are in the C57BL/6 genetic background.

This example will also determine if Hsp110 and Hsp70i are substrates of Pin1 and if they directly interacts with Pin1.

Experiment 2. As presented in FIG. 6, Hsp110 interacts with Pin1 and tau in brain extracts prepared from wild-type mice. However, these experiments do not show whether Hsp110 interacts directly with Pin1, or with tau, or that Pin1/Hsp110/tau proteins are in the same or separate complexes. Below, we will perform experiments to determine whether the Hsp110 interaction with Pin1 and tau occurs directly or indirectly. These studies will also show whether phospho-Hsp110 (which is recognized by MPM2 antibody, FIG. 6) requires Pin1 for its isomerization and dephosphorylation by phosphatases (such as PP2A). These experiments will also be performed with Hsp70i, since both Hsp110 and Hsp70i interact with the same substrate as depicted in the model in FIG. 22 (Polier et al., 2008 Cell 133:1068-1079; Barati and Rane, 2006 J Proteome Res 5:1636-1646).

Experiment 2.1. Immunoprecipitation assays will determine if Hsp110 or Hsp70i are a substrate for Pin1 and can be dephosphorylated by PP2A phosphatase using brain extracts. Hsp110 (or Hsp70i) and Pin1 complexes will be immunoprecipitated from brain extracts of wild-type mice using antibody to Hsp110 (or to Hsp70i) (as presented in FIG. 11), and immunoprecipitated materials will be left untreated or treated with purified PP2A, or with CIP (a nonspecific phosphatase). The immunoprecipitated Hsp110 or Hsp70i will then be rinsed with RIPA buffer and examined by immunoblotting using antibody to Pin1. If Pin1 is detected in the original immunoprecipitates, but not following PP2A or CIP treatment, the result will indicate that phosphorylated Hsp110 (or Hsp70i) interacts with Pin1, and that Hsp110 (or Hsp70i) dephosphorylation may require Pin1 (Zhou et al., 2000 Mol Cell 6:873-883). These experiments will be performed with brain extracts obtained from young (5-7 weeks old) or aged (1-1½ year old)+/+, hsp110^{-/-} and hsp70i^{-/-} mice using appropriate antibodies to determine whether there are differences in the quantity of Pin1 interaction with Hsp110 or Hsp70i when one of the proteins is absent. Above experiments will show whether Pin1 interacts with Hsp110 or Hsp70i.

Experiment 2.2. Biochemical assays will determine if Hsp110 or Hsp70i peptides or phosphopeptides containing p-Ser/Thr-Pro are substrates for Pin1. Hsp110 protein possesses four putative Ser/Thr-Pro phosphorylation sites (Ser385, Ser557, Ser809, and Thr815). Furthermore, using MPM2 antibody, we presented data (FIG. 6) that Hsp110 immunoprecipitated from wild-type brain extracts exhibits positive immunoreactivity. To determine whether Pin1 can interact with one or more of the Hsp110 phosphopeptides, experiments will be performed using biotin-labeled unphosphorylated or phosphorylated peptides (21 amino acids) containing Ser385, Ser557, Ser809, or Thr815 residues. We will determine if the biotin-peptides or biotin-phospho-peptides of Hsp110 can interact with Pin1 using ELISA assays by detecting the biotin-labeled peptides binding to the surface-bound purified Pin1 (Zhou et al., 2000 Mol Cell 6:873-883) Biotinylated Hsp110 peptides and phosphopeptides will be purchased from ANASpec, CA. In each case, a group of immunoprecipitates will be treated with PP2A, rinsed with RIPA buffer, and used in immunoblotting experiments (Zhou et al., 2000 Mol Cell 6:873-883). If phospho-Hsp110 binds Pin1, PP2A treatment will release them from each other, and the washing step will remove Pin1 from the complex. Experiments similar to those described in FIGS. 6 and 7 will also be performed to determine and confirm the previous observation that Hsp70i is phosphorylated and may possess MPM2 recognition sites (Barati and Rane, 2006 J Proteome Res 5:1636-1646). Comparable experiments described for Hsp110 will also be performed for Hsp70i if MPM2 antibody can recognize immunoprecipitated Hsp70i and if a direct interaction of these proteins with Pin1 is identified.

Experiment 2.3. Mutational analyses will determine if the Hsp110 phosphorylation site (containing the pSer/Thr-Proline motif) is required for its interaction with Pin1. We will then attempt to identify the Hsp110 phosphorylation site that is important for its interaction with Pin1 by mutating each putative site(s) using site-directed mutagenesis (Hu and Mivechi, 2006 Mol Cell Biol 8:3282-3294). We will mix purified GST-Hsp110 wild-type or individual mutant protein with brain extracts from wild-type, or hsp110^{-/-} mice and pull-down Pin1. Immunoblotting experiments will be used to detect Pin1. Failure to pull-down Pin1 using the GST-Hsp110 mutant will suggest that the mutated amino-acid residue may be the Pin1 binding site. Alternatively, the pull down GST-Hsp110 will be used in immunoblotting experiments using MPM2 antibody. A GST-Hsp110 mutant that does not pull down Pin1 successfully should also lack a MPM2 recognition site. Once the Pin1 binding site on Hsp110 is identified, the GST-Hsp110 wild-type or mutant (and Flag-Pin1) will be coexpressed in N18 neuroblastoma cells, and Pin1 binding will be assessed by immunoblotting to detect Pin1. Alternatively, the complexes of Hsp110 and Pin1 will either be untreated, or treated with PP2A, or CIP, rinsed with RIPA buffer and immunoblotting experiments will be performed to detect Pin1. If Pin1 can no longer be detected to interact with wild-type Hsp110 after PP2A (or CIP) treatment, this will indicate that Pin1 binds the phosphorylated form of Hsp110 in that specific phosphorylation site. Similar experiments described for Hsp110 will also be performed for Hsp70i if we find an interaction of Hsp70i with Pin1. Since no specific substrate for Hsp110 has been identified and we have found that tau binds Hsp110, we anticipate the data obtained from the above experiments will reveal an important role for Hsp110 and Hsp70i in substrate interaction and modification.

Experiment 2.4. Nuclear Magnetic Resonance (NMR) studies will confirm whether Pin1 can isomerize Hsp110 phosphopeptides. A synthetic 21 amino-acid phosphopeptide of Hsp110 containing the pSer/Thr-Pro motif (identified from Experiment 2.3) and purified Pin1 will be mixed and used in NMR studies to confirm whether Pin1 can isomerize the Hsp110 phosphopeptide. Reactions with the Hsp110 phosphorylation mutant and the mutant of Pin1^{K63A} that lack isomerase activity will be used as controls. GST-Pin1 and GST-Pin1^{K63A} proteins will be purified as described previously (Pastorino et al., 2006 Nature 440:528-534; Hu and Mivechi, 2006 Mol Cell Biol 8:3282-3294). This method has been successfully used to show isomerization of Ab phosphopeptides that were found to be a substrate of Pin1 (Pastorino et al., 2006 Nature 440:528-534). NMR studies will be performed at the University of Georgia NMR core facility on a fee-for-service basis. The data analyses and the interpretation of the data will be performed by the Core Facility staff. To test the model depicted in FIG. 22 hypothesizing that Pin1 isomerization of its substrates tau, Hsp110, or Hsp70i may occur, we will pursue comparable experiments as above for Hsp70i. The above experiments will test the model in FIG. 22 (and FIG. 23A) showing that Pin1 isomerizes tau (and perhaps Hsp110 and Hsp70i) leading to tau's dephosphorylation by PP2A.

Interpretation of Experiment 2. Above will determine whether Hsp110 (or Hsp70i) is a substrate of Pin1, and if Pin1 isomerizes Hsp110, facilitating its degradation by a phosphatase (e.g., PP2A) (FIG. 22). Future directions could determine the molecular mechanism of Pin1 regulation of tau and Hsp110 (or Hsp70i) and other microtubule components such as a-tubulin, which has been shown to also interact with Hsp110. In the experiment related to protein phosphatase that dephosphorylate Hsp110 in a Pin1-dependent manner, we will mainly focus on PP2A because this Ser/Thr phosphatase can dephosphorylate substrates following cis-trans isomerization by Pin1 (Galas and Dourlen, 2006 J Biol Chem 281:19296-19304). However, activity of other Ser/Thr phosphatases such as PP1, PP2B, and PP2C may also be considered if appropriate. If we discover Pin1 isomerizes Hsp110 (or Hsp70i), this will undoubtedly opens new avenues of research in the molecular chaperone field in terms of how they impact neurodegenerative diseases.

Methods involved in Experiment 2. Techniques are as previously performed. These methods include molecular cloning, transient transfection assays (Ishihara et al., 2003 Biochem J 371:917-925; Hu and Mivechi, 2006 Mol Cell Biol 8:3282-3294; Inouye and Izu, 2004 J Biol Chem 279:38701-38709), site-directed mutagenesis (Hu and Mivechi, 2006 Mol Cell Biol 8:3282-3294), protein purification using GST-tagged proteins (Hu and Mivechi, 2003 J Biol Chem 278:17299-17306; Keshwani et al., 2008 Protein Expr Purif 58:32-41), and co-immunoprecipitation (Hu and Mivechi, 2006 Mol Cell Biol 8:3282-3294). Mouse N18 neuroblastoma cells, wild-type tau expression vectors have been purchased from ATCC. We will use human Hsp110 or Hsp70i cDNAs in most experiments using expression analyses, since tau and Pin1 cDNAs are of human origin. Human and mouse Hsp110 and Hsp70i possess high levels of sequence conservation, and the phosphorylation sites noted above are conserved between the two organisms. Briefly, for NMR spectroscopy experiments, pSer/Thr-Pro peptides of Hsp110 will be synthesized, purified, and the peptide will be dissolved in buffer (10 mM HEPES, 10 mM NaCl, 10 mM DTT, 5 mM NaN₃, 7% H₂O, pH 7.0) and will either be used directly or mixed with purified Pin1, GST-Pin1, or Pin1 mutant (K63A) at a molar ratio of 60:1 (3 mM pSer-Thr-Pro and 0.05 mM Pin1 or its mutant (Pastorino et al., 2006 Nature 440:528-534)). All experiments will be analyzed at 25° C. on a Varian Inova 800 MHz spectrometer. Analyses will be performed using SigmaPlot (Systat Software, Inc.) by staffs at the University of Georgia NMR Core facility.

Statistical considerations. All experiments will be performed three times. Data will be expressed as mean+/−SEM. Differences between groups will be analyzed by ANOVA or Student's t test as appropriate. p values less or equal to 0.05 will be considered significant. In all experiments, advise of Biostatisticians in the VA or MCG (function as a Core in a fee-for-service basis) will be sought to avoid errors in statistical analyses.

This example will also determine if the Hsp110 or Hsp70i directly interacts with tau, or with p-tau, or if Hsp110, Hsp70i, tau, and Pin1 are in the same complexes and facilitate dephosphorylation of p-tau, or degradation of tau. From the data presented in FIG. 6, it is clear that Hsp110 interacts with tau in brain extracts of wild-type mice and when purified proteins are mixed in vitro. However, it is not known whether tau also interacts with both Hsp70i and Hsp110, or whether any of these proteins must be phosphorylated for this interaction to take place. This information is important to obtain since a direct interaction of Hsp110 or Hsp70i with tau would indicate that these Hsps may be important for tau modification, solubility, or phosphorylation. Since phosphorylated tau is excluded from the microtubules, determining whether the unphosphorylated or phosphorylated forms of tau, Hsp110, or Hsp70i can interact would, at least in part, clarify the role of these proteins and their association with cytoskeletal structure. It would also clarify whether perhaps Pin1 is in the same complexes with Hsp110 and tau proteins and may facilitate dephosphorylation of these two proteins simultaneously.

Experiment 3.1 will use an in vitro purified system to determine the interaction of tau with Hsp70i. We have shown that tau and Hsp110 directly interact in vitro (FIG. 12). To determine whether Hsp70i and tau or Hsp70i, tau and Hsp110 also interact, equimolar concentrations (20 mM) of bacterially purified His-Hsp110 will be mixed with purified tau (tau23, tau40, rPeptides, Inc) in the presence, or absence of GST-Hsp70i. Hsp110 will be immunoprecipitated from the mixture using antibody to histidine, and the complex will be analyzed by immunoblotting to detect tau or GST-Hsp70. This will show whether unphosphorylated form of Hsp110, Hsp70i or tau interact in vitro. Similar experiments will also be performed, but with purified His-Hsp110 expressed in SF9 insect cells, which can support phosphorylation of the Hsp110 protein. The above experiments will test the model in FIG. 22 that tau interacts with Hsp70 and Hsp110 directly. This model predicts that Hsp110 holds the substrate (e.g., tau) while Hsp70 hold the substrate and modifies it.

In Experiment 3.2, immunoprecipitation experiments will determine if GST-Hsp110 is able to pull-down tau or p-tau. The Experiment designed below will test whether Hsp110 is in complexes with p-tau and those including the Hsp70 and Hsp90 that assist with the degradation of tau (FIG. 23) (and under disease conditions, p-tau). Bacterially produced wild-type GST-Hsp110 will be used to pull down tau (or p-tau) from brain extracts of 12-month-old wild-type, or hsp110^{-/-} (or hsp70i^{-/-}) mice as described previously (Hu and Mivechi, 2006 Mol Cell Biol 8:3282-3294). The presence of tau, or p-tau (and Hsp110, Hsp70i, Hsc70, Hsp40, Hsp90, CHIP, HOP, or P23) will be detected by immunoblotting using antibody to total tau or to p-tau (total tau, PFIF1, CP13, and other phospho-specific tau antibodies) and the Hsps noted above. In addition, to determine whether Hsp110 interacts with p-tau, GST-Hsp110-tau (or p-tau) pulled-down fractions will be untreated, or treated with CIP, or PP2A, and rinsed with RIPA buffer to remove unbound protein. GST-Hsp110 (and tau or p-tau) will then be used in immunoblotting experiments to detect tau using total tau or p-tau antibodies. It is expected that if p-tau is bound to phosphorylated Hsp110, then CIP, or PP2A treatment will release tau from GST-Hsp110, and the immunoblots of phosphatase-treated samples will be negative for the presence of tau. Alternatively, if tau is detected to remain bound to GST-Hsp110 following CIP or PP2A treatment, then tau will be detected in immunoblots of phosphatase-treated samples. As a control, additional comparable experiments will be performed using GST-Hsp70i to determine whether this protein can pull-down tau or p-tau. Previous data suggest that Hsp70i interacts with both tau and p-tau (Shimura et al., 2004 J Biol Chem 279:4869-4876).

Interpretation of Experiment 3. The studies described in Experiment 3 will reveal whether Hsp110 or Hsp70i directly interact with tau (or p-tau); this can easily be accomplished using the purified system. The studies will also show whether Hsp110 can pull down tau or p-tau from brain extracts of wild-type or hsp110^{-/-} (or hsp70^{-/-}) mice that contain high levels of p-tau. We do not anticipate any problems performing these experiments. Consideration will also be given to the fact that Hsp110, Hsp70i, and tau may both have to be phosphorylated for them to interact. The brain extracts of young and old (containing more p-tau for knockout mice) wild-type, hsp110^{-/-} or hsp70i^{-/-}, will strengthen the outcome of the experiments to show a specific interaction of tau or p-tau with Hsp110 or Hsp70i. The experiment will also detect Hsp90 and CHIP ubiquitin ligase that has been shown in the p-tau complexes suggesting CHIP may be involved in tau (or p-tau) degradation.

Methods involved in Experiment 3. Isolation of GST fusion proteins and GST-pull down techniques are as previously described. Plasmids pBIEx (EMD biotechnology) will be used for transfection of cDNAs (such as Hsp110) into SF9 cells and subsequent protein purification from SF9 insect cells (Novagen).

Mice needed. brain extracts of wild-type, hsp110^{-/-}, or hsp70i^{-/-} mice will be used when required. 10 mice/group×3 groups=30 mice.

Statistical considerations. All experiments will be performed three times. Data will be expressed as mean+/−SEM. Differences between groups will be analyzed by Student's t test. p values less or equal to 0.05 will be considered significant. In all cases group size will be selected to produce results that are statistically unambiguous.

Experiment 4 will determine whether Hsp110, Hsp70i, tau, or Pin1 are in the same complexes using a sequential immunoprecipitation procedure. His-Hsp110, HA-Hsp70i, tau, or Flag-Pin1 will be cotransfected into hsp110^{-/-} (or hsp70^{-/-}) MEFs and expression of individual protein will be detected in cell lysates to ensure transient transfections have been successful. 48 hrs after cotransfection of expression plasmids, tau will be immunoprecipitated from 1 mg of cotransfected cell lysates and (Hu and Mivechi, 2006 Mol Cell Biol 8:3282-3294) the presence of Hsp110, Hsp70i, Pin1, and other proteins such as Hsp90, Hsp40, CHIP, HOP, and p23 protein in the immunoprecipitated samples will be confirmed by immunoblotting. Then, an immunoprecipitation experiment will be performed using the protein complexes from the first immunoprecipitation step, but in this case instead of using antibody to tau, antibody to Flag-Pin1 will be used to pull-down Hsp110 and tau. The presence of Hsp110 and tau and the other components noted above will be confirmed by immunoblotting. If Hsp110, Hsp70i, Pin1, tau, and Hsp90, CHIP are detected in the second immunoprecipitation step, this will indicate that Pin1, tau, and Hsp110 are in the same complexes with Hsp90, CHIP and others. If on the other hand, Flag-Pin1 (or HA-Hsp70i) can only immunoprecipitate Hsp110 and tau but not others (e.g., Hsp90, CHIP), this will indicate that in the first immunoprecipitation step, the antibody to tau brought down Pin1 or Hsp110 or other components, in separate complexes. Sequential immunoprecipitation experiments will be as described by Hu and Mivechi (2006. Mol Cell Biol 8:3282-3294).

Interpretation of Experiment 4. Determining whether the Hsp110/Hsp70i/Pin1/tau complexes also contain Hsp90, HOP, P23 and CHIP ubiquitin ligase will determine whether dephosphorylation of tau by Pin1 and PP2A (with the help of Hsp70/Hsp110) is tied in with tau degradation via Hsp90/CHIP complexes. Future directions could include determining whether Hsp110 or Hsp70i facilitate p-tau dephosphorylation under physiological or disease conditions so that mechanisms can be exploited to find drugs to ensure dephosphorylation or degradation of p-tau. Methods involved in Experiment 4. Transient transfection assays, immunoblotting, and sequential immunoprecipitation techniques have previously been performed in our laboratory (Hu and Mivechi, 2006 Mol Cell Biol 8:3282-3294). MEFs has already been prepared from wild-type and hsp110^{-/-} or hsp70i^{-/-} E13.5 embryos.

Statistical considerations. All experiments will be performed three times. Data will be expressed as mean+/−SEM. Differences between groups will be analyzed by Student's t test. p values less or equal to 0.05 will be considered significant.

This example will also determine whether the presence of Hsp110, Hsp70i, and their interaction with Pin1 and/or tau are required for stability of microtubules using biochemical assays.

In Experiment 5, biochemical and immunoblotting assays will detect the presence of p-tau in the soluble and insoluble brain extracts, and to determine whether p-tau in hsp110^{-/-} brain extracts is associated with microtubule fractions. Brain extracts of cohorts of wild-type or hsp110^{-/-} mice at 2-4, 6-8, and 12-17 months of age will be extracted, and soluble, Sarkosyl-soluble, and Sarkosyl-insoluble (pellet) fractions will be prepared by the methods described previously (Scheme 1 below) (Dou et al., 2003 PNAS 100:721-726; also see FIG. 4). Immunoblotting experiments of different fractions (Scheme 1; supernatant 1 and 2, and pellets 1 and 2) will be performed using antibodies to detect total tau, p-tau (CP13, 12E8, PHF1, AT180, AT8), or Alzheimer's type conformation of tau (Alz50 or MC1). Immunoblotting experiments of p-tau show that p-tau exhibits slower mobility on SDS-PAGE. In order to determine if other Hsps fractionate with p-tau and/or Hsp110, we will determine the levels of Hsp110, Hsp90, Hsp70, Hsc70, CHIP (Goryunov and Liem, 2007 J Clin Invest 17:590-592; Petrucelli et al., 2004 Hum Mol Genet. 13:703-714; Dickey et al., 2007 J Clin Invest 117:648-658), Pin1, MAP1 (and MAP1B), α and β tubulin, α-synuclein (as a control), Aβ precursor protein, and GAPDH by immunoblotting (Dickey et al., 2006 J Neurosci 26:6985-6996). Hsps (e.g., Hsp90 and Hsp70; see FIG. 23) have been found in p-tau-containing fractions. The presence of Hsp110 in different fractions with or without tau has not been elucidated.

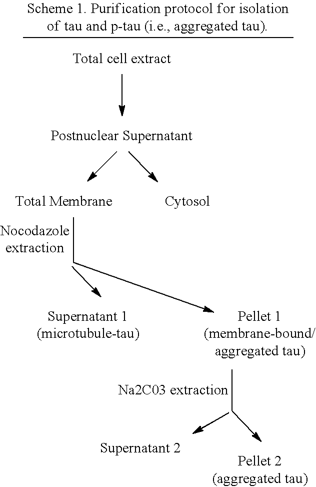

To determine the extent of pathology in different brain regions, we will use cortex, hippocampus, medulla, and cerebellum for the above analyses. In addition, to ensure the presence of p-tau, Sarkosyl-insoluble extracts (pellet) will be treated with phosphatase, and immunoblotting experiments will be performed to ensure the disappearance of high molecular weight p-tau (Liou et al., 2003 Nature 424:556-561). AT8 (eBioscience) and PHF1 antibodies recognize the slower migrating 68 kDa p-tau protein, which is also detectable by Alz50 and MC1 antibodies, that detect NFT-specific conformations. AT180 recognizes pThr231, which is recognized by Pin1. Above experiments will reveal whether the amount of proteins noted above in the insoluble fraction increases with age in hsp110^{-/-} brain extracts compared to wild-type.

Statistical considerations. All experiments will be performed with brain extracts from 10 mice/group of different age groups. Data will be quantitated using image analysis software and expressed as mean+/−SEM. Differences between groups will be analyzed by Student's t test. p values less or equal to 0.05 will be considered significant. In all cases group sizes will be selected to produce results that are statistically unambiguous.

Mice needed. 10 mice×3 age groups×2 groups (wild-type, hsp110^{-/-})=60 mice.

Interpretation, potential problems, and future directions of Experiment 5. The results of the above experiments will show whether Hsp110 interacts with p-tau and is excluded from the microtubules, as has been shown previously for Hsp70 (Dou et al., 2003. PNAS 100:721-726). This experiment will also indicate that Hsp110 may be present in complexes as depicted in FIG. 23. The presence of Hsp110 in such complexes has not previously been investigated. Our findings show for the first time, that Hsp110 could regulate the tau phosphorylation state and perhaps its degradation under physiological conditions (FIG. 22). The p-tau, Hsp70i, Hsp90, HOP, and CHIP have been detected together with NFTs due to the fact that they are important components of the tau dephosphorylation and degradation pathway (Dickey et al., 2007 J Clin Invest 117:648-658). Accumulation of these proteins in the Alzheimer's brain together with Aβ could only suggest their important roles in p-tau dephosphorylation and degradation pathway. Future directions could include the generation of an hsp110^{-/-}hsp70i^{-/-} mouse line which will be powerful to examine the role of these two proteins in the tau phosphorylation and degradation pathway. In addition, future direction could include confocal microscopy to determine the impact of Pin1 isomerization of Hsp110 on intracellular location of Hsp110 and its interaction with tau or α-tubulin.

Methods involved in Experiment 5. Above experiments will be performed as described previously (Goryunov and Liem, 2007 J Clin Invest 17:590-592; Dou et al., 2003 PNAS 100:721-726; Petrucelli et al., 2004 Hum Mol Genet. 13:703-714; Dickey et al., 2007 J Clin Invest 117:648-658).

Statistical considerations. All experiments will be performed three times. Data will be quantitated using image analysis software and expressed as mean+/−SEM. Differences between groups will be analyzed by Student's t test. p values less or equal to 0.05 will be considered significant. In all cases group sizes will be selected to produce results that are statistically unambiguous.

This example will also determine the pathology, expression, APP processing and Aβ production, and behavioral pattern using our in vivo mouse models. Immunofluorescent studies presented in Example 1 (FIGS. 3 and 11) show increased appearance of p-tau in hsp110^{-/-} and hsp70i^{-/-} brain tissue sections in aging mice. However, additional studies are required to deter nine the extent of p-tau present in the brain of hsp110^{-/-} or hsp70i^{-/-} mice that are 15-18 months of age and their contribution in APP processing in vivo. These experiments will reveal more accurately the participation of these genes in tau pathology.

In Experiment 6.1, immunohistochemical staining and biochemical analyses will detect p-tau, APP processing in young and aged wild-type mice and hsp110^{-/-} and hsp70i^{-/-} mice. We have performed immunohistochemical staining of p-tau in hsp110^{-/-} mice at different age groups using specific antibodies and have briefly analyzed the hsp70i^{-/-} mice as presented in FIGS. 4 and 12. However, we have not competed comparative analyses with older wild-type, hsp110^{-/-}, or hsp70i^{-/-} mice (e.g., 12-17 months of age) using specific p-tau antibodies (e.g., monoclonal antibody AT180, which recognizes p-tau at Thr231) which is known to undergo isomerization by Pin1 (Liou et al., 2003 Nature 424:556-561). To accomplish these experiments brain tissue sections of these mice will be analyzed and number of cells expressing total tau or those expressing p-tau using antibody to p-tau (12E8 detects S262/356; CP13 detects S202/T205; PHF1 detects S395/S404; and AT180 detects Thr231) will be determined (Liou et al., 2003 Nature 424:556-561). To determine the expression pattern of APP, immunohistochemical staining will also be performed with anti-mouse antibody to APP (Upstate Biotech.), which recognizes amino acid residues 66 to 81 of the N terminus. This antibody recognizes all isoforms of murine APP (Wen et al., 2008 J Neurosci 28:2624-2632). In addition, APP processing and Aβ production will be examined in the 2, 5-6, and 12-17-months-old wild-type, hsp110^{-/-}, and hsp701 mice. APP is cleaved by β-secratase (BACE) to generate C99 (also known as β-CTF) from Carboxyl Terminal Fragment (CTF), and a secreted fragment, sAPPβ is released into the extracellular space. APP can also be released by α-secretase to generate C83 (also known as α-CTF) and sAPPα which precludes n-processing (Wen et al., 2008 J Neurosci 28:2624-2632; Schneider and Rajendran, 2008 J Neurosci 28:2874-2882). A significant increase in p processing would correlate with a significant increase in Aβ production. Aβ production will be determined in brain extracts using ELISA (Wen et al., 2008 J Neurosci 28:2624-2632). Soluble murine Aβ1-40 and Aβ1-42 from a region including cortex and hippocampus will be determined using antibodies Aβx-40 (human) and Aβx-42 (JRF cAβ42/26) and JRF/AβN-25 (human) IRF/rAβ1-15/2 for detection (R&D Inc., Signet Labs, PA) as previously described (Schmidt et al., 2005 Methods Mol Biol 299:279-297). Tissue preparation will be performed as previously described (Schmidt et al., 2005 Methods Mol Biol 299:279-297). The above experiments will examine the extent of p-tau present in wild-type, hsp110, or hsp70i-deficient mice. In addition, these studies will show whether APP expression and processing are altered in mutant mice compared to wild-type mice.

Statistical considerations. All experiments will be performed with brains from 7 mice/group of different age groups. Data will be quantitated and expressed as mean+/−SEM. Differences between groups will be analyzed by Student's t test or ANOVA (for multiple group comparisons). p values less or equal to 0.05 will be considered significant. In all cases group sizes will be selected to produce results that are statistically unambiguous.

Mice needed. 7 mice×3 age groups×3 mouse lines (wild-type, hsp110^{-/-}, hsp70^{-/-})=63.

In Experiment 6.2, immunohistochemical staining will detect apoptotic cells and activated microglia in wild-type, hsp110^{-/-}, and hsp70i^{-/-} brain tissue sections. Both apoptotic cells and activated microglia have been observed in AD brain tissue sections containing p-tau and amyloid deposits (Lewis et al., 2001 Science 293:1487-1491; Williams, 2006 Intern Med J 36:652-660). Brain tissue sections of wild-type and knockout mouse lines at 2-4, 6-8, and 12-17 months of age will be immunostained with Mac3 (activated microglia), and immunopositive cells will be quantitated. Similarly, to detect an inflammatory response, brain sections of above mouse lines will be immunostained with anti-CD11b antibody, and the number of positive cells will be quantitated. Since NFTs have been associated with astrogliosis, we will immunostain and qunatitate the number of astrocytes (Glial Fibrillary Acidic Protein (GFAP) positive) in such sections (Liou et al., 2003 Nature 424:556-561). The brain tissue sections will be analyzed by TUNEL assay, and the number of apoptotic cells will be quantitated (Homma et al., 2007 J Neurosci 27:7974-7986). The above experiments will compare the pathology associated with expression of p-tau in wild-type and knockout mice.

Statistical considerations will be as described in Experiment 6.1. For each immunostaining experiment, at least 20 sections of the same region of the brain (e.g., hippocampus) per mouse from 7 mice of the same age group will be analyzed.

Mice needed. No additional mice are needed. Brain tissue sections will come from mice used in Experiment 6.1.

In Experiment 6.3, histochemical staining of brain tissue sections will detect NFTs in wild-type, hsp110^{-/-}, or hsp70i^{-/-} mice. To more extensively examine the NFTs, we will immunostain section of different brain regions of wild-type, hsp110^{-/-}, and hsp70i^{-/-} mice at 2-4, 6-8 and 12-17 months of age using Congo red, Thioflavin S, or Bielschowsky staining (Liou et al., 2003 Nature 424:556-561; Roberson et al., 2007 Science 316:750-754). We will quantitate the number of cells containing NFTs in different brain regions. The above tests will show the extent of NFTs in young and aged wild-type and mutant mice.

Statistical considerations will be as described in Experiment 6.1.

Mice needed. No additional mice are needed. Brain tissue sections will come from mice used in Experiment 6.1.

Experiment 6.4 will detect protein kinase and phosphatase activities in wild-type, hsp110^{-/-}, and hsp70i^{-/-} brain extracts. The appearance of p-tau has been associated with an increase in the relevant protein kinase activity or decrease in protein phosphatase (e.g., PP2A) activity (Ballatore et al., 2007 Nat Rev Neurosci 8:663-672; Liou et al., 2003 Nature 424:556-561). To examine the mechanism that induces phosphorylation of tau epitopes, we will compare the activity of protein kinases and phosphatases in brain extracts from different regions of the brain of wild-type and knockout mouse lines at 1-4, 6-8 and 12-17 months of age. The activities of the protein kinases that will be tested will be those presumed to phosphorylate tau, such as cyclin-dependent kinase (CDK) or glycogen synthase kinase 3β (GSK3β) (Liou et al., 2003 Nature 424:556-561; Noble and Olm, 2003 Neuron 38:555-565). The activity of Ser/Thr phosphatases such as PP2A towards motifs in p-tau will be examined (Liou et al., 2003 Nature 424:556-561; Wang et al., 2007 Eur J Neurosci 25:59-68). The kinase and phosphatase activities will be measured in the total brain extracts before, and also after, the pathological problems are observed in hsp110^{-/-} or hsp70i^{-/-} mice (Liou et al., 2003 Nature 424:556-561).

Statistical considerations. All experiments will be performed with brain extracts from 7 mice/group of different age groups. Data will be quantitated and expressed as mean+/−SEM. Differences between groups will be analyzed by Student's t test. p values less or equal to 0.05 will be considered significant. In all cases, group size will be selected to produce results that are statistically unambiguous.

Mice needed. 7 mice per group×3 age groups (1-4-, 6-8-weeks and 12-17 months×3 mouse lines (wild-type, hsp110^{-/-}, hsp70i^{-/-})=63 mice.

Experiment 6.5 will determine the behavioral impairments in hsp110^{-/-} and hsp70i^{-/-} mice. Accumulation of p-tau and neuronal death may associate with behavioral impairment (Liou et al., 2003 Nature 424:556-561). To determine whether mice deficient in hsp110 or hsp70 exhibit behavioral deficits, we have begun to analyze their behavior using multiple tests. We have tested hsp110^{-/-} mice in Open Field Locomotor Activity compared to wild-type mice. Our results show that hsp110^{-/-} mice exhibit normal locomotor activity at one year of age. However, as we presented earlier, 12-months-old hsp110^{-/-} mice exhibit signs of impairment in the Contextual Fear Conditioning test (FIG. 11) (Lalonde, 2002 Neurosci Biobehav Rev 26:91-104). These experiments have not been performed with hsp70i^{-/-} mice.

To determine the long-term impact on behavior of hsp70i (or hsp110, e.g., Rotarod) gene deficiency, we will perform Open Field Locomotor activity, Y-Maze/Spontaneous Alternation, Rotarod, and Two Trial Recognition memory tests. These tests assess the normal navigation behavior of rodents as follows. In Open Field tests, various components of horizontal, vertical and stereotyped behavior can be determined. In Rotarod tests, an animal's ability to remain on a rotating rod as the speed of rotation increases will be determined. This is sensitive to damage in basal ganglia and cerebellum. Success in the Y-Maze/Spontaneous Alternation test is indicated by high rate of alternation in control groups indicating that the animals can remember which arm was entered last. Each of the Y-maze tests is sensitive to hippocampal damage (Lalonde, 2002 Neurosci Biobehav Rev 26:91-104). The Fear Conditioning tests determines Context-dependent Freezing and Cue-dependent Freezing. These tests evaluate the learned aversion of an animal for an environment that has been associated with a negative stimulus, usually mild shock. The animal will be placed in a novel environment (i.e., with different lighting, olfactory cues, and visual cues), and freezing behavior associated with the tone is measured.

Statistical considerations. For behavioral studies at least 12 male mice/group of two different age groups will be used. Data will be quantitated and expressed as mean+/−SEM. Differences between groups will be analyzed by Student's t test. p values less or equal to 0.05 will be considered significant. In all cases, group size will be selected to produce results that are statistically unambiguous.

Mice needed. 12 mice per group×2 age groups (6-7 months of age and 12-17 months)×3 mouse lines (wild-type, hsp110^{-/-}, hsp70i^{-/-)=}72 mice.

Interpretation of Experiment 6. The above experiments will show the pathology observed in hsp110^{-/-} and hsp70^{-/-} mice compared to wild-type mice. Hsp110^{-/-} mice have been under observation in our laboratory for about 18 months and hsp70i^{-/-} mice for a longer time period but we have not been extensively analyzed them in teens of brain pathology. The above experiments will determine whether the activity of protein kinases and phosphatase increase or decrease with age, respectively. An increase in GSK3b or Cdk5 activity or decrease in PP2A activity has been associated with increased levels of p-tau in different models of tauopathies (Liou et al., 2003 Nature 424:556-561; Galas and Dourlen, 2006 J Biol Chem 281:19296-19304; Michel and Mercken, 1998 Biochim Biophy Acta 1380:177-182). Behavioral tests will determine the role of Hsp110 and Hsp70i in brain pathology. As noted in FIG. 22, Hsp110 performs its function at least in part in association with Hsp70i. Therefore, inclusion of Hsp70 in appropriate experiments as indicated above is important to determine whether Hsp70i's role in tau pathology is comparable to Hsp110 or vastly different. If warranted, additional studies will be designed for each knockout mouse model to address their specific role in tau pathology.

Methods involved in Experiment 6. Immunohistochemistry, kinase assays using immunocomplex kinase assays, and phosphatase assays, and TUNEL assay will be as previously described. (Homma et al., 2007 J Neurosci 27:7974-7986; Hu and Mivechi, 2006 Mol Cell Biol 8:3282-3294; He et al., 1998 Mol Cell Biol 18:6624-6633; Michel and Mercken, 1998 Biochim Biophy Acta 1380:177-182; Mivechi and Giaccia, 1995 Cancer Res 55:5512-5519; Mivechi et al., 1994 J Cell Biochem 54:186-197). Antibodies to Hsps or to total tau and p-tau will be as described in Experiment 1, Method Section. Cell extracts preparation, APP processing and Ab production using ELISA assay will be as described in Experiment 7 and by others (Lewis et al., 2001 Science 293:1487-1491; Lesne et al., 2006 Nature 440:352-357; Wen et al., 2008 J Neurosci 28:2624-2632; Schneider and Rajendran, 2008 J Neurosci 28:2874-2882). Behavioral studies will be performed in the Small Animal Behavioral Core Facility in MCG (Ma et al., 2007 Neuroscience 147:1059-1065; Dineley et al., 2002 J Biol Chem 277:22768-22780). All mouse lines are in the C57BL/6 genetic background. Use of males and females will be equally considered in each experiment.

### Example 5

Hsps and Aβ Production

As an example of neurodegenerative diseases associated with tauopathy, we will cross hsp110^{-/-} mice with an existing mouse model expressing variants of Aβ APP (Tg2576⁺) to establish the role of Hsp110 in APP processing and Aβ production. Data suggests that reduction of mutant tau or normal tau levels in the brain can alleviate memory loss and other neurological shortcomings to reproduce human Alzheimer's pathology (Marx, 2007 Science 316:1416-1417). Therefore, reducing the levels of tau may lead to complementary anti-amyloid treatment in the clinic. Thus far, from the evidence of Aβ deposition in brains of AD patients, the prevailing thought was to put tau as a downstream event in Alzheimer's pathology (Marx, 2007 Science 316:1416-1417). Data from the literature suggests that Hsp70 affects APP processing in tissue culture cells (Kumar et al., 2007 Hum Mol Genet. 16:848-864). Since hsp110^{-/-} mice exhibit p-tau, NFTs, and positive immunoreactivity using antibody to Alzheimer's conformational tau epitope (MC1 antibody) early in life, we anticipated that we may be able to address whether hsp110^{-/-} mice exhibit accelerated pathology when crossed with transgenic mice carrying a mutant isoform of human APP. Transgenic Tg2576 mice expressing a 695 amino-acid isoform of human APP derived from a large Swedish family with early onset AD is a well-established model for AD (Lesne et al., 2006 Nature 440:352-357). Young Tg2576⁺ transgenic mice (less than 6 months of age) have normal memory and lack neuropathology, middle-aged mice (6-14 months old) develop memory deficits without neuronal loss, and older mice (>14 months old) exhibit numerous neuritic plaques containing p amyloid (Lewis et al., 2001 Science 293:1487-1491; Lesne et al., 2006 Nature 440:352-357). Concomitant with extensive deposition of senile-like plaques, these mice exhibit inflammatory and microglial responses associated with these plaques. As we are very encouraged from the data presented in FIG. 13, to determine whether lack of the hsp110 gene, which leads to development of tau pathology in mice at an early age, is influenced by pathogenic mutations that cause AD, we will more extensively examine brain pathology (neuronal loss, plaque deposition, inflammatory response) in Tg2576⁺ and hsp110^{-/-}Tg2676+ mice. Our findings that Hsp110 controls tau phosphorylation and therefore may be important in maintaining brain homeostasis could be a critical finding, especially if we confirm that hsp110^{-/-}Tg2576⁺ mice exhibit accelerated or increased levels of insoluble 442 (toxic species) production compared to Tg2576⁺ mice. Future directions will include hsp70i^{-/-}Tg2576⁺ mice in the analyses. If the results of hsp110^{-/-} Tg2576⁺ mice reveal critical information of the role of Hsp110 in APP processing, it may be that other Hsps could also play a vital role in APP processing in vivo. Analyses of both mouse models crossed with Tg2576⁺ mice are important since each protein may have their own unique function as well as overlapping function, e.g., Hsp70i is highly induced in the brain following exposure to environmental stress. The proposed experiments in this aim will demonstrate the critical role of Hsp110 in AD pathology.

Experiment 7.1 will determine whether Hsp110 regulates Aβ production in vivo. To determine whether absence of the hsp110 gene leads to the accelerated pathology observed in Tg2576⁺ mice, we will perform histological and immunohistological analyses with wild-type, hsp110^{-/-}, Tg2576⁺, and hsp110^{-/-}Tg2576⁺ mice at 2-4, 6-8, and 12-17 months of age to determine a time course for appearance of p-tau, NFTs, and Aβ in senile plaques. Determination of the p-tau in the above mouse lines will essentially be as those presented in Example 1 and those outlined in Experiment 6. Appearance of Aβ will be determined in brain tissue sections using antibody to Aβ using monoclonal antibody corresponding to amino acid residues 1-11 of human β-amyloid (Sigma). This antibody recognizes human β-amyloid precursor protein (APP), soluble APP and Aβ (1-40/42) (Lesne et al., 2006 Nature 440:352-357). Above experiments will also be performed with hsp70i^{-/-} mice.

In Experiment 7. 2, biochemical assays will determine the soluble and insoluble Aβ production in Tg2576⁺, and hsp110^{-/-}Tg2576⁺ brain extracts. Neuropathological effects of AD are the appearance of NFTs, which include p-tau and neuritic plaques that include Aβ peptides derived from APP (Lewis et al., 2001 Science 293:1487-1491; Lesne et al., 2006 Nature 440:352-357). However, the exact relationship between p-tau and other Alzheimer's pathology is unknown. From the studies proposed earlier, we will determine whether hsp110^{-/-} mice exhibit impaired APP processing and Aβ production, compared to wild-type mice. If the results show that the absence of Hsp110 affects APP processing, crosses of hsp110^{-/-} mice with Tg2576⁺ mice will show the pathological consequences of Hsp110 deficiency in this model in a more significant manner. Since Pin1 has been shown to act on the Thr⁶⁶⁸-Pro motif of APP and regulates APP processing and Aβ production, and since we have found that Hsp110 deficiency leads to accumulation of p-tau and lower Pin1 activity, we hypothesize that these features of hsp110^{-/-} mice may increase insoluble Aβ42 production in hsp110^{-/-}Tg2576⁺ mice when compared to Tg2576^{-/-} mice. However, Hsp110 may directly affect APP processing as has been shown for Hsp70 using cultured cells (Kumar et al., 2007 Hum Mol Genet. 16:848-864).

Therefore, since Tg2576^{-/-} mice exhibit deposition of Aβ oligomers, we will compare a cohort of wild-type, hsp110^{-/-}, Tg2576% and hsp110^{-/-}Tg2576⁺ mice at 2-4, 6-8, or 12-17 months of age in terms of expression level of soluble Aβ and Aβ oligomers in soluble, extracellular-enriched extracts of protein from the brain using immunoblotting with antibody specific to human Aβ (such as 6E10 and 4G8 from Abeam and Sigma Chem. Co). From these immunoblots, the amounts of monomers to docamers and sAPPα (secreted form of APP that has been cleaved by α-secretase) will be quantified. The apparent molecular weights of monomer to docamers are resolved in SDS-PAGE from below 10 kDa to approx. 100 kDa as detected using 6E10 monoclonal antibody that recognizes amino acid 1-17 of human Aβ peptide (Lesne et al., 2006 Nature 440:352-357). The molecular size of sAPPα is about 100 kDa. The appearance of oligomers Aβ 56 kDa has been correlated at 6 months of age with memory loss in Tg2576⁺ mice. We will therefore compare the immunoblots between hsp110^{-/-}Tg2576⁺ and Tg2576⁺ mice of the comparable ages as noted above. Alternatively, brain extracts of wild-type, hsp110^{-/-}, Tg2576⁺, and hsp110^{-/-}Tg2576⁺ littermates will be examined at 2-4, 6-8, and 12-17 months of age for accumulation of Aβ42 in multivesicular bodies of neurons (Roberts et al., 1994 J Neurol Neurosurg Psychiatry 57:419-425). The expression of soluble Aβ40 and Aβ42, and insoluble Aβ42 will be determined in brain extracts using ELISA. These studies will indicate whether hsp110 deficiency causes an age-dependent, selective increase in insoluble Aβ42 levels that is accelerated by APP expression in Tg2576⁺ mice. Above experiments will also be performed with hsp70I^{-/-}, hsp70I^{-/-}Tg2576⁺ and the results will be compared between the two knockout mouse lines. Using hsp70i^{-/-} mice is important since in an in vitro cell culture model Hsp70i has been shown to interact and be involved in APP processing. The impact of Hsp70i deficiency in APP processing in vivo has not been accomplished.

Interpretation of Experiment 7. As noted before, hsp110^{-/-} mice exhibit p-tau and several other phenotypes associated with tauopathies. The studies designed in Experiment 7 will attempt to extend our preliminary observation that Hsp110 (or Hsp70) deficiency impacts development of pathology in the Tg2576⁺ mouse model. We anticipate that hsp110 deficiency will lead to acceleration of pathology in hsp110^{-/-} Tg2576⁺ mice compared to Tg2576⁺ mice and this hypothesis appears to be correct; however, we are uncertain about the contribution of Hsp110 in Aβ generation and the severity of the phenotypes that we may observe. Wild-type and hsp110^{-/-} mice will be used as controls. The density of NFTs will be determined and the distribution of NFTs in different parts of the brain will be examined to determine whether areas of damage are more extensive in the brain of hsp110^{-/-}Tg2576⁺ compared to Tg2576⁺ mice. In addition, the experiments designed above will reveal whether endogenous Aβ forms a ladder of stable, soluble, assemblies and multiples of trimers in Tg2576⁺ and hsp110^{-/-}Tg2576⁺ mice at comparable ages or if hsp110^{-/-}Tg2576⁺ mice will exhibit more rapidly the Aβ oligomers that normally appear at 6 months of age in Tg2576⁺ mice at the onset of memory deficit. In all experiments we will use equal numbers of male and female mice to ensure detection of any differences in pathology that may be observed between the sexes. Results will be compared between Experiments 6.1 and 7 to ensure a conclusion can be made between endogenous murine APP processing and the human mutant APP (in Tg2576+ mice) processing in wild-type or hsp110^{-/-} mice. Crossing hsp110^{-/-} (and hsp70i^{-/-}) with Tg2576⁺ will add novel information about the role of these molecular chaperones in Alzheimer's pathology since a number of studies point to a critical role for Hsps in Alzheimer's pathology, but no direct evidence in vivo has been put forward so far. Future directions could include determination of the expression and activities of Hsp110 and Hsp70 in neurodegenerative disorders using appropriate model systems. In addition, we could test to determine whether transient activation of heat shock factor Hsf1 that increases the levels of Hsps (Huang et al., 2001 Mol Cell Biol 21:8575-8591; Zhang et al., 2002 J Cell Biochem 86:376-393) decrease the progression of AD or other neurodegenerative diseases using mouse models.

Methods involved in Experiment 7. To reliably separate the specific cellular pools of Aβ (such as extracellular, intracellular, membrane-associated, and insoluble) we will use extraction protocols recently developed by Lesne et al., (2006 Nature 440:352-357). Briefly, mouse brains will be homogenized and centrifuged to remove large debris. This will be followed by a second centrifugation at 100 K×g for 40 mm. The pellet will then be suspended in buffer containing 1% Triton X-100 to extract membrane proteins and centrifuged at 100 K×g for 40 min. The resulting supernatant represents the Triton X-100 membrane-extracted fraction. The full-length APP and the C-terminal fragments of APP in the total cell lysates and Triton X-100 membrane-extracted fraction will be determined using C-terminal polyclonal antibodies raised against the APP intracellular domain (Sigma). In the insoluble fraction, the secreted total APPs (αAPPs and βAPPs) will be detected using 22C11 monoclonal antibody (mAb) (Chemicon) raised against the N-terminal domain of APP.

The αAPPs can also be detected using 6E10 mAb raised against amino acid residues 1-17 of human Aβ peptide, and the βAPPs can also be detected using 197sw mAb raised against the Swedish mutant form of APPs. The 197sw mAb will be provided by Elan Pharmaceuticals. After immunoblotting, the levels of mature and immature APP, phosphorylated APP (full length and C-terminal fragments), soluble APP and C-terminal fragments will be detected using chemiluminescence and will be semi-quantified using NIH Image1.63 software. The levels of Aβ40 and Aβ42 will also be determined by sandwich ELISA in one hemisphere of each mouse at different ages, as described previously (Lesne et al., 2006 Nature 440:352-357; Johnson-Wood et al., 1997 PNAS 94:1550-1555; Citron et al., 1997 Nat Med 3:67-72). Briefly, brain tissues will be homogenized in 50 mM NaCl and 0.2% diethanolamine and spin for 45 min at 100 K×g. The supernatant will be neutralized by a 1/10 volume of 0.5 M Tris-HCl, pH 6.8 and will be used as soluble fraction. The remaining pellets will be rinsed with buffer containing 50 mM NaCl and 0.2% diethanolamine followed by addition of formic acid, sonication to disperse the pellet, and centrifugation at 130 K×g for 45 min. The aqueous supernatant will be neutralized with 19-fold volume neutralization buffer (1 M Tris base, 0.5 M Na₂HPO₄, 0.05% NaN₃).

In all assays, the 2G3 and 21F12 antibodies will be used to detect Ab-40 and Ab-42. The biotinylated mAb 266B, against domain 13-28, will be used as the detecting antibody. The reporter system contains streptavidin-alkaline phosphatase, and AttoPhos (Promega) will also be used as the substrate (excitation, 450 nm; emission, 580 nm). The mAbs 2G3, 21F12 and 266B are presently being obtained from Elan Pharmaceuticals. The hsp110^{-/-} and hsp70i^{-/-} mice are in the C57BL/6 genetic background and Tg2576⁺ transgenic mice are in C57BL/6-SJL genetic background and the intercrossing will remain in F2 generation to remain consistent between mouse lines.

Statistical considerations. All experiments will be performed 3 times and with sufficient number of mice to generate results that are statistically unambiguous. Data will be expressed as mean+/−SEM. Differences between groups will be analyzed by Student's t test or ANOVA. p values less or equal to 0.05 will be considered significant.

Mice needed for Experiments 7.1 and 7.2. 10 mice each×6 groups (wild-type, Tg2576⁺, hsp110^{-/-}, hsp110^{-/-}Tg2576⁺, hsp70I^{-/-}, hsp70I^{-/-}Tg2576⁺)×3 age groups=180 mice.

This example will also investigate whether the presence of Hsp110 and Hsp70i accelerates recovery after TBI, and if increasing the levels of Hsps accelerate recovery following TBI, as the expression of Hsps increases following environmental insults and because traumatic brain injury (TBI) is known to increase the risk for developing AD. TBI primarily results from falls, vehicle accidents, sport-related accidents and military injuries (Laird et al., 2008 Neurosignals 16:154-164). The prognosis for TBI patients remains poor because of the development of cerebral edema, elevated intracranial pressure, neuronal and vascular injury, and long-term cognitive dysfunction.

Studies using human and mouse models of TBI have shown that Ab production is increased following injury and there is evidence for increased amyloid deposition and risk of developing AD following TBI (Roberts et al., 1994 J Neurol Neurosurg Psychiatry 57:419-425; Gentleman et al., 2004 Forensic Sci Int 146:97-104; Tashlykov et al., 2007 Brain Res 1130:197-205; Uzan et al., 2006 Acta Neurochir (Wien) 148:1157-1164; Jellinger, 2004 Curr Opin Neurol 17:719-723; Raghupathi, 2004 Brain Pathol 14:215-222; Smith et al., 1999 J Neuropathol Exp Neurol 58:982-992; Nevin, 1967 J Neuropathol Exp Ne; Strich, 1970 J Clin Pathol Suppl (R Coll Pathol) 4:166-171; Tomlinson, 1970 J Clin Pathol Suppl 4:154-165). Therefore, understanding the underlying mechanism is critical for improving therapies. Hsp expression is enhanced following a variety of environmental insults such as physical and biochemical imbalance and ischemic injury including TBI (Huang et al., 2001 Mol Cell Biol 21:8575-8591; Morimoto, 1998 Genes and Development 12:3788-3796; Zhang et al., 2002 J Cell Biochem 86:376-393). Indeed, Hsp60 increase in cerebral spinal fluid of children following TBI and has been shown to a good biomarker correlating with the severity of the TBI (Lai et al., 2006 Dev Neurosci 28:336-341). Hsp70 has also been detected in the CSF of TBI patients. To understand the protective effects of Hsp110 and Hsp70i, we will use the Controlled Cortical Impact model of TBI (Laird et al., 2008 Neurosignals 16:154-164).

With this example, TBI injury will be administered to 8-12-weeks wild-type or hsp110^{-/-} (or hsp70i^{-/-})) male mice (Laird et al., 2008 Neurosignals 16:154-164; Wang et al., 2007 Exp Neurol 206:59-69; Lynch et al., 2005 Exp Neurol 192:109-116).

Experiment 8.1 will include an assessment of cerebral edema. Brain water content, an established measure of cerebral edema, will be quantified using the wet-dry method. At 24 hours (48, 72, 96, 120 hours will also be performed) post-injury, a time point associated with significant edema formation following experimental TBI (Griebenow et al., 2007 J Neurotrauma 24:1529-1535; Zweckberger et al., 2006 J Neurotrauma 23:1083-1093), brain water content will be estimated in a 3 mm coronal tissue section of the ipsilateral cortex (or corresponding contralateral cortex), centered on the impact site. Tissue will be immediately weighed (wet weight), then dehydrated at 65° C. The sample will be reweighed 48 hours later to obtain a dry weight. The percentage of water content in the tissue samples will be calculated using the following formula: {(wet weight−dry weight)/wet weight}×100.

Experiment 8.2 will determine the extent of TBI by quantitating the number of GFAP-positive cells in the brain sections at 0 days (no treatment) and at 1, 4, 7 and 28 days post-injury in wild-type, hsp110^{-/-} or hsp70i^{-/-} mice. Following experimental TBI or neurotrauma in human or experimental models, astrocytes undergo a change in phenotype named “reactive astocytosis”, a process which is characterized by cellular hypertrophy and hyperplasia, elongated cytoplasmic processes, and increased expression of GFAP (Laird et al., 2008 Neurosignals 16:154-164). Reactive astrocytes following TBI may have positive rather than a negative impact on TBI process (Morganti-Kossmann et al., 1999 J Neurotrauma 16:617-628). MMP3 is released by reactive astrocytes and is involved in the clearance of necrotic debris. The number of astrocytes expressing MMP3 will be quantitated in brain tissue sections by immunohistochemical staining. We will also perform immunoblotting of different brain regions (cortex, hippocampal area, medulla, cerebellum) using antibody to GFAP and MMP3 (Reeves et al., 2003. J Neurosci 23:10182-10189). Expression of other proteins such as TGFb (induces expression of GFAP, laminin and fibronectin) that limit axonal regeneration by generation of a glial scar will be determined by immunoblotting (127). The levels of Hsps (e.g., Hsp110, Hsp70i, Hsp60) will also be determined by quantitative immunoblotting. Since hsp110^{-/-} and hsp70i^{-/-} mice exhibit age-dependent p-tau, we will determine the extent of p-tau in wild-type and mutant mice 1-12 months following TBI using immunohistochemical staining of p-tau as presented in Example 1 and Experiment 6. The results will be compared between age matched untreated and treated groups.

Experiment 8.3 will determine the astrocytic glutamate transporter GLT1 ad GLAST expression, which regulate extracellular glutamate, by immunoblotting (Bullock et al., 1998 J Neurosurg 89:507-518). Glutamate excitotoxicity is critical in inducing neuronal damage following TBI. Concentrations of glutamate within the brain and cerebral spinal fluid (CSF) correlate with the severity of injury following TBI (Bullock et al., 1998 J Neurosurg 89:507-518).

With Experiment 8.4, the levels of TNFa in CSF at 0, 1, 7, and 28 days will be determined by ELISA (Hayakata et al., 2004 Shock 22:102-107; Kelliher et al., 1998 Immunity 8:297-303). Components of the inflammatory response are important but not entirely understood in TBI (Laird et al., 2008 Neurosignals 16:154-164) and Hsps play a role in inflammatory response (Calderwood et al., 2007. Ann NY Acad Sci 1113:28-39). TNFa which activates TNFR on glia and neurons is elevated in the CSF of neurotrauma patients within 24 hours post TBI.

Experiment 8.5 will determine whether absence of Hsp110 gene leads to increased APP processing and Aβ production, immunoblotting experiments as well as ELISA assays will be performed similar to those described in Aim 1, Experiment 6, at 0 days (before injury), and at 1, 4, 7 and 28 days following TBI, in wild-type, hsp110^{-/-} and hsp70i^{-/-} mice, as a number of studies have shown that β-amyloid and APP processing are increased following TBI.

In Experiment 8.6 motor and long-term neurocognitive deficit after TBI will be tested. Determination of behavioral parameters is an important tool for assessing the differences between groups of mice following TBI. To assess vestibulomotor function, we will perform Rotarod test before and on days 1 and 4 following TBI (Dineley et al., 2002 J Biol Chem 277:22768-22780) in mock-treated, and untreated wild-type, hsp110^{-/-} or hsp70i^{-/-} mice. To test long-term neurocognitive deficit, we will use a Morris water maze beginning at day 21 post TBI. Since the TBI model mice used here can be kept for many months, groups of mice mock treated, and TBI treated wild-type or hsp110^{-/-} mice at 8 weeks of age will be kept for 5-7 months and cognitive impairment will be determined. This time period has been selected since hsp110^{-/-} mice appear healthy at this time. However, this time period will be changed if we find hsp110^{-/-} mice to be more sensitive to TBI. If the results show significant impairment in hsp110^{-/-} mice compared to wild-type mice, we will then determine APP processing and Aβ production in these mice.

Experiment 8.7 will determine the extent of damage (infarct volume) following TBI. Mice will be analyzed using T2-weighted (high resolution) imaging to determine infarct volume before or following TBI at 0 days (before TBI) and at days 1, 4, 7 and 28 days post-TBI. The potential damaged site will also be imaged using diffusion tensor imaging and diffusion weighted imaging (DTI/DWI). Magnetic Resonance Imaging (MRI) analyses will be performed at the Small Animal Imaging at MCG. If the results of Experiment 8 using hsp110^{-/-} mice are relevant, appropriate Experiments described above will be performed using hsp70I^{-/-} mice.

Experiment 9 will determine whether administration of celasterol that increase expression of Hsps leads to reduced pathology following TBI. The levels of expression of Hsps such as Hsp110, Hsp70i, and Hsp25/27 (and others) are increased following exposure to environmental damage such as hyperthermia, oxidative stress, chemical and physical damage such as TBI (Huang et al., 2001 Mol Cell Biol 21:8575-8591; Morimoto, 1998. Genes and Development 12:3788-3796; Zhang et al., 2002 J Cell Biochem 86:376-393). We hypothesize therefore, that transient increase in the expression of Hsps in the cells (e.g., neurons) in the brain will protect them from apoptosis-induced following TBI. Celasterol, a member of triterpenoids family of compounds that are known for their anti-inflammatory properties (Westerheide et al., 2004 J Biol Chem 279:56053-56060), has been shown to enhance the expression of Hsps and in mouse models of Amyotrophic Lateral Sclerosis (ALS) they are able to block neuronal cell death and extend the life of these mice (Kiaei et al., 2005 Neurodegener Dis 2:246-254). Celasterol has been used in Chinese medicine and inhibits excessive cytokine production. Celasterol activates Hsps in comparable way as heat shock in contrast to other agents that increase the expression of Hsps with some delay (Westerheide et al., 2004 J Biol Chem 279:56053-56060). We will determine whether the transient activation of Hsps reduces the consequences of TBI.

Wild-type mice will be exposed to TBI or mock treatment (anesthesia but no TBI). Mice will either be sham treated or will be administered with daily doses of 8 mg/kg for 5 days (Kiaei et al., 2005 Neurodegener Dis 2:246-254). At 24, 48, 72, 96 and 120 hours, mice will be euthanized and wet/dry weight of impacted area will be determined (as in Experiment 8). At each time point brain tissue sections surrounding the injured area will be subjected to histology and immunohistochemical analyses to determine cell death (using TUNEL assay) and areas of necrosis. With a cohort of treated mice, immunoblotting experiments will be performed to detect induction of Hsps and expression of genes indicated in Experiments 8.1 to 8.5. If the initial damage appears to show significant changes between celasterol plus TBI treated versus TBI alone in wild-type mice, long-term evaluations of mice will be performed as outlined in Experiments 8.5-8.6. In addition, we will determine p-tau accumulation in the brain post TBI and compare the results in mice treated or untreated with celasterol.

Statistical considerations. All Experiments will be performed 3 times or with sufficient number of mice to generate results that are statistically unambiguous. Data will be expressed as mean+/−SEM. Differences between groups will be analyzed by Student's t test or ANOVA (for larger than two group comparisons). p values less or equal to 0.05 will be considered significant.

Mice needed. 20 mice for Experiment 8×4 time points×3 groups (+/+, hsp110^{-/-}, hsp70i^{-/-})=240 mice. For Experiment 9, we will need 40 mice×2 groups (treated and sham treated +/+males)=60 mice. Total 300.

Interpretation of Experiments 8 and 9. The above experiments will show the extent of injury, progression of disease, and final outcome of TBI in wild-type and hsp110^{-/-} (or hsp70i^{-/-}) mice. We have selected both biochemical as well as behavioral and MRI analyses to determine whether lack of Hsp110 leads to increased initial damage or reduced recovery in hsp110^{-/-} (or hsp70i^{-/-}) mice. If the data reveal significant findings and show Hsp110 is protective following TBI, comparable studies will be performed using hsp70^{-/-} mice, since this Hsp member is also highly inducible in the brain following stress, and therefore, its levels can be manipulated by administration of drugs such as celasterol following TBI (Zhang et al., 2002 J Cell Biochem 86:376-393; Kiaei et al., 2005 Neurodegener Dis 2:246-254). For Celaterol treatment, we have selected 8 mg/kg per mice daily dose as it has been reported previously to cause no toxicity. However, the duration of the treatment can be extended for several months given in daily food if we determine beneficial effects of Celasterol treatment on TBI in mice. Initial experiments using celasterol treatment of TBI treated mice will be with wild-type only. However, if appropriate, hsp110^{-/-} and hsp70i^{-/-} mice to determine if expression of one of these Hsp is affected, how effective celasterol treatment would be in reducing TBI's harmful effects. Determination of p-tau in wild-type, hsp110^{-/-} or hsp70i^{-/-} mice following Celasterol treatment following TBI will reveal whether enhanced expression of Hsps reduce expression of p-tau following TBI. If Celasterol treatment post TBI reduce its harmful effects future directions could include findings drugs that may be more effective in enhancing the expression of Hsp110 and/or Hsp70 following TBI and testing these drugs in the preclinical or clinical setting.

Methods. MRI analyses and behavioral studies will be performed in the MCG Core facilities. Closed head injury model of TBI: mice will be anesthetized using Avertin. Rectal temperature will be maintained at 37° C. The animal will be placed in a stereotactic device, the scalp will be incised and skull exposed. TBI will be applied using a 2 mm diameter pneumatic piston (Air-Power, Inc. High Point, N.C.) (Griebenow et al., 2007 J Neurotrauma 24:1529-1535; Zweckberger et al., 2006 J Neurotrauma 23:1083-1093). The impactor is discharged at 6.8+/−0.2 m/s with the head displacement of 3 mm. After completion of the procedure animal will be allowed to recover. With this procedure mortality is less than 2% and mice can live almost normal life span. Sham treated animals will not receive the TBI but will receive other procedures before TBI. Immunohistochemical staining and immunoblotting of brain sections will be as previously described (Homma et al., 2007 J Neurosci 27:7974-7986). APP processing and Aβ production will be performed as described in Experiment 6 and 7. For MRI; two techniques that are commonly applied to TBI imaging are T2-weighted imaging (T2W) and diffusion-weighted imaging (DWI). Both techniques are capable of offering complimentary pathologic information. T2W images are sensitive to edema in general, as tissues with a high fluid content (e.g., cerebrospinal fluid) tend to have an increased T2 relaxation rate and correspondingly higher signal in T2-weighted images. Use of DWI to calculate a map of the apparent diffusion coefficient (ADC) of water in the brain, which is one of the current clinical standards, can provide insight into local cell integrity (Falangola et al., 2007 NMR Biomed 20:343-351). Due to the differences between DWI/ADC and T2W images, DWI imaging is useful for separating cytotoxic edema (dark on ADC maps) from vasogenic edema (bright on ADC maps). Both forms of edema are generally bright on T2W images, in proportion to the relative volume of liquid. Use of diffusion tensor imaging (DTI) to calculate a more representative measurement of mean ADC also provides additional monitoring of white matter integrity using a fractional anisotropy map (FA). Celasterol will be purchased from MicroSource, Inc. Conn., Celasterol will be dissolved in DMSO at 50 mg/kg and diluted in saline before intraperitoneal administration (Kiaei et al., 2005 Neurodegener Dis 2:246-254). Sham treated mice will receive appropriate dilutions of DMSO.

This example will examine brain tissue sections from healthy or AD patients to determine the locations of Hsp110, Hsp70i, tau, p-tau, Pin1, and Ab in neurons, and in senile plaques and/or NFTs and determine whether the components of NFTs contain Hsp110.

Experiment 10. Previous studies have shown that Hsp70i, Hsc70, Hsp90, Chip, and ubiquitin as well as p-tau are found in NFTs in AD brain (Petrucelli et al., 2004 Hum Mol Genet. 13:703-714). Since Hsp110 may also be present intracellularly or in plaques that are present in AD brain, we will survey commercially available slides containing different segments of normal and diseased human brain tissues (BioChain, CA or Cybrdi, Inc.) (e.g., hippocampal area) to detect expression of Hsp110, Hsp70i, Hsc70, p-tau, total tau, and Pin1 (as well as Hsp70, Hsp90, CHIP and ubiquitin) using the appropriate antibodies. These experiments are critical since the results will show whether Hsp110 is also associated with other molecular chaperones in senile plaques and NFTs in AD brain, or if the role of Hsp110 is similar or different from the presumed function of other molecular chaperones (e.g., protein disaggregation, facilitating protein degradation (Dickey et al., 2006 J Neurosci 26:6985-6996)). Since immunohistochemical staining of AD brain will not show whether there are physical interactions between various Hsps (Hsp110, Hsp70i, Hsc70, Hsp90) with NFTs, we will prepare soluble, Sarkosyl soluble, and pellet fractions of AD and healthy brain extracts and immunoblotting of each fraction will be performed to detect the Hsps, as well as other components such as CHIP, Pin1, tau, p-tau, APP and Aβ. In addition, we will perform immunoprecipitation analyses using antibody to tau or p-tau and determine whether Hsp110 as well as other Hsps (Hsp70, Hsc70, Hsp90), Pin1 and CHIP are associated with tau or p-tau in these samples.

Statistical analyses. all experiments will be performed at least three times using material from at least three healthy and AD donors.

Interpretation of Experiment 10. The studies of Experiment 10 will determine the expression pattern of Hsp110 in healthy and AD brain sections. Additional studies such as immunoprecipitation of Hsp110, other Hsps, or p-tau will also be performed with brain extracts of patients and healthy brains to determine whether Hsp110 or other Hsps coimmunoprecipitate and the relationships between Hsp110 or other Hsps to p-tau (and Aβ) in the diseased brains.

Methods for Experiment 10. Immunohistochemical staining of human brain tissue sections will be performed as previously described (Homma et al., 2007 J Neurosci 27:7974-7986; Lopes et al., 2008 Glia 56:1048-1060). Both the concentrations of the primary antibodies and secondary antibodies will be extensively tested in order to ensure absence of nonspecific binding. We will perform immunoprecipitation experiments using antibody to total tau or p-tau using AD or healthy brain extracts followed by immunoblotting. Future directions could include proteomics analyses of human brain cell lysates following immunoprecipitation of Hsp110, tau, or p-tau in order to identify novel components of molecular chaperone machines that are part of the tau metabolic pathways.

Statistical considerations. All experiments will be performed three times. Samples from at least 10 AD and 5 healthy brains will be used. Immunoblots will be quantitated by densitometry (Homma et al., 2007 J Neurosci 27:7974-7986). Data will be expressed as mean+/−SEM. Differences between groups will be analyzed by Student's t test (or ANOVA as appropriate). p values less or equal to 0.05 will be considered significant. In all cases group sizes will be selected to produce results that are statistically unambiguous.

This example will also use cerebral spinal fluid from the patients who have received traumatic brain injury to examine the levels of Hsp110 or Hsp70i to determine whether the level of these Hsps are elevated in CSF following injury. The Medical College of Georgia is accredited as one of only three Level I Trauma Centers in the State of Georgia. This facility routinely treats 300-350 neurotrauma patients annually. Patient specimens will be collected via an external ventricular drain (EVD) from any patient requiring CSF diversion, including neurotrauma, spontaneous intracerebral hemorrhage (ICH), intraventricular hemorrhage (IVH), intraparachymal hemorrhage (IPH), subarachnoid hemorrhage (SAH), brain tumor, and normal pressure hydrocephalus (NPH) patients. NPH patients will serve as an ideal control population as these patients exhibit significant edema without the initial traumatic event (therefore, the excitotoxicity component will be removed from these patients). Other neurological injuries, including ICH, IVH, IPH, SAH and brain tumor patients, will be used to assess the specificity for Hsp110 and Hsp70i release to neurotrauma. Additionally, blood will be collected from neurotrauma (and other brain injuries) patients upon admission in the emergency room and routinely thereafter to monitor for various physiological parameters (e.g. arterial blood gases, electrolyte balance, coagulation parameters, white blood cell count, hemoglobin). All de-identified patient specimens will be coded by the attending physician, so experiments will be conducted in a double blinded manner and the laboratory staff will never have access to patient information other than diagnosis. Upon the conclusion of data collection, the samples will be decoded for statistical analysis. The collection of CSF and blood for the screening of novel biomarkers of head injury was previously approved by the institutional IRB committee and will not influence or alter patient care in any way.

Hsp110 and Hsp70i levels in patient CSF and serum will be determined by quantitative immunoblotting, using a series of well characterized mono or polyclonal antibodies to Hsp110 and Hsp70i (Stressmarque). Hsps quantification will be performed by comparing patient values following densitometry analysis within the linear range of a standard curve generated by loading known amounts of recombinant individual Hsps. A commercially available enzyme-linked immunoassay (EIA) for Hsp70i, which specifically detects each Hsp in biological samples (e.g CSF, serum), will be used as an alternate method to confirm the Immunoblotting data. We will initially test 10 samples as pilot studies. We will then analyze a sample size analyses to determine whether it is a sufficient size. However, this sample size is in line with previous studies of novel biomarkers in the CSF of trauma patients (Morganti-Kossmann et al., 1999 J Neurotrauma 16:617-628; Hecker et al., 2008 Cell Stress Chaperones 13:435-446) and could easily be achieved given the large number of patients within our institution. Upon the collection of the data from a larger cohort, we plan to perform a post-hoc power analysis to ensure adequate statistical power to improve confidence in the clinical extrapolation of these findings. To determine whether serum/CSF levels of Hsp110 and Hsp70i levels retrospectively correlate with the clinical condition (e.g Glasgow Coma Scale (GCS) score (TBI severity), Glasgow Outcome Score (GOS; measure of neurological outcome), ICP (measure of cerebral edema severity)), a Pearson's correlation test will be performed, as described previously for clinical studies of CSF after neurotrauma (Harris et al., 2009 J Neurosurg 110(6):1322; Tasaki et al., 2009 J Trauma 66:304-308).

Interpretation of Experiment 11. Expression of Hsp70 has been shown in the CSF following TBI (Kumar et al., 2007 Hum Mol Genet. 16:848-864). This protein as well as expression of Hsp60 that previously been shown following TBI in children will be used as positive controls. Expression of Hsp110 in the CSF has not been previously analyzed in the patients. If necessary expression of other proteins such as S100B will be used as a positive marker (Hayakata et al., 2004 Shock 22:102-107). If good correlations are observed between the level of Hsp70 or Hsp110 in the TBI patients and the extent of injury, release of these proteins in the CSF can be used as pathological marker for TBI. Results of these experiments will be compared and correlated with the results of our animal studies and experiments can be further refined using our mouse models. However, the Hsp110 ELISA is not presently commercially available. If there are excellent correlations found between one or both Hsps in the CSF of patients exposed to TBI, future directions could use the level of these Hsps as biomarkers for determining the severity of TBI.

Methods for Experiment 11. For Immunoblotting, 30 mg of protein from each CSF sample will be used to determine the level of Hsp110 and Hsp70 (Hayakata et al., 2004 Shock 22:102-107). Quantitation will be performed against known amounts of purified Hsp110 or Hsp70 (Harris et al., 2009 J Neurosurg 110(6):1322; Tepas et al., 2009 J Pediatr Surg 44:368-372; Tasaki et al., 2009 J Trauma 66:304-308). Purified Hsp70 will be purchased from Stressmarque. Antibodies to Hsp110, Hsp70 and other Hsps will be purchased from assaydesign, BD Biosciences, or MBL International. Hsp70 ELISA will be purchased from assaydesigns. Hsp110 will be purified by standard methods in our laboratory and since there are many excellent antibodies to Hsp110, an ELISA will be set to detect this protein in CSF (Schmidt et al., 2005 Methods Mol Biol 299:279-297).

### Example 6

### Functional Contribution of Hsp70i, Hsc70 or Hsp25 in Tumor Vasculogenesis and Angiogenesis

Angiogenesis from blood vessels adjacent to growing tumor cells is required for tumor malignancy and metastasis. Therefore, inhibition of angiogenesis using drugs that act on endothelial cells is an attractive therapeutic strategy and several inhibitors are under clinical trial (Neri and Bicknell, 2005 Nat Rev Cancer 5:436-446; Tozer et al., 2005 Nat Rev Cancer. 5:423-435, Kerbel and Folkman, 2002 Nat Rev Cancer 2:727-739). Clearly, there is urgent need for more effective angiogenesis inhibitors targeting not only endothelial cell differentiation, leading to formation of primitive endothelial tubes, but other cells such as pericytes whose differentiation to vascular smooth muscle cells is required for maturation of primitive tubes to fully developed blood vessels. Our findings and evidence in the literature suggest that Hsps likely play a critical role in vascular system development, and thus targeting specific Hsps may be relevant for tumor therapy. In particular, Hsp70i and Hsc70 are expressed in vascular endothelial cells and, in the context of blood vessel formation, their anti-apoptotic property is likely important in promoting endothelial cell survival (Portig et al., 1996 Electrophoresis 17:803-808; Kabakov et al., 2003 Cell Stress Chaperones 8:335-347). A role for Hsp25 in blood vessel formation is supported by data demonstrating that VEGF increases migration and induces reorganization of the microfilament network in human endothelial cells in a manner dependent on p38 MAP kinase activation and phosphorylation of Hsp25 (Landry and Huot, 1999 Biochem Soc Symp 64:79-89, Rousseau et al., 1997 Oncogene 15:2169-2177; Huot et al., 1997 Circ Res 80:383-392; Keezer et al., 2003 Cancer Res 63:6405-6412). Based on our data with Hsp25-LacZ reporter mice showing that it is also expressed in smooth muscle cells of the developing cardiovascular system, we propose that Hsp25 may function both in regulating endothelial cell migration and pericyte development to vascular smooth muscle, and these properties will be evaluated.

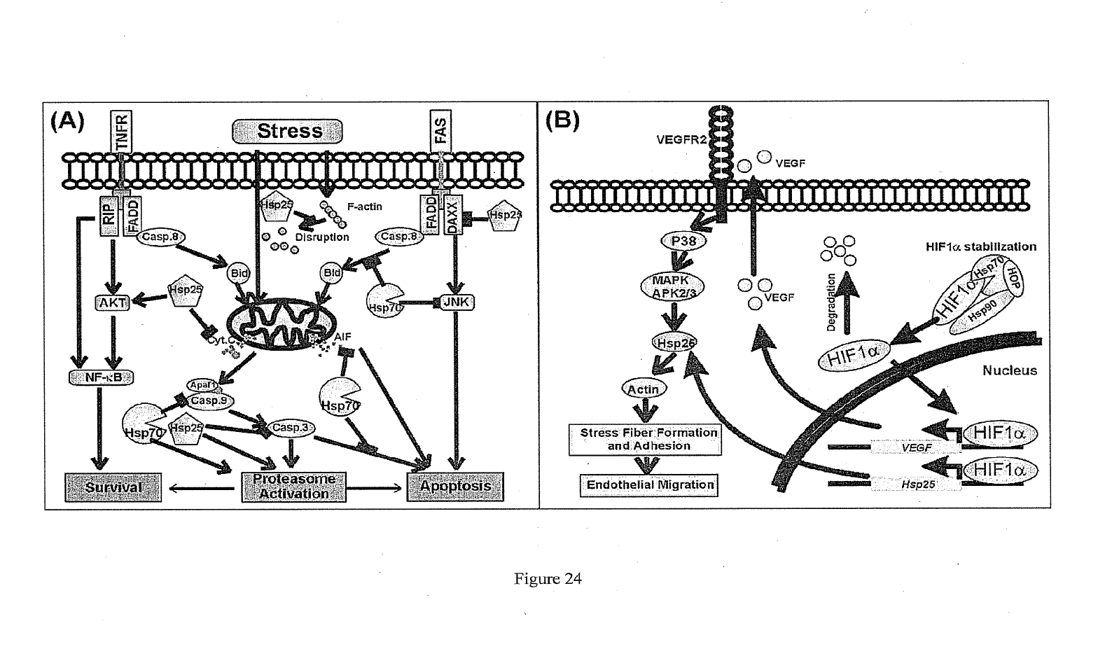

Experiments described in this example are directed to test if Hsp70i, Hsc70 and Hsp25 chaperones are crucial for microvascular endothelial cell growth and hence tumor angiogenesis, and if their disruption in vivo may inhibit tumor growth. The specific premise to be tested is that deletion of these Hsps individually or in combination will disrupt proper development of vasculature, based on their function as potent negative regulators of apoptosis induced by stress stimuli in endothelial cells and positive regulators of HIF-1 activity and endothelial cell migration (for detail see FIG. 24).

We plan comparative studies on the roles of Hsp70i, Hsc70 and Hsp25 in formation and maintenance of tumor vasculature and tumor growth in well-established animal models. Subsequent studies on endothelial cells generated from aorta of wild-type or Hsp-deficient mice will investigate the mechanisms by which Hsps may modulate blood vessel formation. Specifically, we postulate that an intrinsic difference in endothelial function in Hsp-expressing endothelial cells may account for impaired in angiogenesis in Hsp70i, Hsc70 or Hsp25 deficiency and this aspect will be evaluated as detailed in the below.

Physiological significance of Hsp70i, Hsc70 or Hsp25 in initiating and sustaining a functional tumor vasculature and tumor growth in animal models. These studies will define the role of Hsp70i, Hsc70 or Hsp25 expression by endothelial cells in formation of tumor vasculature and thus tumor growth in vivo. Conditional Cre-loxP based gene knockout technology, combined with hematopoetic repopulation experiments and tumor transplantation experiments, will be used to separate the relative contribution of major Hsp effector mechanisms during differentiation of hematopoetic bone marrow derived endothelial progenitor cells to mature endothelial cells incorporated into the developing neo-vasculature of growing tumors, independently from their pro-survival function on tumor cells. In particular, the feasibility of manipulating tumor vascularization by altering Hsp expression either in bone marrow precursor or differentiating endothelial cell populations will be probed.

Experiment 1 will determine whether Hsp70i deficiency affects tumor growth. Hsp70i−/−, Hsp70i+/−, and B6 control mice will be implanted with syngeneic tumors (K1735-melanoma, MB49 bladder cancer, B16-F10-melanoma, and LLC-lewis lung carcinoma) by subcutaneous injection into hind limb flanks and tumor growth will be measured for 30 days. Tumor vascularization will be quantitated by counting CD34+ vessels in 5 sections of each tumor per group of 5 mice in selected fields of high vascular density in sections immunostained for CD34 and scanned by confocal microscopy. This analysis will be complemented by double-staining tumor sections with antibody to CD34 and TUNEL to detect apoptosis in the host endothelium. Before proceeding with this analysis, tumor implantation conditions will be optimized by dose-response and time course analysis. To further confirm that impaired microvessel proliferation may limit tumor growth in Hsp70i−/−mice, angiogenesis in a tumor independent model involving subcutaneous implementation of reconstituted basement membrane (Matrigel) containing angiogenic factors (basic fibroblast growth factor (bFGF) or vascular endothelial growth factor (VEGF) will be studied (Egami et al., 2003 J Clin Invest 112:67-75; Saadoun et al., 2005 Nature 434:786-792; Chen et al., 2005 Nat Med 11:1188-1196). For in vivo Matrigel assay, Hsp70i−/− or B6-control mice will be anaesthesized and receive two 0.5 ml injections of Matrigel (with or without bFGF or VEGF) under abdominal skin. Matrigel pellets will be harvested after 5 days, digested with dispase and hemoglobin content, (a measurement for intact vessel formation) will be determined by Drabkin's method (Ricca Chemical Company). In addition, histological examination after staining with Hematoxylin and eosin (H&E) will be performed to quantitate vessel-like structures in the growth-factor-supplemented Matrigel from Hsp70i−/− and wild-type mice.

The next set of analyses will evaluate the contribution of bone marrow (BM) derived stem cells from Hsp70i deficient mice in tumor angiogenesis. Studies have demonstrated that BM contributes different cell types to tumor stroma, including hematopoetic cells, and vascular endothelial cells. The existence of endothelial precursor cells and their involvement in vessel formation is widely accepted (Rafii and Lyden. 2003 Nat Med 9:702-712, Rafii et al., 2002 Nat Rev Cancer 2:826-835; De Palma et al., 2003 Nat Med 9:789-795). However, as this cell population is not well characterized and it is unclear whether endothelial progenitor cells (VEGFR2-positive) or hematopoetic (i.e. CD31−, CD45+, CD11b+, TIE-2+) mononuclear cells contribute to tumor neovasculogenesis, we will use lineage negative cells (CD45−, CD11b−, B220− cells) for our BM reconstitution experiments. If differences in tumor growth are observed between mutant and control mice, we will evaluate whether they correlate with endothelial cell differentiation defects and reduced survival, and examine whether the capacity of Hsp70i−/− BM to promote tumor vascularization is compromised. To this end, B6-mice lethally irradiated (8.5 Gy) and reconstituted with 107 BM cells from Hsp70i−/− or Hsp70i+/− or B6 mice will be implanted with tumor cells and the kinetics of tumor growth and degree of vascularization will be evaluated as detailed above.

For further studies, transplantation of endothelial and non-endothelial BM cell lineages will also be considered; this however depends upon the outcome of the above analyses. In this case, lineage-negative cells (1×106 cells/mouse) will be injected into the tail vein of lethally irradiated B6 mice to allow full engraftment of transplanted hematopoietic stem cells, and the effects of BM transplantation on growth of tumors (listed above) implanted into engrafted mice 8-12 weeks later will be measured. For this analysis, lineage negative cells in bone marrow will be purified with a kit (Stem Cell Technologies) following manufacturer's instructions. Note that lineage negative cells flow through and can be separated into Sca-1+ (endothelial precursor cells) or Sca-1− hematopoetic stem cells (HSC) using a MoFlo cell sorter (MCG-cytometry core facility). This protocol has been used successfully to define BM derived cell populations in tumor vasculogenesis (De Palma et al., 2003 Nat Med 9:789-795). In addition, we will use donor or host cells congenic for the CD45 locus, so their repopulation frequency can be assessed using antibodies that recognize donor (CD45.1) and host (CD45.2) markers respectively. Finally, for therapeutic exploration it is worthwhile examining whether tumor vascular endothelium derived from Hsp70i deficient BM cells exhibits increased sensitivity to stress stimuli (e.g. heat, irradiation). This possibility is strongly supported in the literature, and suggests; that hyperthermic treatment may improve the clinical outcome of cancer, especially if this treatment is combined with ionizing irradiation (IR) or chemotherapy (Hildebrandt et al., 2002 Crit. Rev Oncol Hematol 43: 33-56); that endothelial apoptosis can determine the tumor response to IR treatment (Garcia-Banos et al., 2003 Science 300:1155-1159); and that Hsp70i ablation results in increased sensitivity of mouse embryonic fibroblast cells to IR exposure (Hunt et al., 2004 Mol Cell Biol. 24:899-911). Full time courses (6, 24 and 48 hrs for heat and 1-10 h for IR treatment) and dose responses to heat (39-42° C. for 1 h) or IR treatment (single dose x-ray radiation of 1-20 Gy) will be required to accurately determine whether baseline endothelial cell apoptosis increases in tumor implanted Hsp70i−/− mice relative to Hsp70i+/+ controls. It is anticipated that Hsp70 deficiency will render tumors sensitive to endothelial apoptosis and promote a positive outcome. Successful completion of this study will include similar analyses carried out with lethally-irradiated animals transplanted with BM of the opposite genotype (Hsp70i−/− marrow into Hsp70i+/+ mice and vice versa). However, we will prioritize experimental design. Thus, if analyses with non-irradiated mice indicate that Hsp70i deficiency significantly impacts tumor growth, subsequent analyses will dissect the specific contribution of BM derived cells to this process.

Experiment 2 will assess the contribution of Hsc70 in tumor growth and neo-vasculogenesis is as described in detail above (Exp. 1) with the following modifications. Due to embryonic lethality associated with complete loss of Hsc70, it is impossible to use Hsc70−/− mice as recipients for tumor implantation studies or as a source for BM cells in reconstitution and tumor engraftment experiments. Use of mice with a conditional Hsc701oxP allele circumvents this problem, enabling tumor studies with mice reconstituted with BM cells where Hsc70 is effectively deleted by transient Cre expression. We will use two experimental systems to evaluate the role of Hsc70 in tumor growth associated with neo-vasculogenesis. The first set of experiments will be performed with lethally irradiated B6-mice reconstituted with BM (107 whole BM or 106 lineage negative) in which Hsc70 was deleted by infection with Cre-expressing adenoviruses. The animals will be implanted with different tumors and the kinetics of tumor growth and degree of vascularization will be evaluated as detailed above. Full engraftment of transplanted BM cells will be assessed by in vitro clonogenic assay (Huang et al., 2001 Mol Cell Biol 21:8575-8591), and the contribution of BM to sustain microvascular function and thus growth of implanted tumors will be examined. The in vitro clonogenic assay is well established.

A second set of experiments will be performed by inducible Hsc70 ablation in BM reconstituted animals before and after tumor implantation. Hsc70^{loxP} mice will be bred with Rosa26-^{CreTR} T2/LacZ transgenic mice and used as BM cell donors to repopulate lethally irradiated host mice. Hsc70 will be deleted by treatment with tamoxifen (oral or i.p.) (Seibler et al., 2003 Nucleic Acids Res 31:E12). Although this approach has been successfully used for conditional gene deletion studies, we need to optimize the treatment for our experiments. This will be done by measuring Cre recombination events allowing ablation of Hsc70 gene, which for the Rosa26-CreER T2/LacZ mice can be easily monitored by lacZ reporter expression, in time course and dose response analyses. We plan to carry out tumor studies in mice where Hsc70 deletion was completed before tumor transplantation (treatment from −5 to 0 days), during onset of tumor growth (+7 to +12 days) or at a late stage (+15 to +20 day).

Experiment 3 will examine the contribution of Hsp25 in tumor growth by regulating neovasculogenesis are proposed. The design and execution of these studies follow that described above for Hsp70i. Further analyses to determine functional synergism among Hsp70i, Hsc70 and Hsp25 in tumorigenesis using Hsp70i−/− BM donor cells in combination with endothelial cell-specific deletion of Hsc70 or Hsp25 may be considered upon the outcome of the above experiments.

Mechanisms for impaired tumor angiogenesis in Hsp70i, Hsc70 or Hsp25 deficiency. We anticipate that the studies proposed above will confirm the significant contribution of major molecular chaperones to tumor microenvironment development and especially tumor blood vessel formation. To explore possible mechanisms, we propose that intrinsic differences in endothelial cells may account for the impaired angiogenesis in Hsp-deficiency in vivo. This is based on Hsp function as potent negative regulators of apoptosis induced by stress stimuli in endothelial cells and/or positive regulators of HIF-1 activity and endothelial cell migration (for detail see model FIG. 24).

To test this hypothesis, intrinsic endothelial cell functions required for angiogenesis will be studied using cultured endothelial cells generated from mouse aorta (Saadoun et al., 2005 Nature 434:786-792, Yao et al., 1999 Blood 93:1612-1621). This analysis will provide important information regarding the molecular and morphological events of endothelial cell development and function where these chaperones are involved.

Experimental design. Our basic strategy is to study Hsp-function on two levels: We will first explore the effects of Hsp ablation on endothelial cell function under physiological conditions, and then analyze pathways modulated by Hsps under stress-induced conditions, as outlined in FIG. 24. FIG. 24 shows a simplified schematic model for the function of Hsp70i/Hsc70 and Hsp25 in vasculogenesis and angiogenesis under investigation in this specific aim. FIG. 24A shows a possible scenario is that Hsp70i, Hsc70 and Hsp25 act in a cytoprotective manner promoting blood vessel formation via inhibition of general apoptosis pathways and interfering with key apoptotic protein expression induced by heat shock or other stress situations in endothelial cells (Concannon et al., 2003 Apoptosis 8:61-70; Gamido et al., 2003 Cell Cycle 2:579-584; Beere, 2004 J Cell Sci 117:2641-2651). FIG. 24B shows another possible scenario is that these Hsps act either in stabilizing HIF-1 activity (Hsp70i, Hsc70) and/or regulating endothelial cell migration (Hsp25). For HIF-1α stability the emerging model is that interaction of HIF-1α with the Hsp90/Hsp70 heterocomplex prevents its degradation under hypoxic conditions by nonpVHL-mediated ubiquitination and proteasomal degration (Zhou et al., 2004 J Biol Chem 279:13506-13513; Katschinski et al., 2004 Cell Physiol Biochem 14:351-360). Activation of P13-kinase/Akt signaling results in HIF-1α induction and Hsp90/Hsp70 expression to protect HIF-1α from degradation. In addition, activation of the SAPK2/p38 pathway by mitogenic signals such as VEGF, downstream from HIF-1 activity, leads to phosphorylation of Hsp25 and increased actin polymerization. This regulates vascular endothelial cell migration and is a proposed regulatory pathway through which angiogenic inhibitors control the angiogenic switch (Rousseau et al., 1997 Oncogene 15:2169-2177; Rousseau et al., 2000 Trends Cardiovasc Med 8:321-327; Rousseau et al., 2000 J Biol Chem 275:10661-10672; Cross et al., 2003 Trends Biochem Sci 28:488-494).

There is also evidence that Hsp27 expression is regulated by HIF-1 (Whitlock et al., 2005 Invest Opthalmol V is Sci 46:1092-1098). Multiple and complex interactions between Hsps and cellular regulatory networks are likely involved to functionally promote endothelial blood vessel formation. Therefore experiments will explore Hsp function along major pathways known to play a role in regulation of apoptosis and angiogenesis. Finally, fine analyses, including activation profiles of additional molecules involved in apoptosis or angiogenesis, may be required to delineate target molecules for potential functional deficits observed in Hsp-deficient endothelial cells. Initial studies will address the contribution of Hsp70i to angiogenesis, and subsequent studies will evaluate the contribution of Hsc70 or Hsp25 by specific deletion in vascular endothelial cells.

Experiment 1a will examine vascular endothelial cell functions in Hsp70i−/−, Hsp70i+/− or wild type B6-derived endothelial cell cultures.

We will define the contribution of Hsp70i in endothelial cell function under physiological conditions: Cultured endothelial cells will be examined for morphological appearance and growth. Next, endothelial cell functions required for angiogenesis, such as proliferation, adhesion and migration will be examined. Comparative measurements of cell growth, adhesion, and migration will be assayed following published procedures (Miao et al., 2001 Cancer Res 61:7830-7839). Cell adhesion will be quantified from the number of endothelial cells adhering to a gelatin support within 4 h of plating, and cell migration, towards varying concentrations of fetal bovine serum, a potent chemotactic stimulus, will be quantified with a Boyden chamber. For proliferation, the number of cells will be determined with a chromogenic assay kit in 96-well plates (Promega). Finally, cord formation, a multistep process that includes migration, and formation of cord/tube like endothelial cell structures will be studied in Matrigel-coated wells (Troyanovsky et al., 2001 J Cell Biol 152:1247-1254). Cord formation will be quantified at 24 h by calculating the average tube length multiplied by the total number of tubes in a particular optical field. In addition, the number of small-diameter or large-diameter structures will be determined.

The second set of analyses will determine the susceptibility of differentiated endothelial cells from Hsp70i−/−, Hsp70i+/− or wild type B6 mice to apoptosis induced following exposure to different stress-inducers (heat shock, TNF-α, Fas-ligation, or hypoxia). As a readout of apoptosis we will assay several parameters, including PARP cleavage (by immunoblotting), caspase 3 activation (by fluorometric assay), TUNEL assay, Annexin V staining, cytochrome C release and activation of stress-kinase (by immunoblotting). All these methods are routinely used in the PI's and Co-PI's laboratories. For heat sensitivity and thermotolerance studies, cells will be exposed to heat shock and 6 or 24 hours later endothelial cell apoptosis will be quantitated by TUNEL or Annexin V staining and analysis by FACS. Thermotolerance will be studied on cells preconditioned with a relatively mild heat shock (43° C. for 20 min) and allowed to recover at 37° C. for 6 or 24 h before challenge with a lethal heat shock (45° C. for 30 min). Cell viability and the level of apoptosis after heat challenge will be measured following further recovery at 37° C. for 24 h. To study effects of TNF-α treatment or Fas-ligation, cells will be treated with a range of concentrations of TNF or Fas antibody, and apoptosis will be measured 24 and 48 hours later. To analyze the response to hypoxia, cells will be exposed to low oxygen (2%, 0.5% or 0.01%) for various time periods and apoptosis will be measured. For this assay differentiated endothelial cells will be placed in an adjustable hypoxia chamber with real time pO₂ readout (Invivo 200; Biotrace International). Based on Hsp70i expression pattern we predict that under this treatment regime depletion of Hsp70i will affect endothelial cell survival. These analyses will produce information regarding the protective role of Hsp70i in pathologic stress situations during vascular endothelial cell development.

Finally, the effects of Hsp70i ablation on cell function will be further studied by measuring HIF-1α activity in endothelial cell cultures for various time periods under noimoxic (O₂ concentration 21%) or hypoxic (O₂ concentration of 2%, 0.5% or 0.01%) conditions. HIF-1α protein levels will be analyzed by immunoblotting. HIF-1α can be detected under normoxic condition using relatively large amounts of protein extract whereas under hypoxic conditions its detection requires much less protein. In addition, expression of VEGF, a major angiogenic factor regulated by HIP-1α, will be determined at mRNA and protein levels. This study will provide us with information to assess the role of Hsp70i, perhaps through its incorporation in Hsp90/Hsp70 heterocomplexes, in preventing degradation of HIF-1α under hypoxic (VHL-independent) conditions.

Experiment 2a will determine the contribution of Hsc70 to endothelial cell function. The experimental design and analyses parallel those described for Hsp70i above. As a control, Hsc70i+/− or wild type B6 endothelial cell cultures will be studied in parallel. Due to constitutive high level Hsc70 expression in endothelial cells, it is possible that its deletion may adversely affect cell physiology in culture. To this end, we will derive endothelial cells from Hsc70^{flox/flox} mice and delete Hsc70 in vitro by transfection with plasmids (CMV-Cre) or infection with adenovirus expressing Cre-recombinase. As a control we will infect parallel cultures with a LacZ expressing adenovirus. Efficient recombination and deletion of Hsc70 will be confirmed by PCR. Due to high adenovirus infection efficiency and high level of Cre expression, deletion efficiency should be very high (>90%)

Experiment 3a will determine the effects of Hsp25 deletion on vasculature development in vitro. Isolation, functional characterization and culture of Hsp25−/− endothelial cells and the experimental design and analyses proposed to address this issue are essentially as described for Hsp70i above.

These experiments will provide compelling evidence for the involvement of Hsp70i, Hsc70 and Hsp25 in endothelial cell physiology, offering a mechanistic explanation for impaired angiogenesis in Hsp-deficiency and thus inhibition of tumor growth in vivo. Also working with cultured cells rather than tissues should allow more detailed quantitative analyses. Ablation of Hsp70i, Hsc70 or Hsp25 genes, especially in combination, may adversely affect physiology and perhaps survival of endothelial cells in culture, and it would be of great interest to know at which level cell function is inhibited. To circumvent complications associated with constitutive gene deletion and for further functional analysis, we may consider using an inducible Cre-loxP system. In this case, endothelial cells generated from mice carrying Hsc70^{flox/flox} or Hsp25^{flox/flox} alleles and transgenic for Cre recombinase, the expression of which is either controlled by an interferon (IFN)-α inducible promoter (Mx1-Cre) or is tamoxifen induced (Rosa26-CreERT2/lacZ reporter; estrogen receptor) will be transiently exposed to IFN-α or tamoxifen resulting in Hsc70 or Hsp25 gene ablation at high frequency (Seibler et al., 2003 Nucleic Acids Res 31:E12, Kuhn et al., 1995 Science 269:1427-1429; Guo et al., 2002 Genesis 32:8-18).

A key factor that may complicate the above analyses is the fact that Hsp-mediated regulation of endothelial function may involve several parallel signaling pathways and the function of each Hsp chaperone under study may be compensatory or even overlapping. Although this is a concern and challenge for interpreting data from Hsp-deficient cell culture studies, we are confident that the proposed analyses will produce interpretable data to conclude how Hsps contribute to endothelial cell differentiation and blood vessel formation. Clearly, comprehension of the mechanistic basis for Hsp function in endothelial cell death and cell survival will require further detailed biochemical and molecular definition of target molecules and pathways regulated by these molecular chaperones. One approach to this complex issue will be to compare the induction of various members of the apoptotic cascade, (such as receptor dimerization, procaspase/caspase recruitment to the receptor complex, dATP/Cytochrome C/Apaf-1/caspase 9 complex formation etc.) in cells where Hsps are intact or deleted in various combinations. This investigation will provide further important insights into the physiological function of these key genes.

This example will not only define the contribution of major molecular chaperones to tumor microenvironment development and especially tumor blood vessel formation, but should also produce important data regarding their role in tumor response to stress stimuli. We anticipate a decreased ability of Hsp-deficient BM cells to promote angiogenesis and thus rapid tumor growth based on two possible scenarios; (1) that Hsp-deficient BM cells transferred into lethally irradiated recipients fail to reconstitute the hematopoietic compartment resulting in death of the mice. Based on the fact that Hsp70i−/− or Hsp25−/− mice are viable this is unlikely. Potential mechanisms that deserve consideration for further analysis are that the deficit in repopulation potential of Hsp-deficient BM cells may result from impaired homing into host BM or increased cell death (apoptosis); (2) Hsp-deficient BM cells exhibit a normal capacity to repopulate host hematopoietic system, but fail to sustain the tumor microvascular environment, perhaps by an increased baseline apoptosis or failure to home to tumor. The extent to which different tumors utilize BM endothelial precursors as opposed to the recruitment of mature endothelial cells from the circulation is unknown. Thus it is currently uncertain which tumors will be amenable to therapeutic intervention based on manipulating BM-derived endothelial precursor cells. The possibility that Hsp-function is critically involved in regulating endothelial cell differentiation from BM precursor cells to mature endothelial cells, perhaps by promoting endothelial cell survival to stress inducers (heat, IR), is a particularly interesting hypothesis. Finally, the prevailing paradigm is that intrinsic radiosensitivity of tumor cells is a major determinant of the radiation response of tumors, with limited contribution of extrinsic factors such as tumor microenvironment and host immune response. The experiments in this and the second aim of this proposal will provide a better understanding of the potential for targeting both parameters (microvascular endothelium and tumor cells) in tumor therapy.

### Example 7

### Hsp70i Deficiency Suppresses Den-Induced HCC Development

Encouraged by the above results with Hsf1−/− mice, we will determine the role of hsp70i in inflammation-associated liver tumorigenesis.

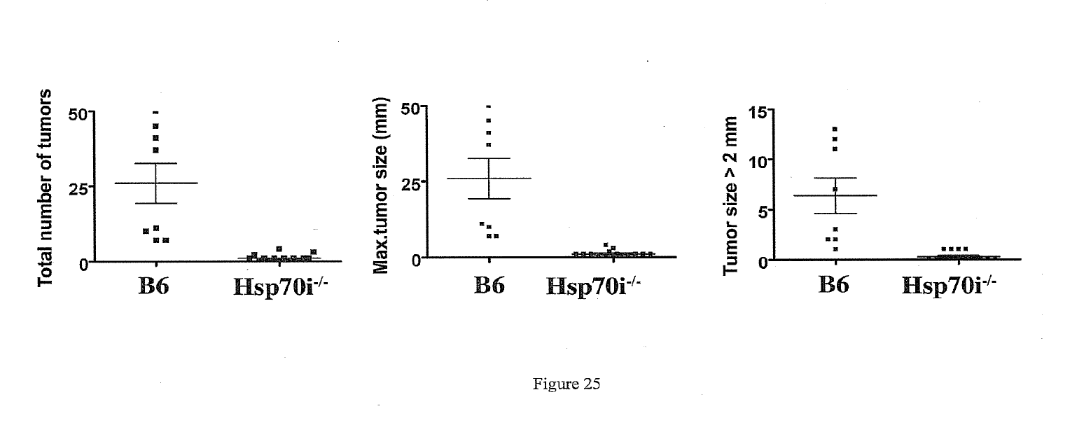

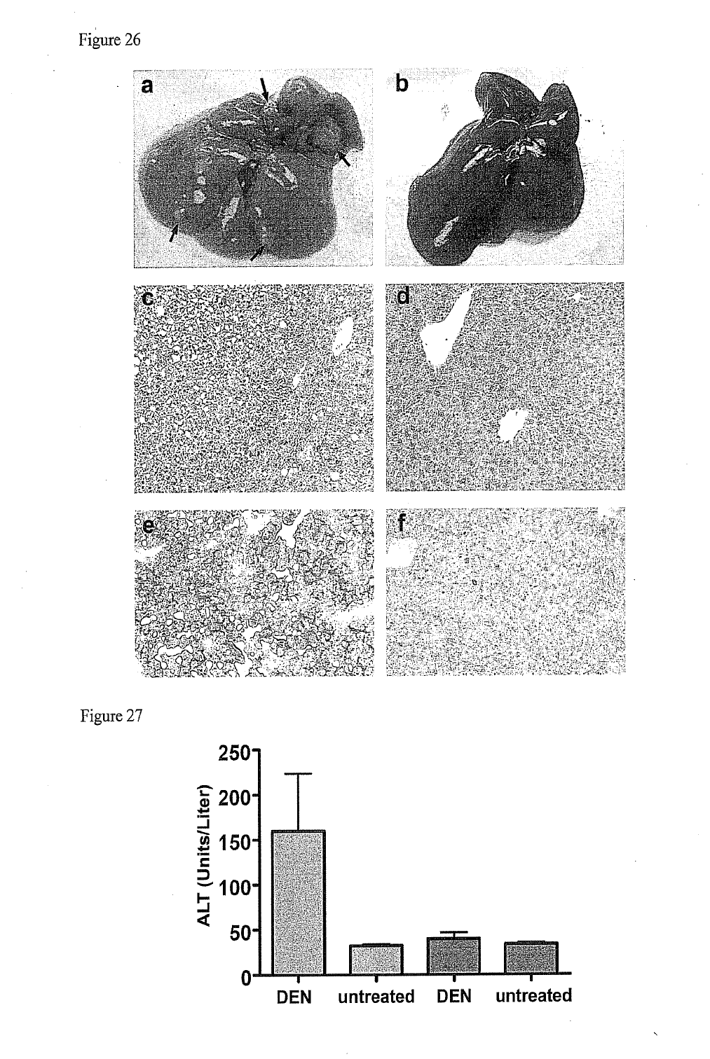

Long-term effects of DEN treatment. Hsp70i^{-/-} or hsp70i^{+/+} on a pure B6-genetic background were injected with DEN, and the tumor burden (number and size) was determined 7 months later (FIG. 25). Similarly to Hsf1^{-/-} mice, we found that ablation of Hsp70i protects from liver carcinogenesis, and the mice were almost free of tumors. Livers (with tumors) were photographed, fixed, sectioned, and stained with H&E (FIG. 26). Note the presence of HCC in the hsp70i+/+ mice (arrows). The majority of hsp70i−/− mice were free of tumors. In addition, histological examination of livers from Hsp70i−/− mice revealed a normal morphology. In contrast, DEN-treated B6 mice exhibited signs of persistent pathology associated with extensive destruction of hepatocytes and steatosis (lipid deposition). DEN administration is associated with DNA damage, apoptosis, necrosis, and cytokine production, which promote a compensatory proliferation of hepatocytes that leads to HCC development. To assess the liver integrity in precancerous tissue, we measured the circulating level of ALT in hsp70^{-/-} mice compared to hsp70i^{+/+} controls (FIG. 27). The data revealed high levels of ALT in wild-type mice bearing tumors, but only modest (borderline) enzyme levels in hsp70i^{-/-} mice.

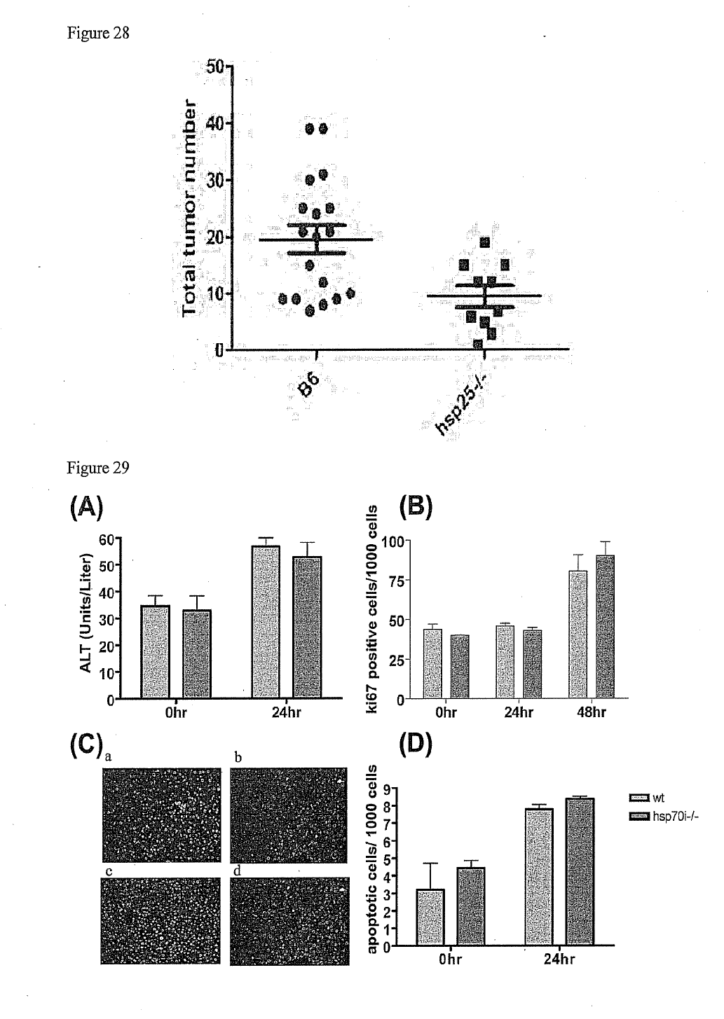

Hsp25 deficiency inhibits DEN-induced liver tumorigenesis. In further analyses we have evaluated whether Hsp25-mediated responses may play causal, supportive, or inhibitory roles in DEN-induced tumorigenesis. Ablation of Hsp25 also inhibits DEN-induced liver tumor formation, but in general the inhibitory effect was less dramatic compared to Hsp70i−/− or hsf1−/− mice (FIG. 28).

Hsp70i^{-/-} mice exhibit liver damage early after DEN administration, which is comparable to B6-controls. Short-teem effects of DEN on liver integrity are often good predictors of the outcome of the disease. We therefore measured the number of proliferating cells, level of apoptosis, and serum ALT levels (FIG. 29) 24-48 hours following DEN administration. FIG. 29 shows that the early response to DEN-induced liver damage, which is associated with cell death and cell proliferation, was comparable between Hsp70i^{-/-} and hsp70i^{-/-} mice. In addition, no significant differences in ALT levels were observed between the genotypes.

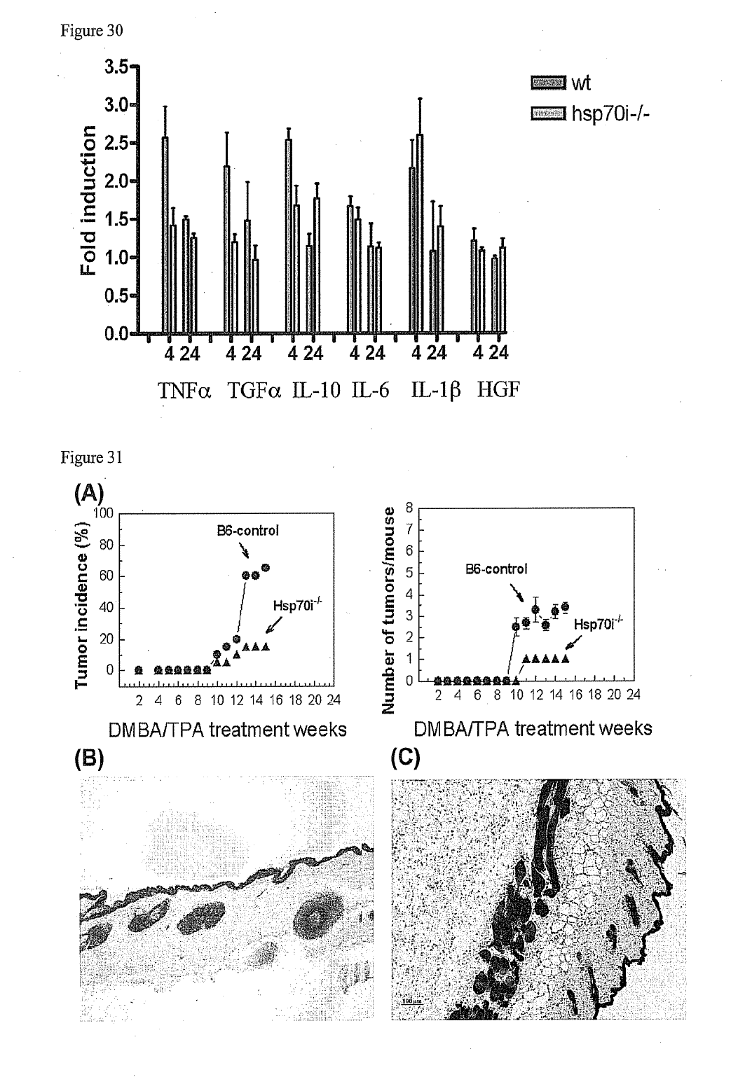

Effects of Hsp70i deficiency on cytokine production following DEN administration. DEN administration leads to increases in the levels of specific cytokines such as TNF-α, IL1β, IL-6, and TGFα as well as a number of growth factors such as hepatocyte growth factor (HGF). We have performed RT-PCR and cytokine arrays to compare the cytokine levels between wild-type and hsp70i−/− mice following DEN administration. The results revealed that the level of TNFα and IL1β and several other cytokines were comparable between wild-type and hsp70i−/− livers (FIG. 30).

### Example 8

### Hsp70i Deficiency Inhibits TMBA/TPA-Induced Skin Tumorigenesis

In further analyses we have evaluated whether Hsp70-mediated responses may play causal, supportive, or inhibitory roles in TMBA/TPA-induced skin tumors. Note that Hsp25 and Hsp70i are highly expressed in the epithelial layer of the skin. Although our initial study comparing Hsp70i-deficient mice with B6 control mice is not yet completed, the data so far, based on a 15-week observation period of a cohort of 10 Hsp70i−/− mice, suggest that loss of Hsp70i may be associated with a reduction in skin tumor incidence, as previously reported for Hsf1^{-/-} mice (FIG. 31). Collectively, the above findings with Hsf1^{-/-}, Hsp70i and Hsp25^{-/-} mice support the general tenet of our proposal that Hsp70i, Hsc70, and Hsp25 are essential in promoting inflammation-associated cancer and therefore are potential targets for cancer prevention in chronic inflammatory diseases.

### Example 9

### Impact and Mechanisms Underlying Hsp70, Hsc70, or Hsp25 Regulation of Chemical-Induced Liver Tumorigenesis

We will determine the intrinsic and extrinsic factors involved in DEN-induced HCC when Hsp70i, Hsp25, or Hsc70 is deleted from the whole organism, or when these Hsps (Hsp25 or Hsc70) are deleted from specific cell types (e.g., hepatocytes or macrophages). Note that since Hsc70 is essential for embryonic development we plan to use Hsc70+/− mice in our study, in which expression levels is substantially reduced (50-60%), compared to that in wild-type mice.

Whereas somatic mutations are often involved in tumor initiation, the mechanisms underlying tumor promotion are less well understood and are most likely associated with epigenetic factors. Increasing evidence suggests that exposure to carcinogens and chronic inflammation are two important mechanisms that lead to tumorigenesis, accounting for 20% of human cancers (Hanahan and Weinberg. 2000 Cell 100:57-70; Giaccia and Kastan, 1998 Genes Dev 12:2973-2983; Balkwill et al., 2005 Cancer Cell 7:211-217). In humans, HCC develops under the conditions of chronic hepatitis and cirrhosis, conditions in which hepatocytes are killed and liver inflammatory cells (macrophage-derived Kupfer cells) as well as newly recruited inflammatory cells (macrophages, NK cells, and neutrophils) are activated, driving proliferation of the surviving hepatocytes. TNF-α, TL-1b, IL-6, and the cytokine-induced chemokines produced by Kupfer cells (and other inflammatory cells) are key molecules involved in inflammatory reactions following administration of DEN (Karin and Greten, 2005 Nat Rev Immunol 5:749-759). Hsps are expressed in the liver following exposure to toxic stimuli such as DEN. Release of Hsps from necrotic cells can trigger activation of adjacent inflammatory cells (e.g., macrophages and neutrophils) that provide the pre-malignant cells with essential growth and angiogenic factors and thus may be beneficial for tumor initiation and promotion.

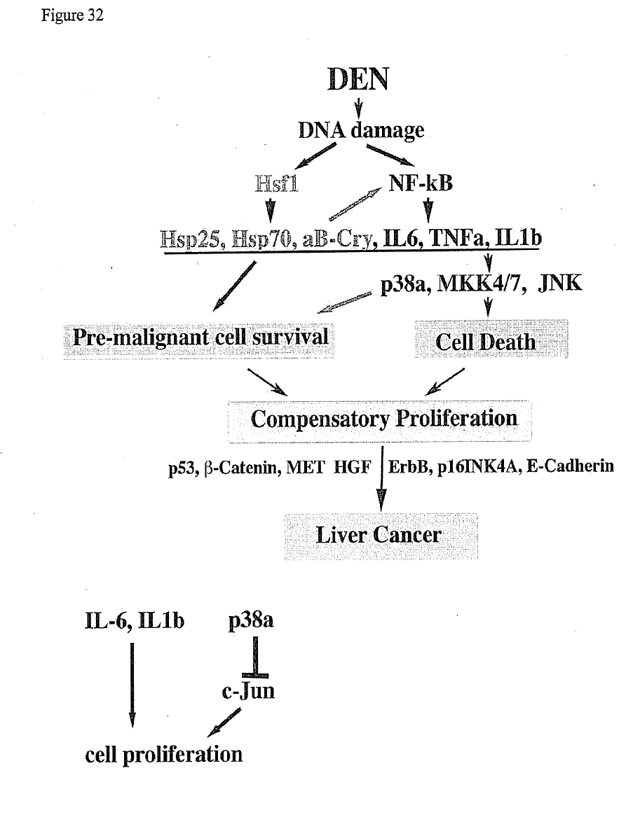

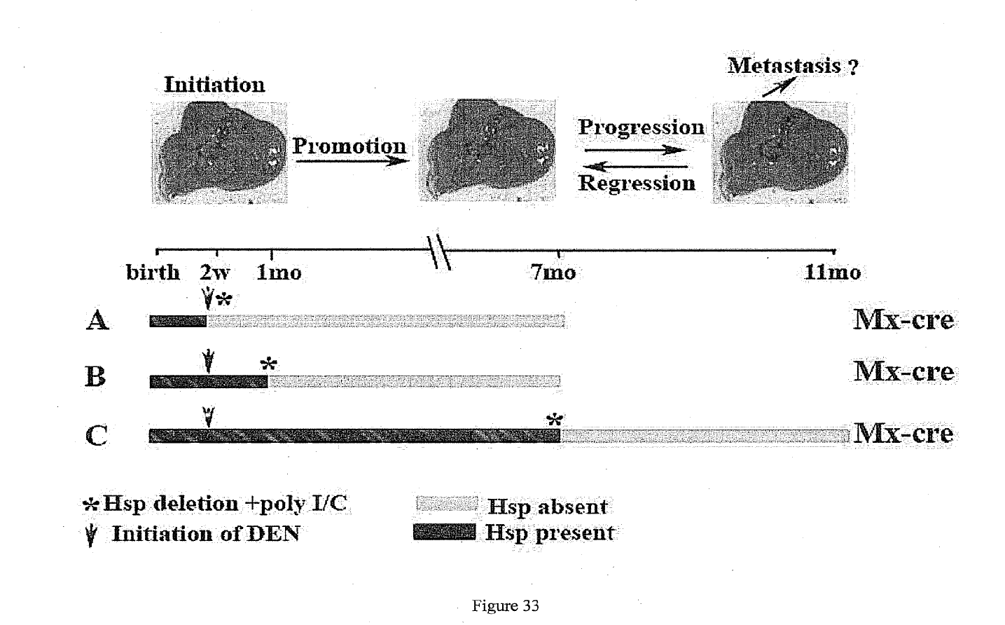

Experiments proposed in this example will determine if Hsp70i, Hsc70, and Hsp25 chaperones are crucial for chemically induced HCC, and their disruption in vivo may inhibit tumor growth. The specific premise to be tested is that deletion of these Hsps individually or in combination will decrease premalignant cell survival based on their function as potent negative regulators of apoptosis induced by stress stimuli and positive regulators of inflammation and sustained cytokine production. Our recent studies have revealed that initial liver damage and inflammatory response following DEN treatment are indistinguishable between Hsp70i−/− mice and wild-type controls. Based on this information, it is likely that although Hsps are expressed (or induced) early in the course of disease, they are dispensable for a pronged period, during time, which pre-malignant cells accumulate in the inflamed liver, but they are critical at a late stage at which these cells become malignant. Acquisition of oncogenic mutations during the tumor initiation stage renders pre-malignant cells sensitive to apoptosis, which may be prevented by Hsps. FIG. 32 shows a potential role for Hsps in DEN-induced HCC. DEN administration causes DNA damage, which is associated with the initiation or death of hepatocytes through both apoptosis and necrosis. This leads to activation of NF-κB (Sakurai et al., 2008 Cancer Cell 14:156-165; Sakurai et al., 2006 PNAS 103:10544-10551) and enhanced production of inflammatory cytokines. Inflammatory signal provides apoptosis protection for proliferating hepatocytes in response to liver damage. Acquisition of oncogenic mutations accumulated during the tumor promotion phase renders premalignant hepatocytes more vulnerable to apoptotic factors. Hsp-mediated cell protection (anti-apoptotic function) becomes more critical, especially at the stage when these cells turn malignant. In addition, Hsp release from necrotic cells can activate further the inflammatory process, perpetuating the cycle of necrosis, apoptosis, and proliferation. This stimulates the proliferation of surviving hepatocytes with mutations in genes, such as p53, MET receptor, and its ligand, hepatocyte growth factor (HGF), which finally leads to HCC development (Karin and Greten, 2005 Nat Rev Immunol 5:749-759; Farazi and DePinho, 2006 Nat Rev Cancer 6:674-687). Thus, ablation of Hsp70i, Hsc70, or Hsp25 negatively impacts cell transformation and HCC tumor progression largely based on their property to inhibit cell apoptosis and regulate components of the inflammatory response in the tumor environment.

We will study the roles of Hsp70i, Hsc70 and Hsp25 in DEN-induced HCC development. In order to assess the therapeutic potential of Hsp-inhibition in tumor regression we will perform experiments of Hsp25 or Hsc70 ablation in established DEN-derived tumors using an inducible Cre-lox deletion strategy. Note that the Hsp70i mouse line with a conditional loxP-flanked allele is not currently available, thus preventing us from performing similar experiments of inducible Hsp70i ablation. The generation of a Hsp70 conditional mouse is technically very challenging because the distance between the loxp sites is large (approximately 14 kb). However, theoretically it is possible to generate a such mouse model, by isolating ES clones with a loxp site inserted in front of the Hsp70.3 gene and in a second step we could be able to introduce the other loxp site at the end of the Hsp70.1 gene. This is not a easy task and the chance to isolate ES clones using gene targeting approaches with recombination events outside of the 14 kb deletion fragment is very low.

Experiment 1 will test the role of Hsp25, Hsc70, or Hsp70i in evolution of DEN-induced HCC. HCC is the most common liver cancer in humans, with distinct prevalence in men (Farazi and DePinho, 2006 Nat Rev Cancer 6:674-687). In DEN-induced HCC, male mice are also 3-5-fold more susceptible to HCC development than female mice. Estrogen, which is present in females but not males, suppresses IL-6 production following DEN administration and has been proposed as a potential mechanism for the enhanced resistance of female mice to HCC (Karin and Greten, 2005 Nat Rev Immunol 5:749-759; Farazi and DePinho, 2006 Nat Rev Cancer 6:674-687; Maeda et al., 2005 Cell 121:977-990; Naugler et al., 2007 Science 317:121-124). In this model system, 100% of the wild-type male mice develop HCC within 7 months following DEN administration (Maeda et al., 2005 Cell 121:977-990). In initial studies we will determine whether Hsp70i or Hsp25 deficiency or Hsc70 haploinsufficiency affects DEN-induced HCC development. To this effect, Hsp25−/−, Hsp70i−/−, or HSC70+/− mice (male) at 15 days of age will be injected with a single dose of 25 mg/kg DEN. The animals will be observed for HCC development over a period of 7 months (or up to 2 years, if necessary). Analyses will be performed, as described in detail below and demonstrated in FIG. 29, to determine the short-term effects of DEN administration, which include cytokine production and increased hepatocyte apoptosis, necrosis, and compensatory proliferation (Sakurai et al., 2008 Cancer Cell 14:156-165; Sakurai et al., 2006 PNAS 103:10544-10551; Naugler et al., 2007 Science 317:121-124), and long-term effects, which include accumulation of genomic mutations and development of HCC (Farazi and DePinho, 2006 Nat Rev Cancer 6:674-687).

Exp. 1.a is a study of the short-term effects (initiation phase) of DEN-induced HCC: Short-term effects (immediate acute) of DEN on liver cells can predict the outcome of liver disease. Thus, a strong increased level of specific cytokine production, increased liver cell apoptosis and necrosis, increased compensatory cellular proliferation, and high level of liver enzyme concentrations such as ALT in serum measured within the first 4 to 48 hrs following DEN administration, are all indicative parameters for enhanced sensitivity of mice to DEN-induced HCC. To examine the immediate (acute) effects of DEN administration, a cohort of 15 Hsp-deficient mice and B6 controls will be injected with DEN, and at 0 (untreated control), 4, 24, and 48 hrs, mice will be euthanized and analyzed as below.

In Exp. 1a-1, an indicator for liver damage following DEN administration, circulating liver enzyme (ALT) activity will be measured using a kit (Pointe Scientific, Inc.). The cytotoxic effects of DEN are dependent on its metabolic activation within the hepatocytes by cytochrome P450 2E1 (CYP 2E1) (Weber et al., 2003 Crit. Rev Toxicol 33:105). To examine whether DEN metabolism may account for the difference in HCC development, CYP2E1 mRNA will be measured by real-time PCR at 4, 24 and 48 hours. Liver cell death will be determined by TUNEL assay on tissue sections, and apoptotic cells will be counted. In addition, liver tissue sections will be stained with H&E, and size of necrotic areas will be quantitated using MetaMorph software (Maeda et al., 2005 Cell 121:977-990). To quantitate the proliferative response following DEN administration, mice will be injected with BrdU two hours later, livers will be collected, and sections will be stained with antibody to BrdU. The number of proliferating cells will be quantitated.

In Exp. 1a-2, expression of cytokines and growth factors, including IL6, TNF-a, TGF-a, IL1b, and HGF, will be determined by profiling cytokine mRNA expression (SuperArray, Bioscience Co) or RT-PCR in liver and by measuring cytokines in serum. To determine the cellular source of inflammatory factors, we will determine the level of cytokines (IL-6, IL1b) in supernatants of cultured hepatocytes (isolated using a fractionated scheme that separates hepatocytes and macrophages (Kupfer cells)) following their stimulation with TNF-a (20 ng/ml plus D-galactosamine (5 mM) for 0, 15 min, 30 min, and 1, 3, 6 and 12 hrs) (Eferl et al., 2003 Cell 112:181-192). For more comprehensive analysis, we will perform cytokine arrays that measure mRNA expression levels of 114 common cytokines. TNFa-treated hepatocyte cultures will also be used to determine the expression of Hsps by immunoblotting.

In Exp. 1a-3, as NF-κB activation plays a major role in inflammation-associated HCC development, nuclear extracts prepared from livers will be subjected to EMSA to detect NF-κB DNA binding activity. In addition, we will measure the level of phosphorylated IKKa (and IKKb and NF-κB (phospho-p65)) by immunoblotting.

To determine expression of activated NF-κB target genes in livers of mice treated with DEN (0-24 hrs), we will profile mRNA levels using NF-κB arrays (SABiosciecnes) that detect a number of transcription factors such as Atf1, Atf2, Crebbp, Egr1, Elk1, Fos, Ifng, Irf1, Jun, Nfkb2, Pcaf, Rel, Rela, Relb, Smad3, and Stat1 and inflammatory response genes such as Myd88, TLR1, 2, 3, 4, 6; IKK1, IKK2, CSF 2, 3; 1RF1, INFg Bcl3, Casp1, Casp8, and Fadd. We will also perform analyses to measure expression of molecules of other signaling pathways involved in inflammatory responses, such as the JAK/STAT pathway. This array detects mRNA expression of 84 genes including STAT1-6, JAK1-3, Ifng, Irf1, Jun, Smad2, Smad3, Smad4, Nfkb1, Sla2, and Smad3. These analyses are critical to assess whether the observed or predicted enhanced resistance of Hsp-deficient mice to HCC is associated with impaired NF-κB and JAK/STAT signaling.

In Exp. 1a-4, expression of proteins involved in the stress response and in proliferation, as well as of known cell cycle regulators, tumor suppressors, and anti-apoptotic molecules, will be determined by immunoblotting of liver extracts. In particular this analysis will include detection of Hsf1, p19Arf, STAT3, phospho-STAT3 (downstream target of IL-6), VEGF, c-Jun, phospho-JNK1 (sustained activation has been shown early after DEN administration) (Farazi and DePinho, 2006 Nat Rev Cancer 6:674-687; Giaccia and Kastan, 1998 Genes Dev 12:2973-2983), cyclin D1, p53, and p53 target genes (P21cip1, PUMA, NOXA, and Bax), as well as Bcl-2 and Bcl-XL, and Hsps (-90, -70, -25, αB-Crys, etc.).

Exp. 1a-5 will assess the accumulation of superoxide anions, liver tissue sections will be stained with dihydroethidine (or using antibody to 8-hydroxydeoxyguanosine (8-OHdG)) (Eferl et al., 2003 Cell 112:181-192) and the intensity of fluorescence will be quantitated. In addition, CHOP has been shown to be a ROS-induced regulator of apoptosis, and its expression is enhanced by a number of stressors (Sakurai et al., 2008 Cancer Cell 14:156-165). Expression of CHOP will be determined by immunoblotting. Increased production of ROS is the major contributor to JNK activation and acute liver failure. Hsps are important protectors against oxidative damage and are induced upon oxidative stress (Arrigo, 2007 Adv Exp Med Biol 594:14-26).

In Exp. 1a-6, the levels of reduced glutathione (GSH), a major cellular anti-oxidant, will also be determined in liver extracts of untreated controls (O), or at 4, 24, and 48 hrs following DEN injection (Eferl et al., 2003 Cell 112:181-192; Maeda et al., 2005 Cell 121:977-990). Note that reduced levels of GSH correlates with increased incidence in DEN-induced HCC. Additionally, DEN-induced lipid peroxidation will be determined using malondialdehyde (MDA) and 4-hydroxyalkenals (HAE). Increase in lipid peroxydation is associated with enhanced liver tumorigenesis (Eferl et al., 2003 Cell 112:181-192).

In Exp. 1b, long-term effects (progression phase) of DEN-induced HCC will be determined. As mentioned before, DEN-induced HCC appears at 5-7 months following DEN administration in wild-type mice. To determine the impact of DEN on liver tumorigenesis in wild-type or Hsp-deficient mice, we will determine the following parameters at the termination of our experiments: mouse weight, liver weight, number of tumors per liver (% tumor incidence), maximal tumor size (mm), and % tumor area/liver. To examine the effects of DEN on cell behavior and liver integrity during the progression phase (which involves an increase in size and spreading of tumor), liver/nontumor tissue sections will be H&E stained and examined for pathological alterations. Tumor types (adenomas or carcinomas) as well number of HCCs in individual mice will be determined according to the criteria established by World Health Organization (WHO) and in the literature (Eferl et al., 2003 Cell 112:181-192; Sakurai et al., 2008 Cancer Cell 14:156-165; Maeda et al., 2005 Cell 121:977-990; Sakurai et al., 2006 PNAS 103:10544-10551). HCCs often express afetoprotein, and immunostaining of tumor/non-tumor liver tissue sections using antibody to this protein is a good marker to identify HCC regions in liver sections. In addition, to measure the extent of cell proliferation, mice will be injected with BrdU and liver sections will be stained with antibody to BrdU. Liver/tumor sections will be immunostained with antibody to CD31 or CD11b to detect and quantitate the number of blood vessels and the degree of inflammatory cell infiltration, respectively (Maeda et al., 2005 Cell 121:977-990).

Tumors and non-tumor liver tissues will also be extracted for immunoblotting or for mRNA gene profiling analyses. As noted for short-term DEN exposure analyses, the expression of proteins involved in tumorigenesis and stress response, such as Hsf1, p19Arf, phospho-STAT3, STAT3, VEGF, NF-κB, IKKb, phospho-JNK1, cyclin D, c-myc, p27kip1, p53, and p53 target genes such as p21, PUMA, NOXA, and Bax, will be determined. Additional analyses will determine expression of the anti-apoptotic proteins Bcl-2 and BclxL and major Hsps (-90a & b, -70, -60, -40, -25, aB-crys, and Hsc70). For each mouse analysis, lungs will be removed and examined macroscopically or histologically for tumor metastasis (human-like HCC often metastasizes to the lung).

Experiment 2 will examine the role of Hsp25, Hsc70, or Hsp70i in HCC development induced by a single administration of DEN in combination with tumor promoter treatment. Administration of DEN to B6 mice (male) at 15 days of age leads to induction of HCC. However, this experimental design does not allow us to assess the role of Hsps in tumor promotion and progression in the adult. The rationale to study liver tumorigenesis in the adult is also provided by the fact that liver cancer is an aging disease in humans and is caused by chronic hepatitis and liver damage. DEN, once activated by cytochrome p450 enzymes, generates alkylating agents that modify DNA and induce mutations (Liu and Waalkes. 2008 Toxicol Sci 105:24-32).

However, in adult mice, DEN administration is not sufficient for liver tumorigenesis due to reduced toxicity of DEN, which is a weak carcinogen and requires assistance from tumor promoters. Tumor promoters such as phenobarbital apparently act through not yet known mechanisms and eventually induce hepatocyte proliferation, which transmits oncogenic mutations to their progeny, leading to HCC (Eferl et al., 2003 Cell 112:181-192). To address the above issue we will analyze the function of Hsps in liver cancer development using the DEN-phenobarbital protocol. Tumors will be initiated by injection of DEN at 4 or 8 weeks of age (older mice have a much reduced response to DEN-induced HCC) and promoted with a diet (Sniff) containing 0.07% Phenobarbital (Sigma) started at 8 or 12 weeks of age, correspondingly, until mice are sacrificed.

In the design of this experiment we will emphasize targeted analyses as described above (see design of Exp. 1, above). We will determine the immediate early effects of DEN administration associated with liver damage (circulating liver enzyme concentrations of (ALT, GOT, and GPT), TUNEL assay and quantitation of necrotic areas), or DEN metabolism measuring CYP2E1 mRNA level by real-time PCR at 4, 24, and 48 hours. In addition, progression of HCC will be evaluated by determining the survival rates of mice at 9-12 months age, mouse weight, liver weight, number of tumors per liver (% tumor incidence), maximal tumor size (mm), and % tumor area/liver. In addition, tumor types (adenomas or carcinomas) as well as number of HCCs in individual mice will be determined following the criteria established by WHO and in the literature.

In Experiment 3, conditional deletion of Hsp25 or Hsc70 from hepatocytes or inflammatory cells during HCC development will be used to examine the role of Hsps in tumor initiation, promotion, and progression. To determine whether and at which stages (tumor initiation, tumor promotion, and tumor progression) Hsp25 or Hsc70 is required for survival of pre-malignant and malignant liver cells, we will perform dedicated tumor regression experiments by inducing Hsp25 or Hsc70 gene ablation at different times after tumor initiation. This analysis will allow us to investigate whether Hsp25 or Hsc70 inhibition is sufficient to induce regression of HCC, which is generally refractory to clinical treatment.

First, we will establish mice carrying floxed alleles of Hsp25 or Hsc70 and the Mx-cre transgene (Mx1-hsp25flox/flox or Mx1-Hsc70flox/flox). Expression of the Mx-Cre transgene can be induced by injection of poly I/C, which according to the literature leads to almost complete deletion of the gene of interest in liver tissue (hepatocytes and non-parenchymal cells). However, we need to verify this for our experiments by performing Southern blot analysis and genomic PCR. Development of tumors will be initiated at 2 weeks of age by a single injection of DEN, and the hsp25 or hsc70 gene will be deleted by injection of PolyI/C (indicated by *) at different times (tumor initiation at 2 weeks of age, transition phase at 30 days of age, or tumor progression phase at 7 months of age) during the course of the disease (see also experimental protocol in FIG. 29). In studies where tumor regression, for example by Hsp-ablation during the tumor progression phase (at 7 months of age), will be examined, we will use the Small Animal Magnetic Resonance Imaging (MRI) facility at MCG to image tumors that arise. In this case, MRI will be a valuable tool to determine the initial tumor size and whether ablation of Hsps leads to a decrease in tumor progression. The Small Animal MRI facility at MCG operates on a fee-for-service basis. Tumor development will be monitored using MRI within a 4-month period (1, 2, and 4 months after gene ablation). Liver tumor volume will be calculated by analyzing all coronal and axial images of each animal. Histological analysis of liver tissue taken at the end of each treatment regime will be performed to examine liver morphology with respect to the presence of differentiated tumor cells that have lost their characteristic neoplastic histological features (high mitotic index, large nucleoli, high nuclear/cytoplasmic ratio) and increased apoptosis of transformed hepatocytes (TUNEL assay) and inflammatory cells. Tumor regression is associated with differentiation of tumor cells and eventually most of the cells undergo death.

The analyses proposed in this aim will provide us with conclusive answers about the contribution of the molecular machinery consisting of the core Hsp70/Hsc70 complex in carcinogen-induced HCC formation. In addition it will generate critical information about the role in this disease of Hsp25, which shares many functional features with the Hsp70s. In support of this proposal, our study has demonstrated that Hsp70i ablation from the whole body renders mice resistant to HCC development induced by DEN. In addition, Hsp25 ablation in this model also inhibits HCC development. The design of Exp. 1 is to extend these observations to include analyses on Hsc70+/− mice and examine the mechanism(s) by which Hsp inactivation may cause HCC inhibition.

The prevailing paradigm in the literature suggests that cell necrosis and apoptosis and compensatory proliferation induced by DEN administration, especially in the acute phase, have a critical role in HCC development. Therefore analyses described in this experiment to determine the short-term effects (immediated/acute) of DEN on liver cells can generate data to predict the course and perhaps the outcome of this liver disease. Interestingly, data presented in Examples 4 and 5 showed that although DEN-administration rapidly induced liver cell necrosis apoptosis, cell proliferation, and production of cytokines, the response pattern between Hsp70i−/− mice versus B6 controls were comparable. This indicates that the tumor promoting function of Hsp70i may be more critical during the promotion and progression phase than the initiation phase of HCC development. The analyses proposed to determine the long-term effects (progression phase) of DEN-induced HCC should provide us with more conclusive data to address this aspect. Furthermore, studies with Hsp25−/− or Hsc70+/− mice will be very informative to assess their potential role in modulating the tumorigenesis process.

How do Hsps support tumor promotion? Hsps have a major anti-apoptotic effect and are necessary to protect mature hepatocytes from genotoxic stress. Compromising the Hsp function, especially in pre-malignant cells, could contribute to the tumor suppressive effects of the Hsp ablation in the Hsp70i−/− mice. Additional mechanisms by which Hsp ablation may inhibit tumor progression can involve the NF-κB, JNK, and STAT3 activation pathways. In particular, mice lacking JNK1 are much less susceptible to DEN-induced hepatocarcinogenesis. This impaired tumorigenesis correlated with decreased expression of cyclin D and vascular endothelial growth factor, diminished cell proliferation, and reduced tumor neovascularization. Furthermore, sustained NF-κB and STA3 activation pathways have been shown to be critical for oncogenic progression (Yu et al., 2007 Nat Rev Immunol 7:41-51; Kortylewski et al., 2009 Cancer Cell 15:114-123). The detailed analyses proposed above will evaluate whether defective activation of these pathways in the absence of Hsps can explain the effects on HCC progression.

Another important aspect under investigation in this specific aim (Exp. 3) is to examine the possibility that Hsp inactivation may be sufficient to induced sustained tumor regression or even eradication. If it turns out to be true, this will indicate that non-oncogene inactivation such as Hsp ablation in a liver tumor, which is particularly refractory to therapeutic intervention, would be effective to treat this tumor. One exciting avenue that will be considered as a future direction will be to determine whether the enhanced resistance to HCC by Hsp inactivation reflects reduced malignant expansion and survival of immature liver cells with stem-like features. Many recent studies report that cancers frequently consist of cellular subpopulations, some of which have retained stem cell properties and are derived from these cells. HCC tumor stem cells in humans appear to be CD45−CD95+ (Yang et al., 2008 Cancer Cell 13:153-166). However, whether these surface markers define liver tumor stem cells in mice needs to be clarified. If this can be confirmed, additional experiments will be performed to determine whether Hsp-inactivation during the progression phase of tumor development may lead to differentiation and complete elimination by cell death of this stem cell population.

Statistical analyses. Data will be expressed as mean+/−SEM. Differences will be analyzed by Student's t test. p values less or equal to 0.05 will be considered significant. In all cases group sizes will be selected to generate results to ensure there is no ambiguity in the statistical analyses.

### Example 10

Impact and Mechanisms Underlying Hsp70, Hsc70 or Hsp25 Regulation of Chemical-Induced Skin Tumorigenesis

Hsp70i, Hsp25, and Hsc70 are highly expressed in the epithelial layer of the skin including hair follicles where the precursors for epithelial regeneration reside. Recent evidence in the literature reveals that hsf1 ablation in a mouse model for inflammation-associated skin tumorigenesis significantly reduces the incidence of tumors. In addition, our ongoing study suggests that Hsp70i ablation negatively impacts skin tumor development in this model. In this example, we will examine if ablation of Hsp70i, Hsc70, or Hsp25 negatively impacts the skin tumor progression based on their properties to regulate survival of premalignant cells and thus cell transformation as well as components of the inflammatory response in the tumor environment. A specific premise to be tested is that Hsp expression is required to maintain skin tumorigenesis, and ablation of Hsps from established skin tumors will result in their regression or even eradication.

Experiment 4 will test the role of Hsp25, Hsc70, or Hsp70i in chemical skin carcinogenesis in mice. To investigate the role of Hsp25, Hsp70i, or Hsc70 as a modifier of epithelial cell tumorigenesis, we will use a classical multistep chemical skin carcinogenesis protocol. This protocol entails tumor initiation in epidermal keratinocytes by a single treatment with the carcinogen 7,12-dimethylbenzanthracene (DMBA), which invariably induces oncogenic activation of the c-Ha-Ras gene (Ambler and Maatta. 2009. J Pathol 217:206-216). Subsequent repeated treatments with the tumor promoter 12-O-tetradecanoylphorbol-13-acetate (TPA) result in the outgrowth and progression of initiated cells. Benign tumors (largely papillomas) appeared in B6 mice within 7-10 weeks from DMBA administration.

A cohort of 15 male or female Hsp25−/−, Hsp70i−/−, Hsc70+/− mice and B6 controls, 8 weeks old, will be treated with DMBA (100 μg applied topically to each mouse). For tumor promotion, TPA will be applied twice a week for 25 weeks. Initiation and progression of skin tumors will be studied as follows:

Skin tumor formation will be monitored weekly and the incidence and latency to tumor development and lethality will be determined over the period of the experiment (50 weeks). In addition, tumor burden (number and size of tumors) will be recorded. Tumor dimensions will be measured by caliper, and the tumor volume will be calculated as [(width)2×length]/2.

Histology of mouse skin stained with H&E will be performed for microscopic analysis of skin lesions and tumor type characterization and progression. Skin tumors that arise using the DMBA/TPA protocol comprise a spectrum of histopathological types; they are usually benign, such as papillomas and keratoacantomas, or can exhibit a malignant phenotype (complex lesions with focal invasion, squamous cell carcinoma, or spindle cell carcinoma). For further characterization of skin tumors, immunohistological analysis will be performed to visualize Hsp expression (staining for reporter LacZ) combined with staining for the mesenchymal marker vimentin and keratinocyte markers (keratin 1 and keratin 14) (Malliri et al., 2002 Nature 417:867-871).

Short-term effects. Apoptosis in skin 24 hours following application of DMBA or TPA or both to the back of mice of each genotype will be detected by TUNEL assay to assess whether Hsp deficiency renders cells more susceptible to this treatment regimen. This may explain the predicted resistance of Hsp mutant mice to tumor initiation. In addition, we will detect proliferating cells in the basal layer of the epidermis following treatment of mice with DMBA or TPA for 24 hours or DMBA and subsequent treatment with 2 doses of TPA over 7 days by injection of BrdU 2 hours before the end of the experiment. The number of BrdU+-epidermal keratinocytes in the basal layer of interfollicular regions from several independent fields will be evaluated per 100 basal keratinocytes. Additional analyses will evaluate the extent of the inflammation in the hyperplastic epidermis following chemical treatment. To this effect, skin sections from Hsp-deficient and B6 controls will be stained for macrophages (F4/80), T cells (CD3), or B (CD45R) cells and analyzed microscopically. Further analyses in vitro with cultured keratinocytes derived from different genotypes will assess the effects of Hsp ablation on cell proliferation and differentiation, cytokine production following mitogene stimulation (TPA), as well as induction and activation of NF-kB and c-JNK signaling pathways (see also design of Specific Aim 1). Mouse primary keratinocytes will be isolated and cultured as described in the literature (Hu et al., 2001 Nature 410:710-714; Liu et al., 2008 Cancer Cell 14:212-225).

To determine whether Hsp ablation affects the cell's ability to acquire Ras mutations, DNA from skin tumors will be extracted and sequences flanking the 61st codon will be amplified by nested PCR as described previously (Finch et al., 1996 Carcinogenesis 17:2551-2557). However, this analysis is feasible assuming that Hsp ablation does not completely inhibit skin tumor development. As an independent in vitro assay, we will compare the susceptibility of MEFs derived from Hsp-deficient mice versus B6 controls to transformation induced by mutated H-Ras or Myc in a focus formation assay. In this assay, MEFs of different genotypes will be transduced with retroviruses encoding green fluorescent protein (GFP), mouse Hsp or oncogenic H-RasV12D (a Ras mutation commonly found in human cancers), Myc or H-RasV12D and Myc. After 2-3 weeks cells will be fixed in methanol and foci visualized by staining cells with 0.1% toluidine blue. The number of foci will be measured using CellProfiler software.

To further discriminate between the effects of Hsps deficiency on DMBA-induced tumor initiation versus TPA-induced tumor promotion, a cohort of 15 mice will be repeatedly treated with DMBA alone (5 μg twice a week for 25 weeks), rather than with DMBA and TPA. This complete carcinogenesis protocol leads directly to the formation of predominantly squamous cell carcinomas.

In Experiment 5, conditional deletion of Hsp25 or Hsc70 from hepatocytes or inflammatory cells during HCC development will be used to examine the role of Hsps in tumor initiation, promotion, and progression. To determine whether Hsp25 or Hsc70 is required for tumor progression, a condition that is similar to the clinical setting, we will perform dedicated tumor regression experiments by inducing Hsp25 or Hsc70 gene ablation in established DMBA/TPA-derived skin tumors (K14-creER^{T2}-Hsp25^{flox/flox} or Hsc70^{flox/flox}). First, we will establish mice carrying foxed alleles of Hsp25 or Hsc70 and the K14-creER^{T2}. For gene deletion the creER^{T2} will be induced by local application of tamoxifen (8 treatments over 8 days) to the shaved skin as described recently (Dhomen et al., 2009 Cancer Cell 15:294-303). This leads to almost complete gene deletion in the treated skin area. In an alternative treatment schedule, gene deletion will be achieved in the entire skin by injection of 1 mg of tamoxifen daily for five days. In both cases we will need to verify this by performing Southern blot analysis and genomic PCR.

The design of the experiment is to induce skin tumors by DMBA/TPA as described above. At the time where skin tumors are well developed (20 weeks of treatment) cre-mediated Hsp ablation will be induced by treatment of K14-creER^{T2}-Hsp25^{flox/flox} or K14-creER^{T2}-Hsc70^{flox/flox} mice will either vehicle alone (and serve as control), or with tamoxifen. The mice will be observed for an additional period of 20 weeks for tumor regression by determining the tumor burden (number and size of tumors) in mice with tamoxifen versus vehicle treatment. At the end of the experiment, histology of mouse skins stained with H&E will be performed for microscopic analysis of skin lesions with respect to cell morphology (terminal differentiation or mitotic figures) and extent of tumor regression. Additional analyses similar to those described above (see design of Exp.4 a, b and in Specific Aim 2) will be pursued as needed.

Epithelial-derived tumors are the most common form of human cancers (Farley et al., 2008 Cell Res 18:538-548; Bussolati et al., 2009 J Cell Mol Med 13:309-319). Mouse model systems that closely resemble human cancers are of great value since they provide a unique opportunity to analyze the mechanisms underlying tumor initiation and tumor regression. We anticipate that both studies outlined in Experiments 4 and 5, will provide compelling evidence for the involvement of Hsp70i, Hsc70, and Hsp25 in skin tumor initiation as well as their importance to sustain long-ter n tumor growth and malignancy.

How do Hsps support skin tumor promotion? A prominent function of Hsps under investigation in this proposal is their ability to protect cells from apoptotic death induced by genotoxic stress. Compromising Hsp function, especially in the pre-malignant stage, may affect the survival of keratinocyte-lineage-derived tumor cells and thus contribute to tumor suppressive effects in the Hsp-deficient mice. Interestingly, MEFs lacking Hsf1 are refractory to transformation induced by RASV12D and show markedly decreased proliferation following RASV12D transduction. In addition, Hsf1 depletion from established human tumor cell lines strongly impairs their growth and survival but have little effects on normal cell viability. Thus, it is tempting to propose that Hsp ablation in our study will not have significant effects on Ras mutations and tumor initiation induced by DMBA/TPA, but their function may be important for the proliferation and survival of pre-malignant cells with Ras mutations. An additional mechanism for pre-malignant cell survival is provided by inflammatory factors produced in the skin following DMBA/TPA treatment. Constitutive lisp expression in the skin may function as a promoter of the inflammatory response by regulating, for example, sustained NF-κB activation. Finally, recent reports described that within advanced tumors induced with the DMBA/TPA model a population of CD34+ cells seems to be of functional importance for skin tumor formation (Owens and Watt. 2003 Nat Rev Cancer 3:444-451; Ambler and Maatta. 2009 J Pathol 217:206-216). This cell population is identified in early and advanced skin tumors and has been proposed to represent cancer stem cells, which closely resemble normal bulge skin stem cells. Thus, an interesting avenue for further consideration in our proposal will be to investigate whether Hsp ablation may affect the survival and perhaps differentiation potential of this stem-cell-like cell population, which seems to be important for long-term tumor growth and perhaps initiation. The detailed analyses proposed above will evaluate all these aspects.

Statistical analyses. Data will be expressed as mean+/−SEM. Differences will be analyzed by Student's t test. p values less or equal to 0.05 will be considered significant. In all cases group size will be selected to produce results that are statistically unambiguous.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1. The genetically engineered non-human animal comprising an exogenous DNA of claim 12, wherein the exogenous DNA reduces or eliminates function of a molecular chaperone, and wherein the animal is predisposed to brain pathology.

2. The animal of claim 1 wherein brain pathology comprises a neurodegenerative disease, cognitive disorder, or traumatic brain injury.

3. The animal of claim 2 wherein neurodegenerative disease is selected from the group consisting amyotrophic lateral sclerosis, Alzheimer's disease, Parkinson's disease, and polyglutamine diseases.

4. The animal of claim 1, wherein the brain pathology comprises aggregation of Aβ and/or hyperphosphorylation p-tau.

5. The genetically engineered non-human animal comprising an exogenous DNA of claim 12, wherein the exogenous DNA reduces or eliminates function of a molecular chaperone, and wherein the animal is predisposed to reduced angiogenesis and/or tumorgenesis.

6. The animal of claim 5, wherein angiogenesis comprises tumor angiogenesis.

7. The animal of claim 5, wherein tumorgenesis comprises chemically induced tumorenesis.

8. The animal of claim 5, wherein tumorgenesis comprises tumor initiation and/or tumor growth.

9-10. (canceled)

11. The animal of claim 12 wherein the animal is a mouse.

12. A genetically engineered non-human animal comprising an exogenous DNA, wherein the animal demonstrates reduced or eliminated function of the heat shock protein 110 (Hsp110) and/or reduced or eliminated function of the heat shock protein 70 (Hsp70).

13. A cell isolated from a genetically engineered non-human animal comprising an exogenous DNA of claim 12.

14. A genetically engineered cell comprising an exogenous DNA wherein the exogenous DNA reduces or eliminates the function of heat shock protein 110 (Hsp110).

15. A genetically engineered cell comprising an exogenous DNA wherein the exogenous DNA reduces or eliminates the function of heat shock protein 70 (Hsp70).

16. The cell of claim 13, wherein the cell demonstrates a pathology of a neurodegenerative disease, cognitive disorder, or traumatic brain injury.

17. The cell of claim 16, wherein the neurodegenerative disease is selected from the group consisting amyotrophic lateral sclerosis, Alzheimer's disease, Parkinson's disease, and polyglutamine diseases.

18-20. (canceled)

21. The cell of claim 13, wherein the cell is a neuron, liver, endothelial, or epithelial cell.

22. A method for identifying a compound useful for treatment of brain pathology, the method comprising:
administering a candidate compound to a genetically engineered non-human animal of claim 12; and
evaluating brain pathology developed by the genetically engineered non-human animal;
wherein reduced brain pathology in the genetically engineered non-human animal indicates the candidate compound is a compound useful for the treatment of a brain pathology.

23. (canceled)

24. The method of claim 22 of 23, wherein brain pathology of the genetically engineered non-human animal to which the compound is administered is compared to the brain pathology of a second genetically engineered non-human animal to which no compound has been administered;
and wherein reduced brain pathology in the treated genetically engineered non-human animal compared to brain pathology in the non-treated genetically engineered non-human animal indicates the candidate compound is a compound useful for the treatment of a brain pathology.

25. The method of claim 22 wherein the brain pathology is neurodegenerative disease, cognitive disorder, or traumatic brain injury.

26. The method of claim 25 wherein the neurodegenerative disease is selected from the group consisting amyotrophic lateral sclerosis, Alzheimer's disease, Parkinson's disease, and polyglutamine diseases.

27. The method of claim 22 wherein brain pathology is evaluated by aggregation of Aβ, p-tau, or a combination thereof, by phosphorylation of tau, by Hsp70 or Hsp110 expression and/or behaviorally.

28-31. (canceled)

32. The method of claim 24 wherein the first animal and second animals are littermates.

33. A method for identifying a compound useful for treatment of brain pathology, the method comprising:
contacting a cell of claim 13 with a candidate compound; and
evaluating brain pathology developed by the cell;
wherein reduced brain pathology in the cell indicates the candidate compound is a compound useful for the treatment of a brain pathology.

34. (canceled)

35. The method of claim 33 wherein the brain pathology of the treated cell is compared to the brain pathology of a cell to which has not been contacted with the candidate compound; and wherein reduced brain pathology in the treated cell compared to brain pathology in the untreated cell indicates the candidate compound is a compound useful for the treatment of a brain pathology.

36. The method of claim 33 wherein the brain pathology is neurodegenerative disease, cognitive disorder, or traumatic brain injury.

37. The method of claim 36 wherein the neurodegenerative disease is selected from the group consisting amyotrophic lateral sclerosis, Alzheimer's disease, Parkinson's disease, and polyglutamine diseases.

38. The method of claim 33 wherein brain pathology is evaluated by aggregation of Aβ, p-tau, or a combination thereof, by phosphorylation of tau, and/or by Hsp70 or Hsp110 expression.

39-42. (canceled)

43. A method of evaluating brain pathology in a biological sample from a subject, the method comprising:
detecting the co-localization of Hsp70 and Aβ and/or the co-localization of Hsp110 and Aβ;
wherein the co-localization of Hsp70 and Aβ and/or the co-localization of Hsp110 and Aβ is indicative of brain pathology.

44. (canceled)

45. A method of treating brain pathology in a subject, the method comprising administering to the subject a compound that increases the expression and/or function of Hsp70 or Hsp110.

46. The method of claim 45 wherein the compound reduces the aggregation of Aβ and/or p-tau, inhibits phosphorylation of tau, reduces or eliminates p-tau, induces dephosphorylation of p-tau, and/or induces degradation of p-tau.

47. A method of detecting a neurodegenerative disease in a subject, the method comprising detecting a polymorphic variant or a mutation in one or more hsp110 alleles in a nucleotide sample obtained from the patient.

48. The method of claim 47 wherein the neurodegenerative disease is selected from the group consisting amyotrophic lateral sclerosis, Alzheimer's disease, Parkinson's disease, and polyglutamine diseases.

49. The method of claim 47 wherein the neurodegenerative disease is presymptomatic.

50. A method of identifying a compound for altering the expression and/or function of heat shock protein 70 (hsp70), the method comprising:
administering a candidate compound to a non-human animal or an isolated cell; and
evaluating the expression and/or function of hsp70 in the animal or cell;
wherein altered expression and/or function of hsp70 in the animal or cell following administration of the compound indicates that the compound is effective for altering the expression and/or function of heat shock protein 70 (hsp70).

51. The method of claim 50, wherein altering the expression and/or function of heat shock protein 70 (hsp70) is reducing the expression and/or function of heat shock protein 70 (hsp70).

52. The method of claim 50, wherein altering the expression and/or function of heat shock protein 70 (hsp70) is increasing the expression and/or function of heat shock protein 70 (hsp70).

53. A method for identifying a compound with anti-angiogenic and/or anti-tumorgenic effect, the method comprising identifying a compound that reduces or eliminates the expression or function of the heat shock protein 70 (Hsp70) in a cell or animal.

54. A method for evaluating carcinogenicity of a candidate compound, the method comprising:
administering the candidate compound to an animal of claim 12;
evaluating a tumor burden attained by the animal; and
comparing the tumor burden attained by the animal to a tumor burden attained by a control animal, wherein a higher tumor burden present in the genetically engineered animal compared to the control animal indicates that the candidate compound is a carcinogen.

55. A method for evaluating carcinogenicity of a candidate compound, the method comprising:
administering the candidate compound to a cell of claim 13;
evaluating the cell for transformation; and
comparing the transformation of the cell to transformation of a control cell, wherein transformation in the cell compared to the control cell indicates the compound is a carcinogen.

56. A method for identifying a candidate anti-carcinogenic compound, the method comprising:
administering a carcinogenic compound to an animal of claim 12;
further administering the candidate anti-carcinogenic compound to the animal;
evaluating a tumor burden attained by the animal; and
comparing the tumor burden attained by the treated animal to a tumor burden attained by a control animal, wherein a lower tumor burden present in the treated animal compared to the control animal indicates the compound is an anti-carcinogenic compound.

57. A method for identifying a candidate anti-carcinogenic compound, the method comprising:
administering a carcinogen to a cell of claim 13;
administering the candidate anti-carcinogenic compound to the cell;
evaluating the cell for transformation; and
comparing the transformation of the cell to transformation of a control cell, wherein transformation in the cell compared to the control cell indicates the compound is a carcinogen.

58-60. (canceled)

61. A method of treating cancer in a subject, the method comprising administering to the subject a compound that reduces the expression and/or function of Hsp70.
